SEQUENCE LISTING (12 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Homo sapiens [sequence omitted]

SEQ ID NO: 3 - DNA, 1535 bp, Homo sapiens
  source 1..1535: mol_type = genomic DNA
agattctccccagacgccgaggatggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctactacaaccagagcgaggccggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccatgaggcggagcagttgagagcctacctggatggcacgtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtccctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaattagaaccttagtataaatttactttctcaaattcttgccatgagaggttgatgagttaattaaaggagaagattcctaaaatttgagagacaaaataaatggaagacatgagaa

SEQ ID NO: 4 - DNA, 1611 bp, Homo sapiens
  source 1..1611: mol_type = genomic DNA
gagaagccaatcagtgtcgtcgcggtcgctgttctaaagtccgcacgcacccaccgggactcagattctccccagacgccgaggatggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgcgggcgccgtggatagagcaggaggggccggagtattgggaccaggagacacggaatatgaaggcccactcacagactgaccgagcgaacctggggaccctgcgcggctactacaaccagagcgaggacggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagagcaccccacccccatgtccaccatgaccctcttcccacgctgacctgtgctccctctccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaattaatggaacgcatgagaaccttccagagtcca

SEQ ID NO: 5 - DNA, 1536 bp, Homo sapiens
  source 1..1536: mol_type = genomic DNA
agagtctcctcagacgccgagatgctggtcatggcgccccgaaccgtcctcctgctgctctcggcggccctggccctgaccgagacctgggccggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtgcagcgacagtgcccagggctctgatgtgtctctcacagcttgaaaagcctgagacagctgtcttgtgagggactgagatgcaggatttcttcacgcctcccctttgtgacttcaagagcctctggcatctctttctgcaaaggcacctgaatgtgtctgcgtccctgttagcataatgtgaggaggtggagagacagcccacccttgtgtccactgtgacccctgttcccatgctgacctgtgtttcctccccagtcatctttcttgttccagagaggtggggctggatgtctccatctctgtctcaactttacgtgcactgagctgcaacttcttacttccctactgaaaataagaatctgaatataaatttgttttctcaaatatttgctatgagaggttgatggattaattaaataagtcaattcctggaatttgagagagcaaataaagacctgagaaccttcca

SEQ ID NO: 6 - DNA, 1542 bp, Homo sapiens
  source 1..1542: mol_type = genomic DNA
acattctccccagaggccgagatgcgggtcatggcgccccgagccctcctcctgctgctctcgggaggcctggccctgaccgagacctgggcctgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacaggctgaccgagtgagcctgcggaacctgcgcggctactacaaccagagcgaggacgggtctcacaccctccagaggatgtctggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctgggagccatcttcccagcccaccatccccatcatgggcatcgttgctggcctggctgtcctggttgtcctagctgtccttggagctgtggtcaccgctatgatgtgtaggaggaagagctcaggtggaaaaggagggagctgctctcaggctgcgtgcagcaacagtgcccagggctctgatgagtctctcatcacttgtaaagcctgagacagctgcctgtgtgggactgagatgcaggatttcttcacacctctcctttgtgacttcaagagcctctggcatctctttctgcaaaggcacctgaatgtgtctgcgttcctgttagcataatgtgaggaggtggagagacagcccacccccgtgtccaccgtgacccctgtccccacactgacctgtgttccctccccgatcatctttcctgttccagagaggtggggctggatgtctccatctctgtctcaaattcatggtgcactgagctgcaacttcttacttccctaatgaagttaagaacctgaatataaatttgtgttctcaaatatttgctatgaagcgttgatggattaattaaataagtcaattcctagaagttgagagagcaaataaagacctgagaaccttccagaa

SEQ ID NO: 7 - DNA, 1586 bp, Homo sapiens
  source 1..1586: mol_type = genomic DNA
tccgcagtcccggttctaaagtccccagtcacccacccggactcacattctccccagaggccgagatgcgggtcatggcgccccgagccctcctcctgctgctctcgggaggcctggccctgaccgagacctgggcctgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaactacaagcgccaggcacaggctgaccgagtgagcctgcggaacctgcgcggctactacaaccagagcgaggacgggtctcacaccctccagaggatgtatggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctgggagccatcttcccagcccaccatccccatcatgggcatcgttgctggcctggctgtcctggttgtcctagctgtccttggagctgtggtcaccgctatgatgtgtaggaggaagagctcaggtggaaaaggagggagctgctctcaggctgcgtgcagcaacagtgcccagggctctgatgagtctctcatcacttgtaaagcctgagacagctgcctgtgtgggactgagatgcaggatttcttcacacctctcctttgtgacttcaagagcctctggcatctctttctgcaaaggcgtctgaatgtgtctgcgttcctgttagcataatgtgaggaggtggagagacagcccacccccgtgtccaccgtgacccctgtccccacactgacctgtgttccctccccgatcatctttcctgttccagagaggtggggctggatgtctccatctctgtctcaaattcatggtgcactgagctgcaacttcttacttccctaatgaagttaagaacctgaatataaatttgtgttctcaaatatttgctatgaagcgttgatggattaattaaataagtcaattcctagaagttgagagagcaaataaagacctgagaaccttccagaa

SEQ ID NO: 8 - DNA, 943 bp, Homo sapiens
  source 1..943: mol_type = genomic DNA
attcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttga

SEQ ID NO: 9 - protein, 6 aa, synthetic construct
HHHHHH

SEQ ID NO: 10 - protein, 51 aa, synthetic construct
  VARIANT 2..51: note = This region may encompass 1-10 GGGGS repeating units
SGGGGSGGGGSGGGGSGGGGSGGGGSGGGGSGGGGSGGGGSGGGGSGGGGS

SEQ ID NO: 11 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 12 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

(19) United States
(12) Patent Application Publication
(10) Pub. No.: US 2025/0161411 A1
(43) Pub. Date: May 22, 2025

(54) TUMOR MHC CLASS I EXPRESSION IS ASSOCIATED WITH INTERLEUKIN-2 RESPONSE IN MELANOMA

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Ingo Mellinghoff, New York, NY (US); Travis J. Hollmann, New York, NY (US); Nicholas Socci, New York, NY (US); Maryam Pourmaleki, New York, NY (US)

(21) Appl. No.: 18/724,546

(22) PCT Filed: Dec. 21, 2022
(86) PCT No.: PCT/US22/53735

§ 371 Date: Jun. 26, 2024

Related U.S. Application Data
(60) Provisional application No. 63/293,986, filed on Dec. 27, 2021.

(51) Int. Cl. A61K 38/20; A61P 35/04; C12Q 1/6886; G01N 33/68

(52) U.S. Cl. CPC A61K 38/2013; A61P 35/04; C12Q 1/6886; G01N 33/6893; G01N 2333/70539; G01N 2800/52

(57) ABSTRACT

The present disclosure provides methods for predicting responsiveness to IL-2 therapy in patients diagnosed with or suffering from melanoma. These methods are based on screening for expression of membranous MHC class I in tumor cells of untreated lesions.

### CROSS-REFERENCE TO RELATED APPLICATIONS

This application is a 371 U.S. National Phase Application of International Application No. PCT/US2022/053735, filed Dec. 21, 2022, which claims the benefit of and priority to U.S. Provisional Patent Application No. 63/293,986, filed Dec. 27, 2021, the contents of each of which are incorporated herein by reference in its entirety.

### GOVERNMENT SUPPORT

This invention was made with government support under CA008748 awarded by the National Institutes of Health. The government has certain rights in the invention.

### TECHNICAL FIELD

The present disclosure relates to methods for predicting responsiveness to IL-2 therapy in patients diagnosed with or suffering from melanoma.

### SEQUENCE LISTING

The instant application contains a Sequence Listing which has been submitted electronically in .xml format and is hereby incorporated by reference in its entirety. Said xml copy, created Jun. 25, 2024, is named 115872-2658_SL and is 24,576 bytes in size.

### BACKGROUND

The following description of the background of the present technology is provided simply as an aid in understanding the present technology and is not admitted to describe or constitute prior art to the present technology.

Up to 20% of patients with metastatic cutaneous melanoma present with “in-transit” metastasis, a form of locoregional recurrence (1). Therapeutic options for in-transit melanoma include surgical excision, isolated limb perfusion, radiation, systemic therapies, and intralesional therapies, including interleukin-2 (IL-2), which was the first FDA-approved immunotherapeutic agent (2, 3). Today, IL-2 is regaining popularity as an immuno-oncology agent as a number of pharmaceutical companies have IL-2 candidates in clinical trials for a variety of solid cancers (4). Compared to systemic IL-2 administration, intralesional IL-2 injection reduces systemic toxicity while maximizing intratumoral IL-2 concentration. Prior studies have reported that 41-96% of injected lesions show a complete response to IL-2 (3, 5). Although responding lesions have been shown to harbor increased densities of CD8⁺ T cells (6, 7), more detailed molecular and cellular effects of intralesional IL-2 and biomarkers of response are unknown.

Accordingly, there is an urgent need for accurate and sensitive biomarkers for predicting responsiveness to intralesional or systemic IL2 therapy in melanoma patients.

SUMMARY OF THE PRESENT TECHNOLOGY

In one aspect, the present disclosure provides a method for selecting a melanoma cancer patient for treatment with IL-2 therapy comprising (a) detecting the presence of wild-type polynucleotides in at least one MHC class I gene in a biological sample obtained from the melanoma cancer patient; and (b) administering to the melanoma cancer patient an effective amount of a therapeutic IL-2 composition. The at least one MHC class I gene may be selected from the group consisting of HLA-A, HLA-B, HLA-C, and B2M. In some embodiments, the wild-type polynucleotides are detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). Additionally or alternatively, in some embodiments, the biological sample comprises genomic DNA, cDNA, RNA, and/or mRNA.

In one aspect, the present disclosure provides a method for selecting a melanoma cancer patient for treatment with IL-2 therapy comprising (a) detecting mRNA and/or polypeptide expression levels of at least one MHC class I gene in a biological sample obtained from the melanoma cancer patient that are comparable to a control sample obtained from a healthy subject or a predetermined threshold; and (b) administering to the melanoma cancer patient an effective amount of a therapeutic IL-2 composition. The at least one MHC class I gene may be selected from the group consisting of HLA-A, HLA-B, HLA-C, and B2M. In another aspect, the present disclosure provides a method for treating melanoma in a patient in need thereof comprising administering to the patient an effective amount of a therapeutic IL-2 composition, wherein mRNA and/or polypeptide expression levels of one or more of HLA-A, HLA-B, HLA-C, and B2M in a biological sample obtained from the patient are comparable to a control sample obtained from a healthy subject or a predetermined threshold. Additionally or alternatively, in some embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). Additionally or alternatively, in certain embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

In any of the preceding embodiments of the methods disclosed herein, the biological sample comprises skin tissue, soft tissue, lymph nodes, whole blood, plasma, or serum. The biological sample may comprise tumor cells and/or macrophages. In certain embodiments, the tumor cells exhibit membranous expression of MHC class I genes.

Additionally or alternatively, in some embodiments of the methods disclosed herein, the therapeutic IL-2 composition comprises wild-type human IL-2, a variant human IL-2 or a chimeric IL-2 fusion polypeptide. In certain embodiments, the wild-type human IL-2 or the chimeric IL-2 fusion polypeptide comprises the amino acid sequence of SEQ ID NO: 1 or SEQ ID NO: 2. Additionally or alternatively, in some embodiments, the chimeric IL-2 fusion polypeptide comprises a Fc domain, a serum albumin domain, a polyethylene glycol (PEG) domain, transferrin, a serum albumin binding protein, a serum immunoglobulin binding protein and a fibronectin (Fn)-based scaffold domain. In any and all embodiments of the methods disclosed herein, the therapeutic IL-2 composition is administered systemically, intratumorally or intravenously.

In any and all embodiments of the methods disclosed herein, the effective amount of the therapeutic IL-2 composition is administered as a series of injections. In certain embodiments, the effective amount of the therapeutic IL-2 composition is administered as a series of 2, 3, 4, 5, 6, 7, 8, 9, 10, 11, 12 or more injections. Additionally or alternatively, in some embodiments, the series of injections are administered over an interval of at least 7 days, at least 14 days, at least 21 days, at least 28 days, at least 35 days, at least 42 days, at least 49 days, at least 56 days, at least 63 days, at least 70 days, at least 80 days, at least 90 days, at least 100 days, at least 110 days, at least 120 days, at least 130 days, at least 140 days, at least 150 days, at least 160 days, at least 170 days, at least 180 days, at least 190 days, at least 200 days, at least 210 days, at least 220 days, at least 230 days, at least 240 days, at least 250 days, at least 260 days, at least 270 days, at least 280 days, or at least 290 days.

In any of the foregoing embodiments of the methods disclosed herein, the patient exhibits stage I, stage II, stage III or stage IV melanoma. The melanoma may be superficial spreading, nodular, lentigo maligna, acral lentiginous, amelanotic, or desmoplastic. In some embodiments, the melanoma is metastatic or localized. Additionally or alternatively, in some embodiments, the patient has a high or low tumor mutation burden (TMB).

### BRIEF DESCRIPTION OF THE DRAWINGS

FIGS. 1A-1C. Multi-dimensional assessment of in-transit melanoma metastases treated with intralesional IL-2. FIG. 1A: Study design. Multiple cutaneous in-transit metastases were excised from each melanoma patient, including at least one untreated (UT) and one IL2-injected lesion. Treatment response for IL2-injected lesions was classified as complete response (CR) or non-CR. FIG. 1B: Allocation of consecutive tumor tissue sections for molecular analyses. FIG. 1C: Summary of molecular analyses completed for each lesion. “Non-responder” refers to all non-/mixed responder patients.

FIGS. 2A-2B. Immune-cell states and gene-expression signatures following tumor-cell eradication by IL-2. FIG. 2A: Shown are cell fractions (rows) with significant changes following IL-2 injection. The forest plot shows the overall effect size (odds ratio) and 95% confidence interval (CI) of each cell fraction across all patients for complete response (CR) (n=101 fields of view, FOVs) versus untreated (n=112 FOVs) and non-CR (n=120 FOVs) versus untreated. The overall median fraction, scaled to 1 for the largest fraction, is shown for untreated, non-CR, and CR lesions. Significant results, determined using a two-sided Wilcoxon test adjusted by Bonferroni correction, are indicated with an asterisk above the median fraction with p-adjusted noted (n.s., not significant). See FIG. 22 for full cell type names. FIG. 2B: The heatmap indicates scaled RNA expression values for differentially expressed genes (p-adjusted<0.0001) in CR (n=6) versus untreated (n=9) and/or non-CR (n=5) versus untreated, sorted by CR versus untreated fold change.

FIGS. 3A-3F. Pre-treatment tumor MHC-I expression is associated with complete tumor response to IL-2. FIG. 3A: Grouping of untreated (UT) lesions from extreme responder and non-/mixed responder patients (labeled “non-responder” throughout figures). FIG. 3B: Shown are cell fractions (rows) with significant differences in untreated lesions from extreme responders (n=38 fields of view, FOVs) versus non-/mixed responders (n=74 FOVs). The left panel shows fractions in each FOV, with overall median, minimum, and maximum (each point represents an FOV). The forest plot shows effect size (odds ratio) and 95% confidence interval (CI) of each cell fraction with p-adjusted noted (two-sided Wilcoxon test adjusted by Bonferroni correction). See FIG. 22 for full cell type names. FIG. 3C: t-SNE of untreated lesion tumor cells colored by patient response and normalized intensity of MHC-I and B2M. FIG. 3D: MHC-I IHC of untreated lesions (scale bar, 50-microns). FIG. 3E: Bar graph showing the percent of untreated lesions with expression of membranous MHC-I in greater than 75% of tumor cells in the initial cohort (extreme responder (n=4) and non-responder (n=5)) and FIG. 3F: in the validation cohort (CR, complete responder (n=6) and non-CR, non-complete responder (n=13)) based on IHC staining (Fisher's exact test, two-sided, exact p-value noted).

FIGS. 4A-4J. Activated tumor microenvironment prior to IL-2 treatment characterizes extreme responders. FIG. 4A: The heatmap indicates scaled RNA expression values for differentially expressed genes (p-adjusted<0.05) in untreated lesions from extreme responders (n=4) versus non-/mixed responders (labeled “non-responder” throughout figures) (n=5), sorted by fold change. FIG. 4B: Schematic of tumor interface analysis. mIF, multiplexed IF. FIG. 4C: Box plots showing B-cell density in tumor and stroma of untreated lesions (minimum, median, and maximum with each point representing a field of view, FOV). Significant results, determined using a two-sided Wilcoxon test adjusted by Bonferroni correction, are indicated with an asterisk (p-adjusted<0.05). FIG. 4D: Total B-cell aggregate counts in untreated lesions. FIG. 4E: Total count of B cells per aggregate (Wilcoxon rank sum test, exact p-value noted) in untreated lesions (extreme responder (n=10) and non-responder (n=4)). FIG. 4F: Representative multiplexed IF images from an untreated lesion of an extreme responder (6_4) and non-/mixed responder (1_1) showing B cell aggregates. FIG. 4G: Cartoon of CD8⁺ T-cell states. FIG. 4H: Mean density of CD8⁺ T cells expressing all combinations of PD-1/TIM-3/LAG-3 in untreated lesions in 10-micron intervals from-360:360-microns. FIG. 4I: Box plots showing density of CD8⁺ T cells expressing all combinations of PD-1/TIM-3/LAG-3 in tumor and stroma of untreated lesions (minimum, median, and maximum with each point representing an FOV). FIG. 4J: CD8⁺ T-cell neighborhood definitions. The heatmap indicates effect size (odds ratio) of each cell fraction (rows) in untreated lesions for SP/DP/TP neighborhoods normalized against TN neighborhoods. See also FIG. 29.

FIGS. 5A-5E. Selection of antibody panel and validation of multiplexed immunofluorescence staining. FIG. 5A: Antibody panel. MHC-II was used as both a cell identity and cell function marker. See also FIG. 21. FIG. 5B: Example of validation process shown for PD-L1 on normal human placenta. Representative images showing 3 dilutions of PDLI Cy5 direct conjugate, PD-L1 primary with an anti-rabbit IgG Alexa Fluor 647 secondary, PDL 1 immunohistochemistry (IHC), and hematoxylin and eosin (H&E) stained on adjacent tissue sections. FIG. 5C: Representative images showing the optimal staining concentration for each direct conjugate on appropriate normal human controls. Skin: B2M, CD68, CD163, MRC1, PCK26 (panCK), SOX-10. Lymph node: CD3, CD4, CD8, CD20, CD27, CD45, KI-67. Tonsil: B7-H3, CD14, CD25, FOXP3, ICOS, IDO-1 LAG-3, MHC-I, MHC-II, PD-1, TGM2, TIM-3. Colon: CD56, S100B. Placenta: PD-L1. FIG. 5D: Representative images showing the validation process for epitope stability to dye inactivation for CD27, PD-1, KI-67, and MHC-I on normal human lymph node. FIG. 5E: Representative images showing images acquired using multiplexed immunofluorescence (Cell Dive) and IHC for lesion 3_2. All scale bars are 100-microns.

FIGS. 6A-6C. Genomic profile of in-transit melanoma metastases. FIG. 6A: Genomewide view of gene copy number alterations for the indicated untreated (UT) and non-complete response (non-CR) lesions ordered by lesion ID. Regions of copy gains and loss are indicated in red and blue, respectively. Numbers refer to chromosomal location. FIG. 6B: Genetic alterations in the untreated and non-CR lesions in the cohort ordered by temporality. FIG. 6C: Genetic alterations in the untreated and non-CR lesions in the MSK-IMPACT cutaneous melanoma cohort. Fraction of lesions altered for each gene in the MSK-IMPACT cohort is noted. N/P, not profiled.

FIGS. 7A-7G. Phenotypic data for T cells, B cells, NK cells, and myeloid cells. FIG. 7A: Single-cell proteomic analysis approach. Derivation of 664 distinct cell states quantified as 685 distinct cell fractions using combinations of cell types and cell function markers. See FIG. 22 for full cell type names. FIG. 7B: Identification of 16 different immune cell types using single-cell proteomics. t-distributed stochastic neighbor embedding (1-SNE) of all immune cells from 22 lesions colored by cell type. FIG. 7C: 1-SNE of immune cells colored by normalized intensity of each cell function marker. FIGS. 7D-7G: 1-SNE of all T cells (FIG. 7D), B cells (FIG. 7E), natural killer cells (FIG. 7F), and macrophage/monocytes (FIG. 7G) from 22 lesions colored by the normalized intensity of each cell function marker, lesion response (non-complete response, non-CR; complete response, CR), patient response, subtype, and distance from the interface. Grey cells indicate cells with missing information (ICOS) and cells that are not within 360-microns of the tumor interface (distance to interface).

FIGS. 8A-8C. Intra-tumoral heterogeneity in immune cell composition. FIG. 8A: Location of all fields of view (FOVs) in all lesions (n=22). Interface FOVs (yellow) cover approximately 50% tumor and 50% stroma, center FOVs (red) cover 100% tumor, and regressed FOVs (green) cover regions of regressed tumor. See also FIG. 24. FIG. 8B: Number of FOVs examined per lesion. FIG. 8C: Intratumoral heterogeneity in immune cell infiltrates. Shown is hierarchical clustering of immune cell fractions over total immune cells in each FOV (n=333). FOVs belonging to the same lesion are indicated in the same color (column annotations). For cell type abbreviations, see FIG. 22.

FIG. 9. Increase in triple negative PD-1/LAG-3/TIM-3 CD8⁺ T cells in lesions that completely regress following intralesional interleukin-2 injection. Representative multiplexed immunofluorescence (IF) and pseudo-colored images for untreated (UT) lesion 1_1 field of view (FOV) 7, non-complete response (non-CR) lesion 1_2 FOV 25, UT lesion 4_1 FOV 4, and CR lesion 4_4 FOV 3. The fraction of triple negative PD-1/LAG-3/TIM-3 CD8⁺ T cells over total CD8⁺ T cells in each FOV are 0.110, 0.636, 0.249, and 0.777, respectively.

FIGS. 10A-10B. Decrease in TIM-3 positive CD8+ T cells in lesions that completely regress following intralesional interleukin-2 injection. FIG. 10A: Representative images showing CD8 (red) and TIM-3 (white) in untreated (UT) lesion 4_2, non-complete response (non-CR) lesion 1_2, and complete response (CR) lesion 4_4 acquired using Cell Dive and a different multiplexed immunofluorescence method (validation assay—see methods). Scale bar is 10-microns. FIG. 10B: Summary of quantified fractions of TIM-3 positive CD8⁺ T cells (Cell Dive) and TIM-3 positive CD8⁺/CD4− cells (validation assay). Cell Dive fractions are computed over sum of all fields of view. Validation assay fractions are computed over the entire tissue (whole slide).

FIGS. 11A-11B. Untreated lesions from the same patient show a similar immune cell composition. FIG. 11A: Box plots (center line: median fraction, box limits: upper and lower quartiles, whiskers: 1.5x interquartile range, points: field of view (FOV) fractions) comparing the fraction of each immune cell type (n=16) over total immune cells using a two-sided Wilcoxon test adjusted by Bonferroni correction for untreated (UT) lesions 4_1 (n=6 FOVs) and 4_2 (n=11 FOVs) from patient 4 and (FIG. 11B) for UT lesions 6_1 (n=8 FOVs) and 6_4 (n=13 FOVs) from patient 6. n.s., not significant (p-adjusted>0.001). For exact p-values, see FIG. 25. For cell type abbreviations, see FIG. 22.

FIG. 12. Intralesional interleukin-2 associated changes in immune cell state fractions. Shown are cell fractions (rows) with significant changes following interleukin-2 (IL-2) injection. The forest plot shows the overall effect size and 95% confidence interval (CI) of each cell fraction across all patients for complete response (CR) (n=101 fields of view, FOVs) versus untreated (n=112 FOVs) and non-complete response (non-CR) (n=120 FOVs) versus untreated. The overall median fraction, scaled to 1 for the largest fraction, is shown for untreated, non-CR, and CR lesions. Significant results, determined using a two-sided Wilcoxon test adjusted by Bonferroni correction, are indicated with an asterisk above the median fraction with p-adjusted noted (n.s., not significant). The heatmap indicates the effect size of each cell fraction in the IL-2 injected lesion from a single patient versus the untreated lesion(s) from the same patient. Red denotes an increase in cell fraction, blue denotes a decrease. The asterisks in the heatmap indicate significance when effect size is in the same direction as the overall. The FOV counts for intra-patient CR/UT are 21/12, 13/9, 16/17, 19/17, 15/17, 10/21, 7/21 and for intrapatient non-CR/UT are 22/12, 23/9, 17/14, 21/14, 33/35, 4/4 (in order listed above). See FIG. 22 for full cell type names.

FIG. 13. Intralesional interleukin-2 associated changes in immune cell state densities. Shown are cell densities (rows) with significant changes following interleukin-2 (IL-2) injection. The forest plot shows the overall effect size and 95% confidence interval (CI) of each cell density across all patients for complete response (CR) (n=101 fields of view, FOVs) versus untreated (UT) (n=112 FOVs) and non-complete response (non-CR) (n=120 FOVs) versus untreated. The overall median density, scaled to 1 for the largest density, is shown for untreated, non-CR, and CR lesions. Significant results, determined using a two-sided Wilcoxon test adjusted by Bonferroni correction, are indicated with an asterisk above the median density with p-adjusted noted (n.s., not significant). The heatmap indicates the effect size of each cell density in the IL-2 injected lesion from a single patient versus the untreated lesion(s) from the same patient. Red denotes an increase in cell density, blue denotes a decrease. The asterisks in the heatmap indicate significance when effect size is in the same direction as the overall. The FOV counts for intra-patient CR/UT are 21/12, 13/9, 16/17, 19/17, 15/17, 10/21, 7/21 and for intrapatient non-CR/UT are 22/12, 23/9, 17/14, 21/14, 33/35, 4/4 (in order listed above). For cell type abbreviations, see FIG. 22.

FIG. 14. Determinants of response to intralesional interleukin-2 (cell densities). Shown are cell densities (rows) with significant differences in untreated lesions from extreme responders (n=38 fields of view, FOVs) versus non-/mixed responders (labeled “nonresponder” in figure) (n=74 FOVs). The left panel shows densities in each FOV, with overall median, minimum, and maximum. The forest plot shows effect size and 95% confidence interval (CI) of each cell density with p-adjusted, determined using a two-sided Wilcoxon test adjusted by Bonferroni correction, noted. For cell type abbreviations, see FIG. 22.

FIG. 15. Expression of B2M/MHC-I/MHC-II in macrophage/monocytes in untreated lesions is associated with IL-2 response. Violin plot showing fraction of triple positive B2M/MHC-I/MHC-II macrophage/monocytes in untreated lesions from extreme responders (n=38 fields of view, FOVs) versus non-/mixed responders (labeled “non-responder” in figure) (n=74 FOVs) (with overall median, minimum, and maximum shown). The forest plot shows effect size and 95% confidence interval (CI) with p-adjusted noted. See FIG. 22 for full cell type names.

FIGS. 16A-16G. Increased RNA expression of B2M and HLA genes in untreated lesions of extreme responders. FIG. 16A: Oncoplot showing genetic alterations in antigen presentation pathway genes. “Non-responder” refers to all non-/mixed responder patients. FIG. 16B: Heatmap showing MHC-I scores and scaled RNA expression values of B2M, HLA-A, HLA-B, and HLA-(in untreated lesions from extreme responders and non-/mixed responders. FIGS. 16C-16F: Boxplots showing expression of (FIG. 16C) B2M, (FIG. 16D) HLA-A, (FIG. 16E) HLA-B, and (FIG. 16F) HLA-C genes in untreated lesions of extreme responders (n=4) and non-/mixed responders (n=5). p-values are derived from a two-sided Wilcoxon test. FIG. 16G: Correlation (Pearson) of untreated lesion tumor cell MHC-I membrane positivity (%) with B2M RNA expression value.

FIG. 17. Activated tumor microenvironment in tumor MHC-I high neighborhoods. Analysis approach to defining tumor MHC-I high neighborhoods (minimum 75% MHC-I positive tumor cells) and tumor MHC-I low neighborhoods (maximum 25% MHC-I positive tumor cells). Shown are cell fractions (rows) with significant differences in tumor MHCI high versus tumor MHC-I low neighborhoods from untreated lesions.

FIG. 18. Untreated lesions of extreme responders and non-/mixed responders exhibit similar expression of cell identity genes. Volcano plot showing differential expression of genes (n=770) in untreated lesions of extreme responders (n=4) versus non-/mixed responders (labeled “non-responder” in figure) (n=5). Cell identity genes (false discovery rate, FDR30.05) are labeled in black.

FIGS. 19A-19B. Fraction of triple positive PD-1/LAG-3/TIM-3 CD8⁺ T cells increases as CD8⁺ T cells approach the tumor-stroma interface. FIG. 19A: Fractional distribution of CD8⁺ T cells expressing all combinations of PD-1/TIM-3/LAG-3 in untreated lesions of extreme responders (n-31 fields of view, FOVs) and non-/mixed responders (labeled “non-responder” in figure) (n=54 FOVs) in 10-micron intervals from-360:360-microns. TN, triple negative. SP, single positive. DP, double positive. TP, triple positive. FIG. 19B: Fraction of CD8⁺ T cells triple positive for PD-1/TIM-3/LAG-3 in untreated lesions of extreme responders and non-/mixed responders in 10-micron intervals from-360:0 (tumor) and 0:360 (stroma). Kendall's tau (t) and p-value, determined using a two-sided Mann-Kendall test, and Sen's slope are noted for each side of the tumor interface (tumor and stroma).

FIG. 20. Patient characteristics. Clinical information for 7 patients and 27 in-transit melanoma metastases. Abbreviations: No., number; Non-Responder, Non-/Mixed Responder; IL-2, interleukin-2; NA, not applicable; UT, untreated; non-CR, non-complete response; CR, complete response.

FIG. 21. Antibody information. Abbreviations: IF, immunofluorescence; IHC, immunohistochemistry.

FIG. 22. Use of cell identity markers. Each cell type is defined by a combination of positive and negative staining results for specific cell identity markers.

FIG. 23. List of cell states, cell fractions, and cell densities. First tab “Cell_states” includes the list of 664 cell states. Second tab “Cell_fractions” includes the list of 685 cell fractions with cell state (numerator) and population (denominator) noted. Third tab “Cell_densities” includes the list of 664 cell densities. Fourth tab “Summary” includes a summary of cell states. Abbreviations: No, number. For cell type abbreviations, see FIG. 22.

FIG. 24. Summary of cell loss and fields of view for multiplexed immunofluorescence data. Abbreviations: No., number; FOVs, fields of view.

FIG. 25. Comparison of multiple untreated lesions from the same patient. Statistics for immune cell fractions from patient 4 are included in the first tab “patient_4” and from patient 6 are included in the second tab “patient_6”. For cell type abbreviations, see FIG. 22. Abbreviations: CI, 95% confidence interval.

FIG. 26. Validation cohort patient characteristics. Clinical information for metastatic melanoma patients in the validation cohort (n=19) treated with either intralesional interleukin-2 (IL-2) or high-dose systemic IL-2. Patient response was classified as complete responder (CR) or non-complete responder (non-CR). Abbreviations: Calgary, University of Calgary; NIH, National Institutes of Health; MDACC, MD Anderson Cancer Center; JHMI, Johns Hopkins Medical Institute.

FIG. 27. Summary of MHC-I scores in initial and validation cohorts. First tab “MHCI_scores” includes the score (fraction of tumor cells with membranous MHC-I positivity) for all patients. Second tab “MHCI_scores_summary” includes a summary of MHC-I scores grouped by over 75% tumor cell positive and under 75% tumor cells positive for the initial and validation cohorts. Abbreviations: MSKCC, Memorial Sloan Kettering Cancer Center; Calgary, University of Calgary; NIH, National Institutes of Health; MDACC, MD Anderson Cancer Center; JHMI, Johns Hopkins Medical Institute; CR, complete responder; non-CR, noncomplete responder.

FIG. 28. High tumor mutation burden in untreated lesions is not associated with response to intralesional IL-2. Tumor mutation burden is measured in mutations per megabase. Abbreviations: Non-responder, Non-/mixed responder; UT, untreated.

FIG. 29. Cellular neighborhoods of CD8+ T cells with an exhausted phenotype. Includes raw data for FIG. 4J. For cell type abbreviations, see FIG. 22. Abbreviations: LO, log odds; LOR, log odds ratio.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Clinical response to ICB has been extensively studied and associated with both tumor cell-intrinsic factors (including TMB, HLA expression, and PD-L1 expression) (17, 21) and tumor microenvironment factors (including TLS and dysfunctional T cells) (16, 18, 22). There are presently no known biomarkers of response for IL-2.

Cancer immunotherapy can result in lasting tumor regression, but predictive biomarkers of treatment response remain ill-defined. Studies have shown that not all “hot” tumors are responsive to immunotherapy. Havel et al., Nat Rev Cancer. 2019 March; 19 (3): 133-150; Rodig et al., Sci. Transl. Med. 10, eaar3342 (2018). Indeed, identifying biomarkers for immunotherapy is both complex and unpredictable. For example, expression of MHC-I on the tumor-cell membrane in melanoma has also been reported to be associated with clinical response to anti-CTLA-4, but not anti-PD-1 ICB (23). Similarly, it has been discovered that neither TMB nor high T cell densities are reliable predictors of responsiveness to immunotherapy. Lu et al., JAMA Oncol. 2019; 5 (8): 1195-1204; Chowell et al., Nature Biotechnology 40:499-506 (2022).

As disclosed herein, single-cell proteomics, transcriptomics, and genomics were performed on matched untreated and interleukin-2 (IL-2) injected metastases from patients with melanoma. Lesions that completely regressed following intralesional IL-2 harbored increased fractions and densities of non-proliferating CD8⁺ T cells lacking expression of PD-1, LAG-3 and TIM-3 (PD-1⁻LAG-3⁻TIM-3⁻). Untreated lesions from patients who subsequently responded with complete eradication of all tumor cells in all injected lesions (individuals referred to herein as “extreme responders”) were characterized by proliferating CD8⁺ T cells with an exhausted phenotype (PD-1⁺LAG-3⁺TIM-3⁺), stromal B-cell aggregates, and expression of IFNγ and IL-2 response genes. Loss of membranous MHC class I expression in tumor cells of untreated lesions was associated with resistance to IL-2 therapy. This finding was validated in an independent cohort of metastatic melanoma patients treated with intralesional or systemic IL-2.

The present disclosure uses single-cell proteomics and bulk transcriptomics and genomics to identify changes in the tumor microenvironment and biomarkers of response associated with intralesional IL-2 therapy for patients with melanoma while describing a multi-dimensional approach for biomarker hypothesis development. These results demonstrate that intact tumor cell antigen presentation is required for melanoma response to IL-2.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

“Antigen presenting cell” as used herein refers to a cell that displays foreign antigen complexed with MHC on its surface.

As used herein, “antigenic peptide” refers to a peptide molecule that is bound or capable of binding to the binding groove of either MHC class I or MHC class II.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. In some embodiments, the epitope is a conformational epitope or a non-conformational epitope.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter-none; strand-both; cutoff-60; expect=10; Matrix=BLOSUM62; Descriptions=50 sequences; sort by =HIGH SCORE; Databases-non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the term “IL-2 polypeptide” or “IL-2” or “IL2” or “IL-2 protein” or “interleukin (IL)-2,” refers to a pleiotropic cytokine that activates and induces proliferation of T cells and natural killer (NK) cells. IL-2 signals by binding its receptor, IL-2R, which is comprised of alpha, beta, and gamma subunits and stimulates proliferation of antigen-activated T cells. “IL-2 polypeptide” encompasses mammal wild type Interleukin-2, and variants thereof. In some embodiments, the IL-2 polypeptide comprises the amino acid sequence of native human IL-2 precursor (SEQ ID NO: 1) or mature human IL-2, which is the IL-2 sequence less the 20 amino acid N-terminal signal peptide (SEQ ID NO: 2)

(SEQ ID NO: 1)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT
(SEQ ID NO: 2)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT

Active variants of IL-2 have been disclosed in the literature. Variants of the native IL-2 can be fragments, analogues, and derivatives thereof. By “fragment” is intended a polypeptide comprising only a part of the polypeptide sequence. An “analogue” designates a polypeptide comprising the native polypeptide sequence with one or more amino acid substitutions, insertions, or deletions. “Derivatives” include any modified native IL-2 polypeptide or fragment or analogue thereof, such as glycosylated, phosphorylated, fused to another polypeptide or molecule, polymerized, etc., or through chemical or enzymatic modification or addition to improve the properties of IL-2 (e.g., stability, specificity, etc.). The IL-2 moiety of active variants generally has at least 75%, preferably at least 80%, 85%, more preferably at least 90% or at least 95% amino acid sequence identity to the amino acid sequence of the reference IL-2 polypeptide, for instance mature wild type human IL-2 (SEQ ID NO: 1).

Examples of IL-2 variants are disclosed, for instance, in EP109748, EP136489, U.S. Pat. No. 4,752,585; EP200280, EP118617, WO99/60128, EP2288372, U.S. Pat. Nos. 9,616,105, 9,580,486, WO2010/085495, WO2016/164937, which are herein incorporated by reference in their entirety. The IL-2 polypeptides used herein may be isolated from a variety of sources, such as from human tissue types or from another source, or prepared by recombinant or synthetic methods. Additionally, the IL-2 for use in the present disclosure may be a product of a recombinant method wherein the IL-2 encoding DNA is administered to a subject, for example, such as cytokine therapy. The IL-2 of the present disclosure may also be modified in a way to form a chimeric molecule comprising IL-2 fused to another heterologous polypeptide or amino acid sequence.

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the terms “individual”, “patient”, or “subject” can be an individual organism, a vertebrate, a mammal, or a human. In some embodiments, the individual, patient or subject is a human.

In general, the term “MHC molecule” denotes the major histocompatibility complex and is intended to include alleles. The MHC molecule can suitably be a vertebrate MHC molecule such as a human, a mouse, a rat, a porcine, a bovine or an avian MHC molecule. Such MHC complexes from different species have different names. E.g. in humans, MHC complexes are denoted HLA. The person skilled in the art will readily know the name of the MHC complexes from various species. By way of example, in humans e.g. HLA A, HLA B, HLA C, HLA D, HLA E, HLA F, HLA G, HLA H, HLA DR, HLA DQ and HLA DP alleles are of interest, and in the mouse system, H-2 alleles are of interest. Likewise, in the rat system RT1-alleles, in the porcine system SLA-alleles, in the bovine system BoLA, in the avian system e.g. chicken-B alleles, are of interest. “MHC protein” and “MHC molecule” are used interchangeably herein. Accordingly, a functional MHC peptide complex comprises a MHC protein or MHC molecule associated with a peptide to be presented for cells or binding partners having an affinity for said peptide.

As used herein, the terms “MHC complexes” as used herein are meant such complexes which are capable of performing at least one of the functions attributed to said complex. The terms include both classical and non-classical MHC complexes. The meaning of “classical” and “non-classical” in connection with MHC complexes is well known to the person skilled in the art. Non-classical MHC complexes are subgroups of MHC-like complexes. The term “MHC complex” includes MHC Class I molecules, MHC Class II molecules, as well as MHC-like molecules (both Class I and Class II), including the subgroup non-classical MHC Class I and Class II molecules. As used herein, “MHC complex” is used interchangeably with MHC-peptide complex, unless it is specified that the MHC complex is empty, i.e. is not complexed with peptide.

A “MHC Class I molecule” as used herein is defined as a molecule which comprises 1-3 subunits, including a heavy chain, a heavy chain combined with a light chain (beta2m), a heavy chain combined with a light chain (beta2m) through a flexible linker, a heavy chain combined with a peptide, a heavy chain combined with a peptide through a flexible linker, a heavy chain/beta2m dimer combined with a peptide, and a heavy chain/beta2m dimer with a peptide through a flexible linker to the heavy or light chain. The MHC molecule chain can be changed by substitution of single or by cohorts of native amino acids or by inserts, or deletions to enhance or impair the functions attributed to said molecule. By example, it has been shown that substitution of XX with YY in position nn of human beta2m enhance the biochemical stability of MHC Class I molecule complexes and thus can lead to more efficient antigen presentation of subdominant peptide epitopes. MHC Class I like molecules (including non-classical MHC Class I molecules) include CD1d, HLA E, HLA G, HLA F, HLA H, MIC A, MIC B, ULBP-1, ULBP-2, and ULBP-3.

A “MHC Class II molecule” as used herein refers to a molecule which comprises 2-3 subunits including an alpha-chain and a beta-chain (alpha/beta-dimer), an alpha/beta dimer with a peptide, and an alpha/beta dimer combined with a peptide through a flexible linker to the alpha or beta chain, an alpha/beta dimer combined through an interaction by affinity tags e.g. jun-fos, an alpha/beta dimer combined through an interaction by affinity tags e.g. jun-fos and further combined with a peptide through a flexible linker to the alpha or beta chain. The MHC molecule chains can be changed by substitution of single or by cohorts of native amino acids or by inserts, or deletions to enhance or impair the functions attributed to said molecule. Under circumstances where the alpha-chain and beta-chain have been fused, to form one subunit, the “MHC Class II molecule” can comprise only 1 subunit. MHC Class II like molecules (including non-classical MHC Class II molecules) include HLA DM, HLA DO, I-A beta2, and I-E beta2.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴M, 10⁻⁵M, 10⁻⁶M, 10⁻⁷M, 10⁻⁸ M, 10⁻⁹M, 10⁻¹⁰M, 10⁻¹¹ M, or 10⁻¹² M. The term “specifically binds” may also refer to binding where a molecule (e.g., an antibody or antigen binding fragment thereof) binds to a particular polypeptide, or an epitope on a particular polypeptide, without substantially binding to any other polypeptide, or polypeptide epitope.

As used herein, the term “simultaneous” therapeutic use refers to the administration of at least two active ingredients by the same route and at the same time or at substantially the same time.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### IL-2 Therapeutic Compositions

Interleukin-2 (IL-2) is a cytokine that induces proliferation of antigen-activated T cells and stimulates natural killer (NK) cells. The biological activity of IL-2 is mediated through a multi-subunit IL-2 receptor complex (IL-2R) of three polypeptide subunits that span the cell membrane: p55 (IL-2Rα, the alpha subunit, also known as CD25 in humans), p75 (IL-2Rβ, the beta subunit, also known as CD 122 in humans) and p64 (IL-2Rγ, the gamma subunit, also known as CD 132 in humans). T cell response to IL-2 depends on a variety of factors, including: (1) the concentration of IL-2; (2) the number of IL-2R molecules on the cell surface; and (3) the number of IL-2R occupied by IL-2 (i.e., the affinity of the binding interaction between IL-2 and IL-2R. The IL-2: IL-2R complex is internalized upon ligand binding and the different components undergo differential sorting. IL-2Rα is recycled to the cell surface, while IL-2 associated with the IL-2: IL-2Rβγ complex is routed to the lysosome and degraded. When administered as an intravenous (i.v.) bolus, IL-2 has a rapid systemic clearance (an initial clearance phase with a half-life of 12.9 minutes followed by a slower clearance phase with a half-life of 85 minutes) (Konrad et al., Cancer Res. 50:2009-2017, 1990). Outcomes of systemic IL-2 administration in cancer patients are far from ideal. While 15 to 20 percent of patients respond objectively to high-dose IL-2, the great majority do not, and many suffer severe, life-threatening side effects, including nausea, confusion, hypotension, and septic shock. Accordingly, accurate and sensitive biomarkers for predicting responsiveness to intralesional or systemic IL-2 therapy are urgently needed.

### IL-2 and Variants Thereof

In certain embodiments, an effective amount of IL-2 or an extended-pharmacokinetic (PK) IL-2 (e.g., a fusion protein containing at least IL-2 and a heterologous polypeptide, such as a hexa-histidine tag (SEQ ID NO:9) or hemagglutinin tag or an Fc region or human serum albumin) is administered systemically, intratumorally or intravenously. In one embodiment, the IL-2 is a human recombinant IL-2 such as Proleukin® (aldesleukin). Proleukin® is a human recombinant interleukin-2 product produced in E. coli. Proleukin® differs from the native interleukin-2 in the following ways: a) it is not glycosylated; b) it has no N-terminal alanine; and c) it has serine substituted for cysteine at amino acid positions 125. Proleukin® exists as biologicially active, non-covalently bound microaggregates with an average size of 27 recombinant interleukin-2 molecules.

In some aspects, the extended-PK IL-2 comprises the amino acid sequence of wild-type IL-2 (e.g., human IL-2 in its precursor form (SEQ ID NO: 1) or mature IL-2 (SEQ ID NO: 2)). In certain embodiments, the extended-PK IL-2 is mutated such that it has a higher affinity for the IL-2R alpha receptor compared with unmodified IL-2. Increasing the affinity of IL-2 for IL-2Rα at the cell surface will increase receptor occupancy within a limited range of IL-2 concentration, as well as raise the local concentration of IL-2 at the cell surface. Exemplary IL-2 mutants which are high affinity binders include those described in WO2013/177187A2 and U.S. Pat. No. 7,569,215, the contents of which are incorporated herein by reference. In certain embodiments, the IL-2 moiety of the extended-PK IL-2 is wild-type human IL-2 or a variant human IL-2.

In some embodiments, the IL-2 polypeptide comprises an amino acid sequence that is at least 70% identical, at least 75% identical, at least 80% identical, at least 85% identical, at least 90% identical, at least 95% identical, at least 97% identical, at least 98% identical, or at least 99% identical to an amino acid sequence of wild-type IL-2 in its precursor form or the mature form (e.g., SEQ ID NO: 1 or SEQ ID NO: 2) that bind IL-2Rα. For example, an IL-2 variant polypeptide can have at least one mutation (e.g., a deletion, addition, or substitution of 1, 2, 3, 4, 5, 6, 7, 8, 9, 10, 11, 12, 13, 14, 15, 16, 17, 18, 19, 20, or more amino acid residues) that increases the affinity for the alpha subunit of the IL-2 receptor relative to wild-type IL-2. By way of illustration, a polypeptide that includes an amino acid sequence that is at least 95% identical to a reference amino acid sequence of SEQ ID NO: 1 or SEQ ID NO: 2 is a polypeptide that includes a sequence that is identical to the reference sequence except for the inclusion of up to five alterations of the reference amino acid of SEQ ID NO: 1 or SEQ ID NO: 2. For example, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino (N—) or carboxy (C—) terminal positions of the reference amino acid sequence or anywhere between those terminal positions, interspersed either individually among residues in the reference sequence or in one or more contiguous groups within the reference sequence. The substituted amino acid residue(s) can be, but are not necessarily, conservative substitutions, which typically include substitutions within the following groups: glycine, alanine; valine, isoleucine, leucine; aspartic acid, glutamic acid; asparagine, glutamine; serine, threonine; lysine, arginine; and phenylalanine, tyrosine. These mutations can be at amino acid residues that contact IL-2Rα.

In general, the polypeptides suitable for use in the methods disclosed herein may be synthetic, or produced by expression of a recombinant nucleic acid molecule. In the event the polypeptide is an extended-PK IL-2 (e.g., a fusion protein containing at least IL-2 and a heterologous polypeptide, such as a hexa-histidine tag (SEQ ID NO: 9) or hemagglutinin tag or an Fc region or human serum albumin), it can be encoded by a hybrid nucleic acid molecule containing one sequence that encodes IL-2 and a second sequence that encodes all or part of the heterologous polypeptide. The techniques that are required to make IL-2 polypeptides are routine in the art, and can be performed without resort to undue experimentation by one of ordinary skill in the art. For example, a mutation that consists of a substitution of one or more of the amino acid residues in IL-2 can be created using a PCR-assisted mutagenesis technique. Mutations that consist of deletions or additions of amino acid residues to an IL-2 polypeptide can also be made with standard recombinant techniques. In the event of a deletion or addition, the nucleic acid molecule encoding IL-2 is simply digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences. In addition to generating IL-2 mutants via expression of nucleic acid molecules that have been altered by recombinant molecular biological techniques, IL-2 mutants can be chemically synthesized. Chemically synthesized polypeptides are routinely generated by those of skill in the art. As noted above, IL-2 can also be prepared as fusion or chimeric polypeptides that include IL-2 and a heterologous polypeptide (i.e., a polypeptide that is not IL-2). The heterologous polypeptide can increase the circulating half-life of the chimeric polypeptide in vivo, and may, therefore, further enhance the properties of IL-2. The polypeptide that increases the circulating half-life may be serum albumin, such as human or mouse serum albumin.

In certain embodiments, the chimeric polypeptide can include IL-2 and a polypeptide that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In certain embodiments, the chimeric polypeptide further comprises a C-terminal c-myc epitope tag. Chimeric polypeptides can be constructed using no more than conventional molecular biological techniques, which are well within the ability of those of ordinary skill in the art to perform.

### Nucleic Acid Molecules Encoding IL-2

IL-2, either alone or as a part of a chimeric polypeptide, can be obtained by expression of a nucleic acid molecule. Thus, nucleic acid molecules encoding polypeptides containing IL-2 or an IL-2 variant are considered suitable for use in the methods disclosed herein. In some embodiments, the nucleic acid molecule encoding an IL-2 variant can be at least 75%, at least 85%, or at least 95% (e.g., 99%) identical to the nucleic acid encoding full length wild-type IL-2 (e.g., SEQ ID NO: 1) or wild-type IL-2 without the signal peptide (e.g., SEQ ID NO: 2).

The nucleic acid molecules suitable for use in the methods disclosed herein contain naturally occurring sequences, or sequences that differ from those that occur naturally, but, due to the degeneracy of the genetic code, encode the same polypeptide. These nucleic acid molecules can comprise RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand). The nucleic acid molecules are not limited to sequences that encode polypeptides; some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The isolated nucleic acid molecules can include fragments not found as such in the natural state. Thus, the present disclosure encompasses use of recombinant molecules, such as those in which a nucleic acid sequence (for example, a sequence encoding an IL-2 variant) is incorporated into a vector (e.g., a plasmid or viral vector) or into the genome of a heterologous cell (or the genome of a homologous cell, at a position other than the natural chromosomal location).

As described above, IL-2 variants suitable for use in the methods disclosed herein may exist as a part of a chimeric polypeptide. In addition to, or in place of, the heterologous polypeptides described above, a nucleic acid molecule suitable for use in the methods disclosed herein can contain sequences encoding a “marker” or “reporter.” Examples of marker or reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-hosphotransferase (HPH), thymidine kinase (TK), lacz (encoding β-galactosidase), and xanthine guanine phosphoribosyl transferase (XGPRT). Skilled artisans will be aware of additional useful reagents, for example, of additional sequences that can serve the function of a marker or reporter. The nucleic acid molecules suitable for use in the methods disclosed herein can be obtained by introducing a mutation into IL-2-encoding DNA obtained from any biological cell, such as the cell of a mammal. Thus, the nucleic acids (and the polypeptides they encode) can be those of a mouse, rat, guinea pig, cow, sheep, horse, pig, rabbit, monkey, baboon, dog, or cat. Typically, the nucleic acid molecules will be those of a human.

### Extended-PK Groups

As described herein, IL-2 or variant IL-2 may be fused to an extended-PK group, which increases circulation half-life. Non-limiting examples of extended-PK groups include, but are not limited to Fc domains, serum albumin domains, polyethylene glycol (PEG) domains, transferrin, serum albumin binding proteins, serum immunoglobulin binding proteins and fibronectin (Fn)-based scaffold domains. Extended-PK groups are described in detail in WO2016025645, the contents of which are incorporated herein by reference in their entirety.

In certain embodiments, the serum half-life of extended-PK IL-2 is increased relative to IL-2 alone (i.e., IL-2 not fused to an extended-PK group). In certain embodiments, the serum half-life of extended-PK IL-2 is at least 20, 40, 60, 80, 100, 120, 150, 180, 200, 400, 600, 800, or 1000% longer relative to the serum half-life of IL-2 alone. In certain embodiments, the serum half-life of the extended-PK IL-2 is at least 1.5-fold, 2-fold, 2.5-fold, 3-fold, 3.5 fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of IL-2 alone. In certain embodiments, the serum half-life of the extended-PK IL-2 is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

In certain embodiments, the extended-PK group is optionally fused to IL-2 via a linker. Linkers suitable for fusing the extended-PK group to IL-2 are well known in the art, and are disclosed in, e.g., US2010/0210511 US2010/0179094, and US2012/0094909, which are herein incorporated by reference in its entirety. Exemplary linkers include gly-ser polypeptide linkers, glycine-proline polypeptide linkers, and proline-alanine polypeptide linkers. In certain embodiments, the linker is a gly-ser polypeptide linker, i.e., a peptide that consists of glycine and serine residues.

Exemplary gly-ser polypeptide linkers comprise the amino acid sequence Ser (Gly₄Ser)n, wherein n=1, 2, 3, 4, 5, 6, 7, 8, 9, or 10 (SEQ ID NO:10). Another exemplary gly-ser polypeptide linker comprises the amino acid sequence Ser (Gly₄Ser)n, wherein n=1, 2, 3, 4, 5, 6, 7, 8, 9, or 10 (SEQ ID NO:10). Another exemplary gly-ser polypeptide linker comprises (Gly₄Ser)n, wherein n=1, 2, 3, 4, 5, 6, 7, 8, 9, or 10 (SEQ ID NO: 11). Another exemplary gly-ser polypeptide linker comprises (Gly₃Ser)n, wherein n=1, 2, 3, 4, 5, 6, 7, 8, 9, or 10 (SEQ ID NO:12).

### Formulations

When an IL-2 therapeutic composition of the present disclosure is administered alone or in combination with other drugs, it is used in form of solid preparation or liquid preparation for oral administration, sustained-release preparation or controlled-release preparation for oral administration, or injection, external preparation, inhalant, suppository or the like for parenteral administration.

Examples of the solid preparation for oral administration include tablets, pills, capsules, powders, granules and the like, and examples of the capsules include hard capsules, soft capsules and the like.

In case of the solid preparation, the IL-2 therapeutic compositions of the present disclosure may be used as it is or by mixing with any one of an excipient (e.g., lactose, mannitol, glucose, microcrystalline cellulose and starch, etc.), binder (e.g., hydroxylpropylcellulose, polyvinylpyrrolidone and magnesium aluminometasilicate, etc.), disintegrant (e.g., calcium fibrin glycolate etc.), lubricant (e.g., magnesium stearate etc.), stabilizer, solubilizer (e.g., glutamic acid and aspartic acid, etc.) and the like so as to be formulated according to conventional methods. If necessary, it may be coated with a coating agent (e.g., sucrose, gelatin, hydroxypropylcellulose and hydroxypropylmethylcellulose phthalate, etc.), or may be coated with two or more layers. Further, it may also be contained in a capsule made of a substance which is easily absorbed by the body, such as gelatin.

The liquid preparation for oral administration may contain, if necessary, any one or more of kinds of pharmaceutically acceptable aqueous solution, suspension, emulsion, syrup, elixir and the like. Furthermore, this liquid preparation may further contain any one or more of kinds of a wetting agent, sweetening agent, flavoring agent, aromatic agent, preservative, buffering agent or the like.

The sustained-release preparation for oral administration may contain may also contain a binder and thickener, in addition to a sustained-release base agent, and examples of thereof include a gum arabic, agar, polyvinylpyrrolidone, sodium alginate, propylene glycol alginate, carboxyvinyl polymer, carboxymethyl cellulose, sodium carboxymethyl cellulose, guar gum, gelatin, hydroxypropyl methyl cellulose, hydroxypropyl cellulose, polyvinyl alcohol, methyl cellulose, hydroxyethyl methyl cellulose or the like.

If using the injection or transfusion for drip infusion, the injection or transfusion may be in any form of aqueous solution, suspension or emulsion, and may be formulated as a solid formulation with a pharmaceutically acceptable carrier so that it can be dissolved, suspended or emulsified by adding a solvent when needed. As a solvent which is used in the injection or transfusion for drip infusion, for example, distilled water for injection, physiological saline, glucose solution and isotonic solution (e.g., a solution of sodium chloride, potassium chloride, glycerin, mannitol, sorbitol, boric acid, borax or propylene glycol, etc.), and the like can be used.

Herein, examples of the pharmaceutically acceptable carrier include a stabilizer, solubilizer, suspending agent, emulsifier, soothing agent, buffer, preservative, antiseptic agent, pH adjuster, antioxidant and the like. As the stabilizer, for example, various amino acids, albumin, globulin, gelatin, mannitol, glucose, dextran, ethylene glycol, propylene glycol, polyethylene glycol, ascorbic acid, sodium hydrogen sulfite, sodium thiosulfate, sodium edetate, sodium citrate, dibutylhydroxytoluene or the like can be used. As the solubilizer, for example, alcohol (e.g., ethanol etc.), polyalcohol (e.g., propylene glycol and polyethylene glycol, etc.), nonionic surfactant (e.g., Polysorbate 20 (registered trademark), Polysorbate 80 (registered trademark) and HCO-50, etc.), etc.) or the like can be used. As the suspending agent, for example, glyceryl monostearate, aluminium monostearate, methyl cellulose, carboxymethyl cellulose, hydroxymethyl cellulose, sodium lauryl sulfate or the like can be used. As the emulsifier, for example, gum arabic, sodium alginate, tragacanth or the like can be used. As the soothing agent, for example, benzyl alcohol, chlorobutanol, sorbitol or the like can be used. As the buffer, phosphate buffer, acetate buffer, borate buffer, carbonate buffer, citrate buffer, Tris buffer, glutamate buffer, epsilon aminocaproate buffer or the like can be used. As the preservative, for example, methyl paraoxybenzoate, ethyl paraoxybenzoate, propyl paraoxybenzoate, butyl paraoxybenzoate, chlorobutanol, benzyl alcohol, benzalkonium chloride, dehydro sodium acetate, sodium edetate, boric acid, borax or the like can be used. As the antiseptic agent, for example, benzalkonium chloride, paraoxybenzoic acid, chlorobutanol or the like can be used. As the pH adjuster, for example, hydrochloric acid, sodium hydroxide, phosphoric acid, acetic acid or the like can be used. As the antioxidant, for example, (1) a water-soluble antioxidant such as ascorbic acid, cysteine hydrochloride, sodium bisulfate, sodium metabisulfite, sodium sulfite and the like, (2) an oil-soluble antioxidant such as ascorbyl palmitate, butylated hydroxyanisole, butylated hydroxytoluene, lecithin, propyl gallate, a-tocopherol and the like, or (3) a metal chelating agent such as citric acid, ethylenediaminetetraacetic acid, sorbitol, tartaric acid, phosphoric acid and the like can be used.

The injection or transfusion for drip infusion can be produced by sterilizing it at the final step or by aseptic operation methods, for example, filtering with a filter or the like, followed by filling a sterile container. Alternatively, the injection or transfusion for drip infusion may be used by dissolving a sterile powder obtained by vacuum drying and freeze-drying (which may contain a powder of pharmaceutically acceptable carrier) in a suitable solvent before use.

The external preparation for parenteral administration can be used in a form of a propellant, inhalant, spray, aerosol, ointment, gel, cream, poultice, patch, liniment, nasal drop or the like, which is prepared by publically known methods or in usually used formulation.

The propellant, inhalant and spray may contain a stabilizer such as sodium bisulfite, other than commonly used diluents and buffer giving isotonicity, for example, an isotonic agent such as sodium chloride, sodium citrate and citric acid. The method for producing the spray is described in, for example, U.S. Pat. Nos. 2,868,691 and 3,095,355, in detail.

Examples of the inhalants include an aerosol agent, inhalant powder or inhalant liquid, and the inhalant liquid may be used in a form of being dissolved or suspended in water or other appropriate mediums before use.

These inhalants can be manufactured according to publically known methods, for example, if it is an inhalant liquid, it can be prepared by appropriately selecting a preservative (e.g., benzalkonium chloride and paraben, etc.), coloring agent, buffer (e.g., sodium phosphate and sodium acetate, etc.), isotonicity agent (e.g., sodium chloride and concentrated glycerin, etc.), thickener (e.g., carboxyvinyl polymer etc.), absorption enhancer or the like, if necessary. If it is an inhalant powder, it can be prepared by appropriately selecting a lubricant (e.g., stearic acid and salt thereof, etc.), binder (e.g., starch and dextrin, etc.), excipient (e.g., lactose and cellulose, etc.), coloring agent, preservative (e.g., benzalkonium chloride and paraben, etc.), absorption enhancer or the like, if necessary.

When administering the inhalant liquid, a nebulizer (e.g., atomizer and nebulizer, etc.) is usually used while when administering the inhalant powder, an inhaler for a powdered medicine is usually used.

The ointment is prepared in a publically known or commonly used formulation, for example, can be prepared by mixing the IL-2 therapeutic compositions of the present disclosure in base. An ointment base can be selected from publically known or commonly used ones, which is used by mixing with, for example, one or more kinds selected from a higher fatty acid or higher fatty acid ester (e.g., adipic acid, myristic acid, palmitic acid, stearic acid, oleic acid, adipic acid ester, myristic acid ester, palmitic acid ester, stearic acid ester and oleic acid ester, etc.), waxes (e.g., beeswax, whale wax and ceresin, etc.), surfactant (e.g., polyoxyethylene alkyl ether phosphate etc.), higher alcohol (e.g., cetanol, stearyl alcohol and cetostearyl alcohol, etc.), silicone oil (e.g., dimethyl polysiloxane etc.), hydrocarbons (e.g., hydrophilic petrolatum, white petrolatum, purified lanolin and liquid paraffin, etc.), glycols (e.g., ethylene glycol, diethylene glycol, propylene glycol, polyethylene glycol and macrogol, etc.), vegetable oil (e.g., castor oil, olive oil, sesame oil and turpentine oil, etc.), animal oil (e.g., mink oil, egg yolk oil, squalane and squalene, etc.), water, absorption promoter and anti-rash agent. Furthermore, it may contain a moisturizing agent, preservative, stabilizer, antioxidant, flavoring agent or the like.

The gel is prepared in a publically known or commonly used formulation, for example, can be prepared by melting the IL-2 therapeutic compositions of the present disclosure in base. A gel base is selected from publically known or commonly used ones, which is used by mixing with, for example, one or more kinds selected from a lower alcohol (e.g., ethanol and isopropyl alcohol, etc.), gelling agent (e.g., carboxymethyl cellulose, hydroxyethyl cellulose, hydroxypropyl cellulose and ethyl cellulose, etc.), neutralizing agent (e.g., triethanolamine and diisopropanolamine, etc.), surfactant (e.g., polyethylene glycol monostearate etc.), gums, water, absorption promoter and anti-rash agent. Furthermore, it may contain a preservative, antioxidant, flavoring agent or the like.

The cream is prepared in a publically known or commonly used formulation, for example, can be prepared by melting or emulsifying the IL-2 therapeutic compositions of the present disclosure in base. A cream base is selected from publically known or commonly used ones, which is used by mixing with, for example, one or more kinds selected from a higher fatty acid ester, lower alcohol, hydrocarbons, polyhydric alcohol (e.g., propylene glycol and 1,3-butylene glycol, etc.), higher alcohol (e.g., 2-hexyldecanol and cetanol, etc.), emulsifier (e.g., polyoxyethylene alkyl ethers and fatty acid esters, etc.), water, absorption promoter and anti-rash agent. Furthermore, it may contain a preservative, antioxidant, flavoring agent or the like.

The poultice is prepared in a publically known or commonly used formulation, for example, can be prepared by melting the IL-2 therapeutic compositions of the present disclosure in base and spreading and coating it on a support as a kneaded product. A poultice base is selected from publically known or commonly used ones, which is used by mixing with, for example, one or more kinds selected from a thickener (e.g., polyacrylic acid, polyvinylpyrrolidone, arabic gum, starch, gelatin and methylcellulose, etc.), wetting agent (e.g., urea, glycerin and propylene glycol, etc.), filler (e.g., kaolin, zinc oxide, talc, calcium and magnesium, etc.), water, solubilizing agent, tackifier and anti-rash agent. Furthermore, it may contain a preservative, antioxidant, flavoring agent or the like.

The patch is prepared in a publically known or commonly used formulation, for example, can be prepared by melting the IL-2 therapeutic compositions of the present disclosure in base and spreading and coating it on a support. A patch base is selected from publically known or commonly used ones, which is used by mixing with, for example, one or more kinds selected from a polymer base, fats and oils, higher fatty acid, tackifier and anti-rash agent. Furthermore, it may contain a preservative, antioxidant, flavoring agent or the like.

The liniment is prepared in a publically known or commonly used formulation, for example, can be prepared by dissolving, suspending or emulsifying the IL-2 therapeutic compositions of the present disclosure in one or more of water, an alcohol (e.g., ethanol and polyethylene glycol, etc.), higher fatty acid, glycerin, soap, emulsifier, suspending agent and the like. Furthermore, it may contain a preservative, antioxidant, flavoring agent or the like. Examples of other compositions for parenteral administration include a suppository for rectal administration and pessary for vaginal administration which contain the IL-2 therapeutic compositions of the present disclosure and are prescribed by conventional methods.

### Modes of Administration and Effective Dosages

Any method known to those in the art for contacting a cell, organ or tissue with one or more IL-2 therapeutic compositions disclosed herein may be employed. Suitable methods include in vitro, ex vivo, or in vivo methods. In vivo methods typically include the administration of one or more IL-2 therapeutic compositions to a mammal, suitably a human. When used in vivo for therapy, the one or more IL-2 therapeutic compositions described herein are administered to the subject in effective amounts (i.e., amounts that have desired therapeutic effect). The dose and dosage regimen will depend upon the degree of the disease state of the subject, the characteristics of the particular IL-2 therapeutic composition used, e.g., its therapeutic index, and the subject's history.

The effective amount may be determined during pre-clinical trials and clinical trials by methods familiar to physicians and clinicians. An effective amount of one or more IL-2 therapeutic compositions useful in the methods may be administered to a mammal in need thereof by any of a number of well-known methods for administering pharmaceutical compounds. The IL-2 therapeutic compositions may be administered systemically or locally.

The one or more IL-2 therapeutic compositions described herein can be incorporated into pharmaceutical compositions for administration, singly or in combination, to a subject for the treatment or prevention of melanoma. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intratumoral (e.g., intralesional injection), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more IL-2 therapeutic compositions disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34 (7-8): 915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly a-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8): 915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4 (3): 201-9 (1994); and Gregoriadis, “Engineering Liposomes for Drug Delivery: Progress and Problems,” Trends Biotechnol., 13 (12): 527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more IL-2 therapeutic compositions disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more IL-2 therapeutic composition concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more IL-2 therapeutic compositions may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

Methods for Detecting Polynucleotides Associated with Positive or Negative Responsiveness to IL-2 Therapy

Polynucleotides associated with responsiveness to IL-2 therapy may be detected by a variety of methods known in the art. Non-limiting examples of detection methods are described below. The detection assays in the methods of the present technology may include purified or isolated DNA (genomic or cDNA), RNA or protein or the detection step may be performed directly from a biological sample without the need for further DNA, RNA or protein purification/isolation.

### Nucleic Acid Amplification and or Detection

Polynucleotides associated with responsiveness to IL-2 therapy can be detected by the use of nucleic acid amplification techniques that are well known in the art. The starting material may be genomic DNA, cDNA, RNA or mRNA. Nucleic acid amplification can be linear or exponential. Specific variants may be detected by the use of amplification methods with the aid of oligonucleotide primers or probes designed to interact with or hybridize to a particular target sequence in a specific manner, thus amplifying only the target variant.

Non-limiting examples of nucleic acid amplification techniques include polymerase chain reaction (PCR), real-time quantitative PCR (qPCR), digital PCR (dPCR), reverse transcriptase polymerase chain reaction (RT-PCR), nested PCR, ligase chain reaction (see Abravaya, K. et al., Nucleic Acids Res. (1995), 23:675-682), branched DNA signal amplification (see Urdea, M. S. et al., AIDS (1993), 7 (suppl 2): S11-S14), amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, next-generation sequencing technology or other sequence replication assays or signal amplification assays.

### Primers:

Oligonucleotide primers for use in amplification methods can be designed according to general guidance well known in the art as described herein, as well as with specific requirements as described herein for each step of the particular methods described. In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length.

Tₘ of a polynucleotide affects its hybridization to another polynucleotide (e.g., the annealing of an oligonucleotide primer to a template polynucleotide). In certain embodiments of the disclosed methods, the oligonucleotide primer used in various steps selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In certain embodiments, 100% complementarity exists.

### Probes:

Probes are capable of hybridizing to at least a portion of the nucleic acid of interest or a reference nucleic acid (i.e., wild-type sequence). Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, or about 100 nucleotides long. However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, or about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif, 1987); Young and Davis, PNAS. 80:1194 (1983).

Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29: E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl) aminonaphthalene-1-sulfonic acid (EDANS); 4-amino-N-[3-vinylsulfonyl)phenyl] naphthalimide-3,5 disulfonate (Lucifer Yellow VS); N-(4-anilino-1-naphthyl) maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo) benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl) amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSYR 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Detectably labeled probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detectably labeled probes can also include two probes, wherein for example a fluorophore is on one probe, and a quencher is on the other probe, wherein hybridization of the two probes together on a target quenches the signal, or wherein hybridization on the target alters the signal signature via a change in fluorescence.

In some embodiments, interchelating labels such as ethidium bromide, SYBR® Green I (Molecular Probes), and PicoGreen® (Molecular Probes) are used, thereby allowing visualization in real-time, or at the end point, of an amplification product in the absence of a detector probe. In some embodiments, real-time visualization may involve the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

Primers or probes may be designed to selectively hybridize to any portion of a nucleic acid sequence encoding a MHC class I polypeptide. Exemplary nucleic acid sequences of the human orthologs of these genes are provided below:

Homo sapiens major histocompatibility complex, class I,
A (HLA-A), transcript variant 1 (A*03:01:01:01), mRNA
(NCBI Reference Sequence: NM_002116.8)
(SEQ ID NO: 3)
AGATTCTCCCCAGACGCCGAGGATGGCCGTCATGGCGCCCCGAACCCTCCTCCTGCTACTCTCGGGGGCC
CTGGCCCTGACCCAGACCTGGGCGGGCTCCCACTCCATGAGGTATTTCTTCACATCCGTGTCCCGGCCCG
GCCGCGGGGAGCCCCGCTTCATCGCCGTGGGCTACGTGGACGACACGCAGTTCGTGCGGTTCGACAGCGA
CGCCGCGAGCCAGAGGATGGAGCCGCGGGCGCCGTGGATAGAGCAGGAGGGGCCGGAGTATTGGGACCAG
GAGACACGGAATGTGAAGGCCCAGTCACAGACTGACCGAGTGGACCTGGGGACCCTGCGCGGCTACTACA
ACCAGAGCGAGGCCGGTTCTCACACCATCCAGATAATGTATGGCTGCGACGTGGGGTCGGACGGGCGCTT
CCTCCGCGGGTACCGGCAGGACGCCTACGACGGCAAGGATTACATCGCCCTGAACGAGGACCTGCGCTCT
TGGACCGCGGCGGACATGGCGGCTCAGATCACCAAGCGCAAGTGGGAGGCGGCCCATGAGGCGGAGCAGT
TGAGAGCCTACCTGGATGGCACGTGCGTGGAGTGGCTCCGCAGATACCTGGAGAACGGGAAGGAGACGCT
GCAGCGCACGGACCCCCCCAAGACACATATGACCCACCACCCCATCTCTGACCATGAGGCCACCCTGAGG
TGCTGGGCCCTGGGCTTCTACCCTGCGGAGATCACACTGACCTGGCAGCGGGATGGGGAGGACCAGACCC
AGGACACGGAGCTCGTGGAGACCAGGCCTGCAGGGGATGGAACCTTCCAGAAGTGGGCGGCTGTGGTGGT
GCCTTCTGGAGAGGAGCAGAGATACACCTGCCATGTGCAGCATGAGGGTCTGCCCAAGCCCCTCACCCTG
AGATGGGAGCTGTCTTCCCAGCCCACCATCCCCATCGTGGGCATCATTGCTGGCCTGGTTCTCCTTGGAG
CTGTGATCACTGGAGCTGTGGTCGCTGCCGTGATGTGGAGGAGGAAGAGCTCAGATAGAAAAGGAGGGAG
TTACACTCAGGCTGCAAGCAGTGACAGTGCCCAGGGCTCTGATGTGTCCCTCACAGCTTGTAAAGTGTGA
GACAGCTGCCTTGTGTGGGACTGAGAGGCAAGAGTTGTTCCTGCCCTTCCCTTTGTGACTTGAAGAACCC
TGACTTTGTTTCTGCAAAGGCACCTGCATGTGTCTGTGTTCGTGTAGGCATAATGTGAGGAGGTGGGGAG
ACCACCCCACCCCCATGTCCACCATGACCCTCTTCCCACGCTGACCTGTGCTCCCTCCCCAATCATCTTT
CCTGTTCCAGAGAGGTGGGGCTGAGGTGTCTCCATCTCTGTCTCAACTTCATGGTGCACTGAGCTGTAAC
TTCTTCCTTCCCTATTAAAATTAGAACCTTAGTATAAATTTACTTTCTCAAATTCTTGCCATGAGAGGTT
GATGAGTTAATTAAAGGAGAAGATTCCTAAAATTTGAGAGACAAAATAAATGGAAGACATGAGAA
Homo sapiens major histocompatibility complex, class I,
A (HLA-A), transcript variant 2 (A*01:01:01:01), mRNA
(NCBI Reference Sequence: NM_001242758.1)
(SEQ ID NO: 4)
GAGAAGCCAATCAGTGTCGTCGCGGTCGCTGTTCTAAAGTCCGCACGCACCCACCGGGACTCAGATTCTC
CCCAGACGCCGAGGATGGCCGTCATGGCGCCCCGAACCCTCCTCCTGCTACTCTCGGGGGCCCTGGCCCT
GACCCAGACCTGGGCGGGCTCCCACTCCATGAGGTATTTCTTCACATCCGTGTCCCGGCCCGGCCGCGGG
GAGCCCCGCTTCATCGCCGTGGGCTACGTGGACGACACGCAGTTCGTGCGGTTCGACAGCGACGCCGCGA
GCCAGAAGATGGAGCCGCGGGCGCCGTGGATAGAGCAGGAGGGGCCGGAGTATTGGGACCAGGAGACACG
GAATATGAAGGCCCACTCACAGACTGACCGAGCGAACCTGGGGACCCTGCGCGGCTACTACAACCAGAGC
GAGGACGGTTCTCACACCATCCAGATAATGTATGGCTGCGACGTGGGGCCGGACGGGCGCTTCCTCCGCG
GGTACCGGCAGGACGCCTACGACGGCAAGGATTACATCGCCCTGAACGAGGACCTGCGCTCTTGGACCGC
GGCGGACATGGCAGCTCAGATCACCAAGCGCAAGTGGGAGGCGGTCCATGCGGCGGAGCAGCGGAGAGTC
TACCTGGAGGGCCGGTGCGTGGACGGGCTCCGCAGATACCTGGAGAACGGGAAGGAGACGCTGCAGCGCA
CGGACCCCCCCAAGACACATATGACCCACCACCCCATCTCTGACCATGAGGCCACCCTGAGGTGCTGGGC
CCTGGGCTTCTACCCTGCGGAGATCACACTGACCTGGCAGCGGGATGGGGAGGACCAGACCCAGGACACG
GAGCTCGTGGAGACCAGGCCTGCAGGGGATGGAACCTTCCAGAAGTGGGCGGCTGTGGTGGTGCCTTCTG
GAGAGGAGCAGAGATACACCTGCCATGTGCAGCATGAGGGTCTGCCCAAGCCCCTCACCCTGAGATGGGA
GCTGTCTTCCCAGCCCACCATCCCCATCGTGGGCATCATTGCTGGCCTGGTTCTCCTTGGAGCTGTGATC
ACTGGAGCTGTGGTCGCTGCCGTGATGTGGAGGAGGAAGAGCTCAGATAGAAAAGGAGGGAGTTACACTC
AGGCTGCAAGCAGTGACAGTGCCCAGGGCTCTGATGTGTCTCTCACAGCTTGTAAAGTGTGAGACAGCTG
CCTTGTGTGGGACTGAGAGGCAAGAGTTGTTCCTGCCCTTCCCTTTGTGACTTGAAGAACCCTGACTTTG
TTTCTGCAAAGGCACCTGCATGTGTCTGTGTTCGTGTAGGCATAATGTGAGGAGGTGGGGAGAGCACCCC
ACCCCCATGTCCACCATGACCCTCTTCCCACGCTGACCTGTGCTCCCTCTCCAATCATCTTTCCTGTTCC
AGAGAGGTGGGGCTGAGGTGTCTCCATCTCTGTCTCAACTTCATGGTGCACTGAGCTGTAACTTCTTCCT
TCCCTATTAAAATTAGAACCTGAGTATAAATTTACTTTCTCAAATTCTTGCCATGAGAGGTTGATGAGTT
AATTAAAGGAGAAGATTCCTAAAATTTGAGAGACAAAATTAATGGAACGCATGAGAACCTTCCAGAGTCC
A
Homo sapiens major histocompatibility complex, class I,
B (HLA-B), mRNA (NCBI Reference Sequence: NM_005514.8)
(SEQ ID NO: 5)
AGAGTCTCCTCAGACGCCGAGATGCTGGTCATGGCGCCCCGAACCGTCCTCCTGCTGCTCTCGGCGGCCC
TGGCCCTGACCGAGACCTGGGCCGGCTCCCACTCCATGAGGTATTTCTACACCTCCGTGTCCCGGCCCGG
CCGCGGGGAGCCCCGCTTCATCTCAGTGGGCTACGTGGACGACACCCAGTTCGTGAGGTTCGACAGCGAC
GCCGCGAGTCCGAGAGAGGAGCCGCGGGCGCCGTGGATAGAGCAGGAGGGGCCGGAGTATTGGGACCGGA
ACACACAGATCTACAAGGCCCAGGCACAGACTGACCGAGAGAGCCTGCGGAACCTGCGCGGCTACTACAA
CCAGAGCGAGGCCGGGTCTCACACCCTCCAGAGCATGTACGGCTGCGACGTGGGGCCGGACGGGCGCCTC
CTCCGCGGGCATGACCAGTACGCCTACGACGGCAAGGATTACATCGCCCTGAACGAGGACCTGCGCTCCT
GGACCGCCGCGGACACGGCGGCTCAGATCACCCAGCGCAAGTGGGAGGCGGCCCGTGAGGCGGAGCAGCG
GAGAGCCTACCTGGAGGGCGAGTGCGTGGAGTGGCTCCGCAGATACCTGGAGAACGGGAAGGACAAGCTG
GAGCGCGCTGACCCCCCAAAGACACACGTGACCCACCACCCCATCTCTGACCATGAGGCCACCCTGAGGT
GCTGGGCCCTGGGTTTCTACCCTGCGGAGATCACACTGACCTGGCAGCGGGATGGCGAGGACCAAACTCA
GGACACTGAGCTTGTGGAGACCAGACCAGCAGGAGATAGAACCTTCCAGAAGTGGGCAGCTGTGGTGGTG
CCTTCTGGAGAAGAGCAGAGATACACATGCCATGTACAGCATGAGGGGCTGCCGAAGCCCCTCACCCTGA
GATGGGAGCCGTCTTCCCAGTCCACCGTCCCCATCGTGGGCATTGTTGCTGGCCTGGCTGTCCTAGCAGT
TGTGGTCATCGGAGCTGTGGTCGCTGCTGTGATGTGTAGGAGGAAGAGTTCAGGTGGAAAAGGAGGGAGC
TACTCTCAGGCTGCGTGCAGCGACAGTGCCCAGGGCTCTGATGTGTCTCTCACAGCTTGAAAAGCCTGAG
ACAGCTGTCTTGTGAGGGACTGAGATGCAGGATTTCTTCACGCCTCCCCTTTGTGACTTCAAGAGCCTCT
GGCATCTCTTTCTGCAAAGGCACCTGAATGTGTCTGCGTCCCTGTTAGCATAATGTGAGGAGGTGGAGAG
ACAGCCCACCCTTGTGTCCACTGTGACCCCTGTTCCCATGCTGACCTGTGTTTCCTCCCCAGTCATCTTT
CTTGTTCCAGAGAGGTGGGGCTGGATGTCTCCATCTCTGTCTCAACTTTACGTGCACTGAGCTGCAACTT
CTTACTTCCCTACTGAAAATAAGAATCTGAATATAAATTTGTTTTCTCAAATATTTGCTATGAGAGGTTG
ATGGATTAATTAAATAAGTCAATTCCTGGAATTTGAGAGAGCAAATAAAGACCTGAGAACCTTCCA
Homo sapiens major histocompatibility complex, class I,
C (HLA-C), transcript variant 1, mRNA (NCBI Reference
Sequence: NM_002117.6)
(SEQ ID NO: 6)
ACATTCTCCCCAGAGGCCGAGATGCGGGTCATGGCGCCCCGAGCCCTCCTCCTGCTGCTCTCGGGAGGCC
TGGCCCTGACCGAGACCTGGGCCTGCTCCCACTCCATGAGGTATTTCGACACCGCCGTGTCCCGGCCCGG
CCGCGGAGAGCCCCGCTTCATCTCAGTGGGCTACGTGGACGACACGCAGTTCGTGCGGTTCGACAGCGAC
GCCGCGAGTCCGAGAGGGGAGCCGCGGGCGCCGTGGGTGGAGCAGGAGGGGCCGGAGTATTGGGACCGGG
AGACACAGAAGTACAAGCGCCAGGCACAGGCTGACCGAGTGAGCCTGCGGAACCTGCGCGGCTACTACAA
CCAGAGCGAGGACGGGTCTCACACCCTCCAGAGGATGTCTGGCTGCGACCTGGGGCCCGACGGGCGCCTC
CTCCGCGGGTATGACCAGTCCGCCTACGACGGCAAGGATTACATCGCCCTGAACGAGGACCTGCGCTCCT
GGACCGCCGCGGACACCGCGGCTCAGATCACCCAGCGCAAGTTGGAGGCGGCCCGTGCGGCGGAGCAGCT
GAGAGCCTACCTGGAGGGCACGTGCGTGGAGTGGCTCCGCAGATACCTGGAGAACGGGAAGGAGACGCTG
CAGCGCGCAGAACCCCCAAAGACACACGTGACCCACCACCCCCTCTCTGACCATGAGGCCACCCTGAGGT
GCTGGGCCCTGGGCTTCTACCCTGCGGAGATCACACTGACCTGGCAGCGGGATGGGGAGGACCAGACCCA
GGACACCGAGCTTGTGGAGACCAGGCCAGCAGGAGATGGAACCTTCCAGAAGTGGGCAGCTGTGGTGGTG
CCTTCTGGACAAGAGCAGAGATACACGTGCCATATGCAGCACGAGGGGCTGCAAGAGCCCCTCACCCTGA
GCTGGGAGCCATCTTCCCAGCCCACCATCCCCATCATGGGCATCGTTGCTGGCCTGGCTGTCCTGGTTGT
CCTAGCTGTCCTTGGAGCTGTGGTCACCGCTATGATGTGTAGGAGGAAGAGCTCAGGTGGAAAAGGAGGG
AGCTGCTCTCAGGCTGCGTGCAGCAACAGTGCCCAGGGCTCTGATGAGTCTCTCATCACTTGTAAAGCCT
GAGACAGCTGCCTGTGTGGGACTGAGATGCAGGATTTCTTCACACCTCTCCTTTGTGACTTCAAGAGCCT
CTGGCATCTCTTTCTGCAAAGGCACCTGAATGTGTCTGCGTTCCTGTTAGCATAATGTGAGGAGGTGGAG
AGACAGCCCACCCCCGTGTCCACCGTGACCCCTGTCCCCACACTGACCTGTGTTCCCTCCCCGATCATCT
TTCCTGTTCCAGAGAGGTGGGGCTGGATGTCTCCATCTCTGTCTCAAATTCATGGTGCACTGAGCTGCAA
CTTCTTACTTCCCTAATGAAGTTAAGAACCTGAATATAAATTTGTGTTCTCAAATATTTGCTATGAAGCG
TTGATGGATTAATTAAATAAGTCAATTCCTAGAAGTTGAGAGAGCAAATAAAGACCTGAGAACCTTCCAG
AA
Homo sapiens major histocompatibility complex, class I,
C (HLA-C), transcript variant 2, mRNA (NCBI Reference
Sequence: NM_001243042.1)
(SEQ ID NO: 7)
TCCGCAGTCCCGGTTCTAAAGTCCCCAGTCACCCACCCGGACTCACATTCTCCCCAGAGGCCGAGATGCG
GGTCATGGCGCCCCGAGCCCTCCTCCTGCTGCTCTCGGGAGGCCTGGCCCTGACCGAGACCTGGGCCTGC
TCCCACTCCATGAGGTATTTCGACACCGCCGTGTCCCGGCCCGGCCGCGGAGAGCCCCGCTTCATCTCAG
TGGGCTACGTGGACGACACGCAGTTCGTGCGGTTCGACAGCGACGCCGCGAGTCCGAGAGGGGAGCCGCG
GGCGCCGTGGGTGGAGCAGGAGGGGCCGGAGTATTGGGACCGGGAGACACAGAACTACAAGCGCCAGGCA
CAGGCTGACCGAGTGAGCCTGCGGAACCTGCGCGGCTACTACAACCAGAGCGAGGACGGGTCTCACACCC
TCCAGAGGATGTATGGCTGCGACCTGGGGCCCGACGGGCGCCTCCTCCGCGGGTATGACCAGTCCGCCTA
CGACGGCAAGGATTACATCGCCCTGAACGAGGACCTGCGCTCCTGGACCGCCGCGGACACCGCGGCTCAG
ATCACCCAGCGCAAGTTGGAGGCGGCCCGTGCGGCGGAGCAGCTGAGAGCCTACCTGGAGGGCACGTGCG
TGGAGTGGCTCCGCAGATACCTGGAGAACGGGAAGGAGACGCTGCAGCGCGCAGAACCCCCAAAGACACA
CGTGACCCACCACCCCCTCTCTGACCATGAGGCCACCCTGAGGTGCTGGGCCCTGGGCTTCTACCCTGCG
GAGATCACACTGACCTGGCAGCGGGATGGGGAGGACCAGACCCAGGACACCGAGCTTGTGGAGACCAGGC
CAGCAGGAGATGGAACCTTCCAGAAGTGGGCAGCTGTGGTGGTGCCTTCTGGACAAGAGCAGAGATACAC
GTGCCATATGCAGCACGAGGGGCTGCAAGAGCCCCTCACCCTGAGCTGGGAGCCATCTTCCCAGCCCACC
ATCCCCATCATGGGCATCGTTGCTGGCCTGGCTGTCCTGGTTGTCCTAGCTGTCCTTGGAGCTGTGGTCA
CCGCTATGATGTGTAGGAGGAAGAGCTCAGGTGGAAAAGGAGGGAGCTGCTCTCAGGCTGCGTGCAGCAA
CAGTGCCCAGGGCTCTGATGAGTCTCTCATCACTTGTAAAGCCTGAGACAGCTGCCTGTGTGGGACTGAG
ATGCAGGATTTCTTCACACCTCTCCTTTGTGACTTCAAGAGCCTCTGGCATCTCTTTCTGCAAAGGCGTC
TGAATGTGTCTGCGTTCCTGTTAGCATAATGTGAGGAGGTGGAGAGACAGCCCACCCCCGTGTCCACCGT
GACCCCTGTCCCCACACTGACCTGTGTTCCCTCCCCGATCATCTTTCCTGTTCCAGAGAGGTGGGGCTGG
ATGTCTCCATCTCTGTCTCAAATTCATGGTGCACTGAGCTGCAACTTCTTACTTCCCTAATGAAGTTAAG
AACCTGAATATAAATTTGTGTTCTCAAATATTTGCTATGAAGCGTTGATGGATTAATTAAATAAGTCAAT
TCCTAGAAGTTGAGAGAGCAAATAAAGACCTGAGAACCTTCCAGAA
Homo sapiens beta-2-microglobulin (B2M), mRNA (NCBI
Reference Sequence: NM_004048.4)
(SEQ ID NO: 8)
ATTCCTGAAGCTGACAGCATTCGGGCCGAGATGTCTCGCTCCGTGGCCTTAGCTGTGCTCGCGCTACTCT
CTCTTTCTGGCCTGGAGGCTATCCAGCGTACTCCAAAGATTCAGGTTTACTCACGTCATCCAGCAGAGAA
TGGAAAGTCAAATTTCCTGAATTGCTATGTGTCTGGGTTTCATCCATCCGACATTGAAGTTGACTTACTG
AAGAATGGAGAGAGAATTGAAAAAGTGGAGCATTCAGACTTGTCTTTCAGCAAGGACTGGTCTTTCTATC
TCTTGTACTACACTGAATTCACCCCCACTGAAAAAGATGAGTATGCCTGCCGTGTGAACCATGTGACTTT
GTCACAGCCCAAGATAGTTAAGTGGGATCGAGACATGTAAGCAGCATCATGGAGGTTTGAAGATGCCGCA
TTTGGATTGGATGAATTCCAAATTCTGCTTGCTTGCTTTTTAATATTGATATGCTTATACACTTACACTT
TATGCACAAAATGTAGGGTTATAATAATGTTAACATGGACATGATCTTCTTTATAATTCTACTTTGAGTG
CTGTCTCCATGTTTGATGTATCTGAGCAGGTTGCTCCACAGGTAGCTCTAGGAGGGCTGGCAACTTAGAG
GTGGGGAGCAGAGAATTCTCTTATCCAACATCAACATCTTGGTCAGATTTGAACTCTTCAATCTCTTGCA
CTCAAAGCTTGTTAAGATAGTTAAGCGTGCATAAGTTAACTTCCAATTTACATACTCTGCTTAGAATTTG
GGGGAAAATTTAGAAATATAATTGACAGGATTATTGGAAATTTGTTATAATGAATGAAACATTTTGTCAT
ATAAGATTCATATTTACTTCTTATACATTTGATAAAGTAAGGCATGGTTGTGGTTAATCTGGTTTATTTT
TGTTCCACAAGTTAAATAAATCATAAAACTTGA

Primers or probes can be designed so that they hybridize under stringent conditions to mutant nucleotide sequences of at least one of B2M, HLA-A, HLA-B, and HLA-C, but not to the respective wild-type nucleotide sequences. Primers or probes can also be prepared that are complementary and specific for the wild-type nucleotide sequence of at least one of B2M, HLA-A, HLA-B, and HLA-C, but not to any one of the corresponding mutant nucleotide sequences.

In some embodiments, detection can occur through any of a variety of mobility dependent analytical techniques based on the differential rates of migration between different nucleic acid sequences. Exemplary mobility-dependent analysis techniques include electrophoresis, chromatography, mass spectroscopy, sedimentation, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described in Published PCT Applications WO04/46344 and WO01/92579. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003).

It is also understood that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. In some embodiments, unlabeled reaction products may be detected using mass spectrometry.

### NGS Platforms

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLID sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454TM GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454TM system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added poly A tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLID™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

### Methods for Selecting Melanoma Patients for IL-2 Therapy

In one aspect, the present disclosure provides a method for selecting a melanoma cancer patient for treatment with IL-2 therapy comprising (a) detecting the presence of wild-type polynucleotides in at least one MHC class I gene in a biological sample obtained from the melanoma cancer patient; and (b) administering to the melanoma cancer patient an effective amount of a therapeutic IL-2 composition. The at least one MHC class I gene may be selected from the group consisting of HLA-A, HLA-B, HLA-C, and B2M. In some embodiments, the wild-type polynucleotides are detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). Additionally or alternatively, in some embodiments, the biological sample comprises genomic DNA, cDNA, RNA, and/or mRNA.

In one aspect, the present disclosure provides a method for selecting a melanoma cancer patient for treatment with IL-2 therapy comprising (a) detecting mRNA and/or polypeptide expression levels of at least one MHC class I gene in a biological sample obtained from the melanoma cancer patient that are comparable to a control sample obtained from a healthy subject or a predetermined threshold; and (b) administering to the melanoma cancer patient an effective amount of a therapeutic IL-2 composition. The at least one MHC class I gene may be selected from the group consisting of HLA-A, HLA-B, HLA-C, and B2M. In another aspect, the present disclosure provides a method for treating melanoma in a patient in need thereof comprising administering to the patient an effective amount of a therapeutic IL-2 composition, wherein mRNA and/or polypeptide expression levels of one or more of HLA-A, HLA-B, HLA-C, and B2M in a biological sample obtained from the patient are comparable to a control sample obtained from a healthy subject or a predetermined threshold. Additionally or alternatively, in some embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). Additionally or alternatively, in certain embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

In any of the preceding embodiments of the methods disclosed herein, the biological sample comprises skin tissue, soft tissue, lymph nodes, whole blood, plasma, or serum. The biological sample may comprise tumor cells and/or macrophages. In certain embodiments, the tumor cells exhibit membranous expression of MHC class I genes.

Additionally or alternatively, in some embodiments of the methods disclosed herein, the therapeutic IL-2 composition comprises wild-type human IL-2, a variant human IL-2 or a chimeric IL-2 fusion polypeptide. In certain embodiments, the wild-type human IL-2 or the chimeric IL-2 fusion polypeptide comprises the amino acid sequence of SEQ ID NO: 1 or SEQ ID NO: 2. Additionally or alternatively, in some embodiments, the chimeric IL-2 fusion polypeptide comprises a Fc domain, a serum albumin domain, a polyethylene glycol (PEG) domain, transferrin, a serum albumin binding protein, a serum immunoglobulin binding protein and a fibronectin (Fn)-based scaffold domain. In any and all embodiments of the methods disclosed herein, the therapeutic IL-2 composition is administered systemically, intratumorally or intravenously.

In any and all embodiments of the methods disclosed herein, the effective amount of the therapeutic IL-2 composition is administered as a series of injections. In certain embodiments, the effective amount of the therapeutic IL-2 composition is administered as a series of 2, 3, 4, 5, 6, 7, 8, 9, 10, 11, 12 or more injections. Additionally or alternatively, in some embodiments, the series of injections are administered over an interval of at least 7 days, at least 14 days, at least 21 days, at least 28 days, at least 35 days, at least 42 days, at least 49 days, at least 56 days, at least 63 days, at least 70 days, at least 80 days, at least 90 days, at least 100 days, at least 110 days, at least 120 days, at least 130 days, at least 140 days, at least 150 days, at least 160 days, at least 170 days, at least 180 days, at least 190 days, at least 200 days, at least 210 days, at least 220 days, at least 230 days, at least 240 days, at least 250 days, at least 260 days, at least 270 days, at least 280 days, or at least 290 days.

In any of the foregoing embodiments of the methods disclosed herein, the patient exhibits stage I, stage II, stage III or stage IV melanoma. The melanoma may be superficial spreading, nodular, lentigo maligna, acral lentiginous, amelanotic, or desmoplastic. In some embodiments, the melanoma is metastatic or localized. Additionally or alternatively, in some embodiments, the patient has a high or low tumor mutation burden (TMB). In certain embodiments, the patient is human.

### Kits of the Present Technology

In one aspect, the present disclosure provides kits for performing the methods of the present technology, wherein said kits comprise means for measuring the expression level of one or more MHC Class I genes in a biological sample obtained from a melanoma patient, wherein said means includes a set of reagents specific for the one or more MHC Class I genes, and wherein the reagents are selected from the group consisting of probes, primers and antibodies specific for each of the one or more MHC Class I genes. The one or more MHC class I genes may be selected from the group consisting of HLA-A, HLA-B, HLA-C, and B2M.

The kits may include probes, primers macroarrays or microarrays. For example, the kit may comprise a set of probes as described herein, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. Alternatively the kits of the present disclosure may comprise amplification primers that may be pre-labelled or may contain an affinity purification or attachment moiety.

The kit may further comprise amplification reagents and also other suitably packaged reagents and materials needed for the particular amplification protocol. A preferred kit also includes the components necessary to determine if amplification has occurred. The kit may also include, for example, PCR buffers and enzymes; positive control sequences, reaction control primers; and instructions for amplifying and detecting the specific sequences.

### EXAMPLES

The present technology is further illustrated by the following Examples, which should not be construed as limiting in any way.

### Example 1: Materials and Methods

### Patients and Tissue.

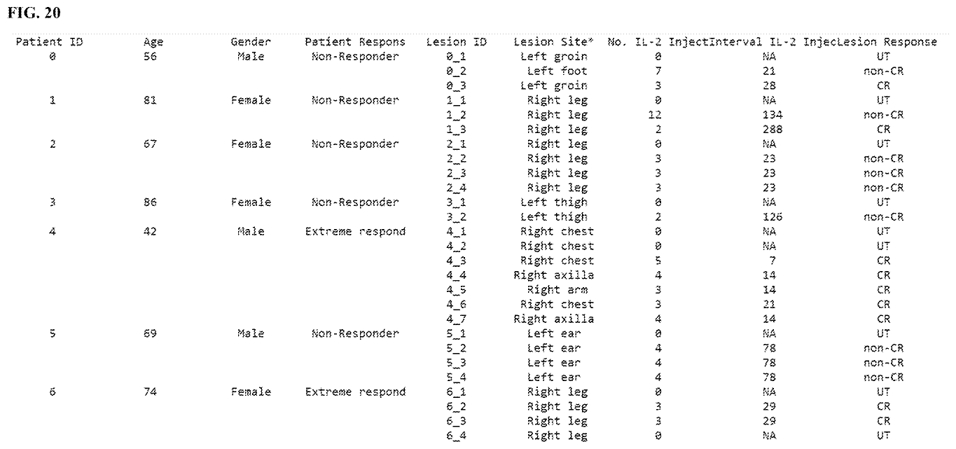

This study includes all patients with in-transit melanoma who presented for intralesional IL-2 therapy at Memorial Sloan Kettering Cancer Center (MSKCC) from 2015-2017 (n=7) and who met the criteria of having pretreatment (untreated) and IL2-injected tissue and subsequent IL2-response data at the level of each injected lesion (FIG. 20). All patients signed statements of informed consent under protocols approved by the MSKCC Institutional Review Board, and the study was conducted in accordance with the Declaration of Helsinki. All lesions were surgically resected and immediately formalin-fixed and paraffin-embedded (FFPE). All biospecimens were obtained as part of routine clinical care with standard FFPE tissue processing in the MSKCC surgical pathology lab (CLIA accredited). FFPE tissue blocks were maintained in the MSKCC Department of Pathology temperature controlled storage units until use. For each specimen, adjacent tissue sections were freshly cut for hematoxylin and eosin (H&E) (1 section, 5-microns), multiplexed immunofluorescence (IF) (Cell Dive, Cytvia, Issaquah, WA) (1 section, 5-microns), NanoString (10 sections, 10-microns), MSK-IMPACT (20 sections, 5-microns), and immunohistochemistry (IHC) for MHC class I (1 section, 4-microns). Cell Dive validation IHCs were completed on 28 additional 4-micron thick tissue sections from lesion 3_2. Multiplexed IF (validation assay) was completed on one 4-micron thick tissue section. H&Es were reviewed by a board-certified Dermatopathologist for classification of treatment response as either complete response (CR) or non-CR based on the presence or absence of tumor cells, respectively.

Our validation cohort includes 19 patients with metastatic melanoma who received intralesional IL-2 or high-dose systemic IL-2 and who met the criteria of having pretreatment (untreated) tissue with no prior therapies and subsequent IL2-response data at the level of each injected lesion for intralesional IL-2 therapy and at the level of the patient for high-dose systemic IL-2 therapy. All patients signed statements of informed consent, and the study was conducted in accordance with the Declaration of Helsinki. All lesions were surgically resected and immediately FFPE using standard FFPE tissue processing. “CR” denotes full regression of the tumor following IL-2 therapy, whereas “non-CR” denotes remaining tumor cells following IL-2 therapy.

### Targeted RNA Sequencing Using NanoString.

NanoString was performed for all lesions in the initial discovery cohort with the exception of lesions 3_2 and 6_2, which were excluded due to low RNA quantity post-extraction. RNA extraction was performed after macrodissection to exclude necrotic and normal skin regions. FFPE sections were deparaffinized using the mineral oil method. Briefly, 800 μL mineral oil (Thermo Fisher catalog #AC415080010) was mixed with the sections and the sample was incubated at 65° C. for 10 minutes. Phases were separated by centrifugation in 360 μL Buffer PKD (DNeasy Blood & Tissue Kit, QIAGEN catalog #69504), and Proteinase K (600 mAU/mL) (DNeasy Blood & Tissue Kit, QIAGEN catalog #69504) was added for digestion. After a three-step incubation (65° C. for 45′, 80° C. for 15′, 65° C. for 30′) and additional centrifugation, the aqueous phase containing RNA was removed and DNase treated (DNeasy Blood & Tissue Kit, QIAGEN catalog #69504). The RNA was then extracted using the RNeasy FFPE Kit (QIAGEN catalog #73504) according to the manufacturer's instructions. A minimum of 100 ng of total RNA per sample was used to measure the expression of 770 immune-related genes and 20 internal reference genes (PanCancer IO 360 gene expression panel) using NanoString Technologies' nCounter platform. Normalization using the internal reference genes was performed using nSolver. Differential expression analysis was run using the DESeq2 Bioconductor package.

### Targeted DNA Sequencing Using MSK-IMPACT.

MSK-IMPACT was performed for all tumor-containing untreated and non-CR lesions in the initial discovery cohort with the exception of patient 6 due to tissue availability. DNA extraction was performed after macrodissection to exclude necrotic and normal skin regions. FFPE tissue was deparaffinized using heat treatment (90° C. for 10′ in 480 μL PBS and 20 μL 10% Tween 20), centrifugation (10,000×g for 15′), and ice chill. Paraffin and supernatant were removed, and the pellet was washed with 1 mL of 100% ethanol followed by an incubation overnight in 400 μl of 1M NaSCN for rehydration and impurity removal. Tissues were subsequently digested with 40 μl of Proteinase K (600 mAU/ml) in 360 μl Buffer ATL (DNeasy Blood & Tissue Kit, QIAGEN catalog #69504) at 55° C. DNA isolation proceeded with the DNeasy Blood & Tissue Kit (QIAGEN catalog #69504) according to the manufacturer's protocol modified by replacing buffer AW2 with 80% ethanol. DNA was eluted in 0.5× Buffer AE heated to 55° C. Next-generation sequencing of patient-matched tumor/normal DNA was performed in the MSKCC Integrated Genomics Operation core facility using MSK-IMPACT 468, which has been previously described (8). A genetically matched normal was used for all cases. The current analysis framework can be found at github.com/mskcc/roslin-variant/wiki/Roslin-Output-v2.5.

### H&E Staining.

H&E staining was performed for all lesions (initial discovery and validation cohorts) using the Ventana Symphony automated H&E stainer with standard clinical protocol. Tissue sections were baked for one hour at 60° C., hydrated, stained with hematoxylin (Leica catalog #3801560, stained with bluing reagent (Leica catalog #3802918), stained with eosin counsterstain (Leica catalog #3801600), rinsed, dehydrated, and coverslipped.

### Multiplexed Immunofluorescence (Cell Dive).

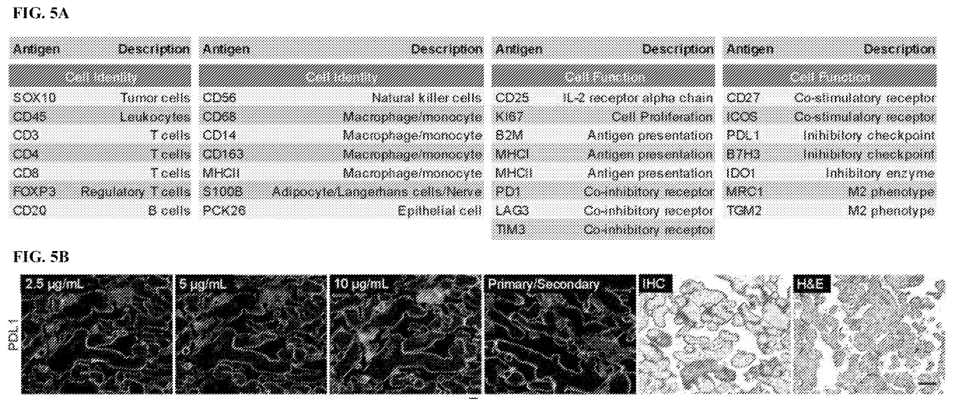

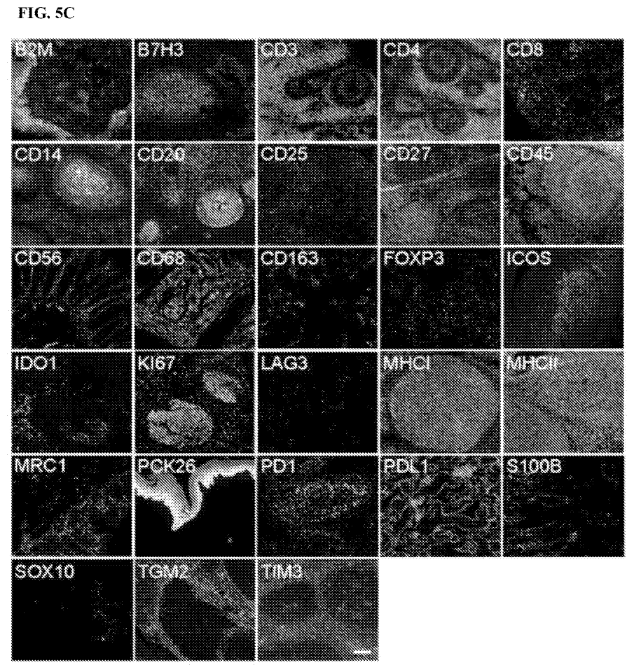

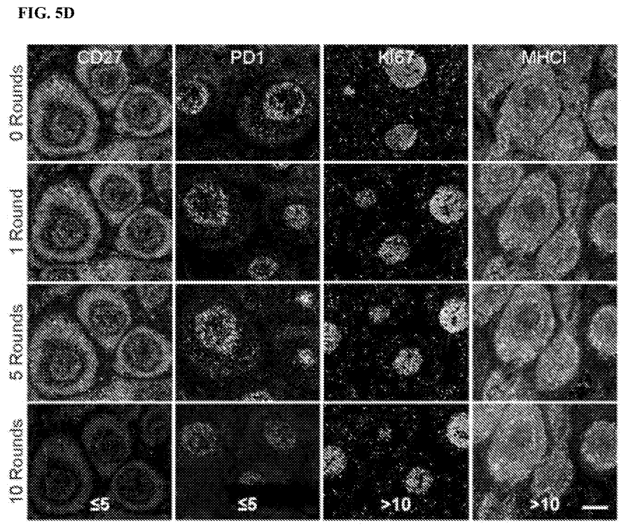

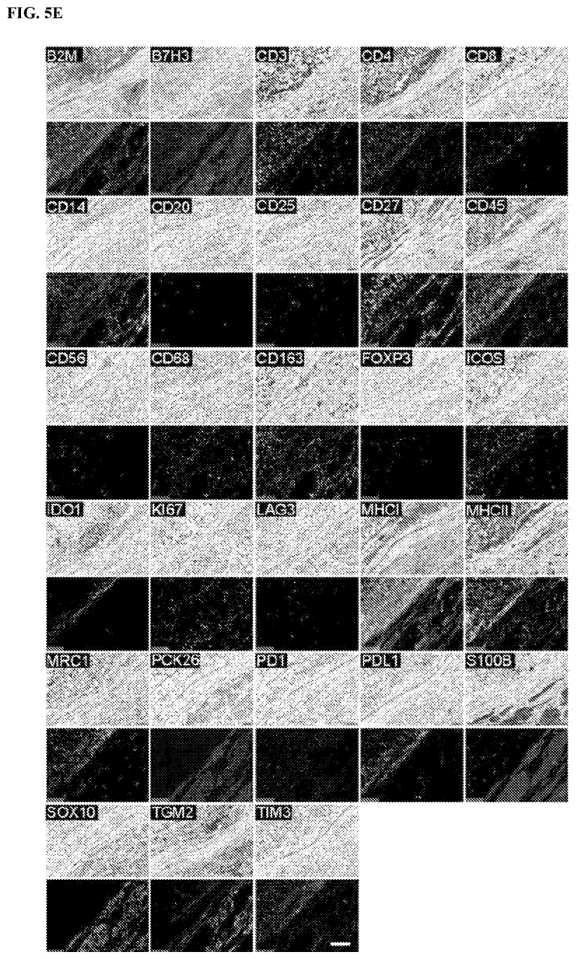

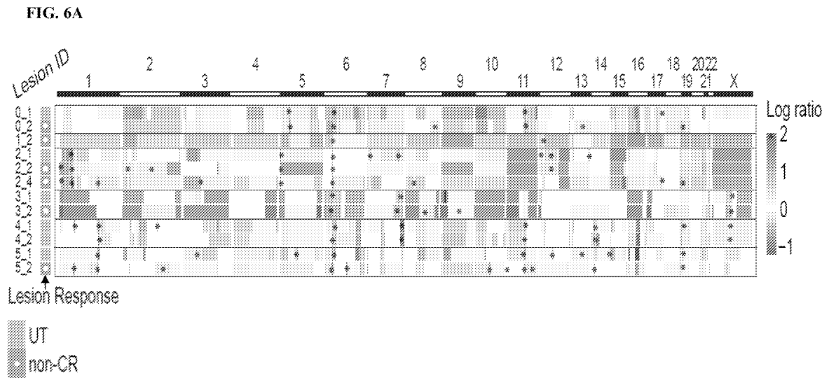

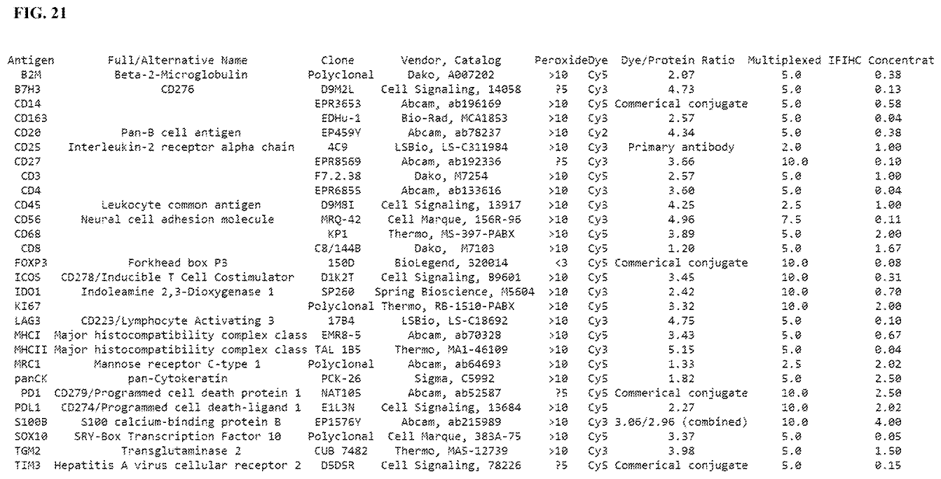

Multiplexed IF (Cell Dive) was performed for all lesions in the initial discovery cohort. Multiple primary antibodies clones were evaluated for each antigen by IHC on normal human multi-tissue controls (tonsil, placenta, skin, colon, kidney, pancreas, testicle, lung, and spleen) (FIG. 5, FIG. 21). The normal human controls were processed with standard FFPE tissue processing in the MSKCC surgical pathology lab (CLIA accredited), and FFPE tissue blocks were maintained in the MSKCC Department of Pathology temperature controlled storage units until use. All patients signed statements of informed consent under protocols approved by the MSKCC Institutional Review Board, and the study was conducted in accordance with the Declaration of Helsinki. The most optimal clone was conjugated to Cy2, Cy3, or Cy5 Bis NHS Ester dyes (GE catalog #PA22000, PA13000, PA25000, respectively) using a previously published protocol (9). Each conjugated primary antibody was evaluated at three different dilutions to the unconjugated antibody. The staining protocol consists of incubating slides for 60 minutes with the conjugated primary antibody and a 3% BSA in PBS diluent at room temperature followed by 3 rounds of 5 minutes washes in 1×PBS. Each epitope was tested for stability to alkaline H₂O₂-based signal inactivation by exposing adjacent sections of a multi-tissue control to 0, 1, 5, and 10 cycles of alkaline H₂O₂ followed by staining with the antibody (9).

The multi-tissue control was included on each slide for quality control. Tissue sections were baked for one hour at 60° C., deparaffinized, hydrated, processed through a 2-step antigen retrieval process (step 1: citrate-based pH 6.0, Vector catalog #H-3300; step 2: EDTA-based pH 8.5, Sigma catalog #T6066-100G/Biorad catalog #161-0729/Sigma catalog #P9416) using a previously published protocol (9), and blocked overnight using normal donkey serum (Jackson ImmunoResearch catalog #017-000-121). The tissue was then stained with DAPI for 15 minutes (Thermo Scientific catalog #D3571) and washed 3 rounds for 5 minutes with 1×PBS. A whole slide image was acquired for field of view (FOV) selection. FOVs were placed on the tumor-stroma interface, tumor center, and regressed tumor. Tissue sections then underwent 16 cycles of background imaging, staining, imaging, and signal inactivation. Images were acquired using the Cytell Cell Imaging System (Cytvia, Issaquah, WA). Image App software was used for image acquisition and registration (using DAPI), and this functionality is fully incorporated into the commercial Cell Dive product (Leica Microsystems). An acquired background image following each cycle of dye inactivation was used to subtract autofluorescence from the subsequent stain round resulting in autofluorescence removed images.

### Multiplexed Immunofluorescence (Validation Assay).

Multiplexed IF (validation assay) was performed for all lesions in the initial discovery cohort. Tissue sections were processed using the protocol described for IHC through antigen retrieval followed by 3 sequential cycles of staining (CD4: 0.7 microgram/mL, CD8: 0.125 microgram/mL, TIM-3:0. 15 microgram/mL) with each round including a 30-minute combined block and primary antibody incubation (Akoya opal antibody diluent/block catalog #ARD1001). The CD4, CD8, and TIM-3 antibodies used can be found in FIG. 21. CD4 and CD8 detection was performed using 10-minute incubation with a goat anti-mouse Poly HRP secondary antibody (Invitrogen catalog #B40961). The HRP-conjugated secondary antibody polymer was detected using fluorescent tyramide signal amplification using Opal dyes 520 (CD4), 570 (CD8), 650 (TIM-3) (Akoya catalog #FP1487001KT, catalog #FP1488001KT, and catalog #FP1496001KT, respectively). The covalent tyramide reaction was followed by heat-induced stripping of the primary/secondary antibody complex using Akoya AR9 buffer (catalog #AR900250ML) and Leica Bond ER2 (90% ER2 and 10% AR9) at 100° C. for 20 minutes preceding the next cycle. After 3 sequential rounds of staining, sections were stained with Hoechst (Invitrogen catalog #33342) and mounted with ProLong Gold antifade reagent mounting medium (Invitrogen catalog #P36930). Whole slide images were acquired using Zeiss AXIO scanner. Indica Labs' HALO Image Analysis software was used for image analysis. Necrotic regions were excluded.

### Immunohistochemistry.

IHC for MHC class I was performed for all lesions (initial discovery and validation cohorts) and for the 28 markers in the multiplexed IF panel (FIG. 21) for lesion 3_2 using an automated staining system (Leica Bond RX) with 3,3′ Diaminobenzidine (DAB) detection (Leica Bond Polymer Refine Detection catalog #DS9800). Tissue sections were baked for 3 hours at 62° C. in vertical slide orientation with subsequent deparaffinization performed on the Leica Bond RX. Antigen retrieval was conducted for 30 minutes using Leica Bond epitope retrieval solution 2 (ER2) (EDTA, pH 9.0) (catalog #AR9640) followed by incubation of the primary antibody at previously optimized concentrations for 30 minutes (a list of the primary antibodies used can be found in FIG. 21) followed by incubation of the secondary antibody (Leica Bond Polymer Refine Detection catalog #DS9800). MHC class I staining was scored for the percentage of membrane-positive tumor cells within the entire tissue section in 5% increments (0% to 100%). Slides were scored blindly. IHC staining for the 28 markers for lesion 3_2 was visually inspected alongside the multiplexed IF (Cell Dive) staining for the 28 markers for lesion 3_2 to ensure accuracy of the multiplexed IF method.

### Multiplexed Immunofluorescence Analysis (Cell Dive).

### Image Analysis:

HALO was used for image visualization and analysis. For each FOV, images for the 28 markers and DAPI were stacked. Markers with technical issues or non-specific staining in either a single FOV or the lesion were excluded. High intensity artifacts were annotated for exclusion. The tumor-stroma interface was manually annotated for each interface FOV using the marker SOX-10, which labels the nuclei of tumor cells. Annotation coordinates were exported for downstream analysis. Nuclear segmentation parameters and thresholds were set for each lesion and optimized using 2 FOVs.

### Cell Loss Computation:

DAPI images (first and last cycles 1 and 32) were processed with intensity normalization and histogram matching. Sum of squared differences was used to generate a pixel level bit mask image highlighting areas of cell loss/drift between images. The bit mask and cell coordinates were used to calculate a loss/drift percentage for each cell.

### Data Processing:

Each cell was assigned a unique ID. Cells in regions with artifacts, in the 20-micron border region of each FOV, and with greater than 10% loss/drift of pixels were removed from analysis. For each interface FOV, we created a quad tree consisting of pixel coordinates of the annotated tumor interface using R-package Search Trees v0.5.2. For each cell, we identified the nearest point on the tumor interface using k-nearest neighbor lookup on the tree and measured the distance from the cell to that point (0.293 microns per pixel conversion). Each cell falling within +/−360-microns of the tumor interface was assigned to a 10-micron interval. Distance in microns between all unique pairs of cells in each FOV was calculated for cell neighborhood analyses. Intensity values for each marker were normalized by dividing the intensities for each marker by the value of the threshold. Intensity values below the threshold (less than 1) were flattened to 1. The log of the intensity values for each marker was divided by the width of the log intensity (97.5-percentile value) distribution across the FOV. Cells were assigned to a cell type using positive and negative combinations of cell identity markers. A method was created to reset problematic thresholds.

t-SNE Analysis:

We performed dimensionality reduction on the full set of marker intensities using the Rtsne package. The normalized/transformed intensities were projected onto 2 dimensions using the 1-SNE method with a perplexity of 250 and 5,000 iterations.

### Statistical Analysis:

We transformed fractions to log odds and used R function wilcox.test( ) with the default two-sided option to compute significance of the differences and effect sizes as log odds ratios (OR). We used the same method log-transformed densities, reporting effect sizes as fold changes. P-values were adjusted for multiple testing with Bonferroni adjustment.

### Statistical Integration of Overall and Intra-Patient Analyses:

From filtered cell fractions for overall CR versus untreated and overall non-CR versus untreated comparisons, we filtered cell fractions with at least one significant overall OR (p-adjusted<0.05). Harmonic mean p-value (HMP) was calculated for each intra-patient comparison and adjusted using Bonferroni test. The union of untreated lesion FOVs was used for patients with 2 untreated lesions (#4, #6).

### Tumor MHC Class I Neighborhoods:

Immune cells were grouped by being in MHC-I-low, MHC-I-mixed, MHC-I-high, tumor-free, and isolated neighborhoods (30-micron radius) based on neighborhoods having a maximum of 25% MHC-I′ tumor cells, between 25-75% MHC-I′ tumor cells, a minimum of 75% MHC-I′ tumor cells, no tumor cells, or no neighbors, respectively.

### Identification of B-Cell Aggregates.

Clusters of B cells were identified using the set of B-cell pairs with less than 30 microns between them. Starting with one pair as a cluster, all B-cell neighbors of both cells in the pair were added to the cluster, followed by neighbors of neighbors. Clusters of 20 B cells or more were labeled B-cell aggregates.

### CD8⁺ T-Cell Neighborhoods.

Immune cells were grouped by being in CD8⁺ T-cell triple-negative (TN), single-positive (SP), double-positive (DP), or triple-positive (TP) neighborhoods using PD-1, LAG-3, and TIM-3 based on neighborhoods having exclusively one degree of CD8⁺ T-cell exhaustion (TN, SP, DP, or TP). All other immune cells were excluded from the analysis. Log OR of each cell fraction in groups SP, DP, TP versus TN was computed. Cell fractions were tested for monotonicity as exhaustion progresses using R package Kendall.

### Data and Materials Availability.

All data supporting findings of this study are available online at doi.org/10.5281/zenodo. All code and detailed computational methods for multiplexed IF and NanoString analysis are available at github.com/mskcc/Halo_Melanoma_IL2.

### Example 2: Membranous MHC Class I is a Biomarker for IL-2 Therapy

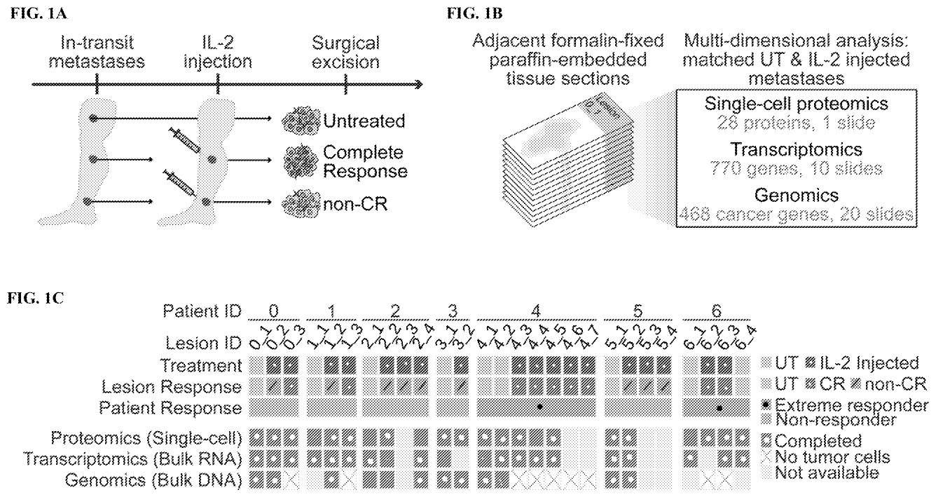

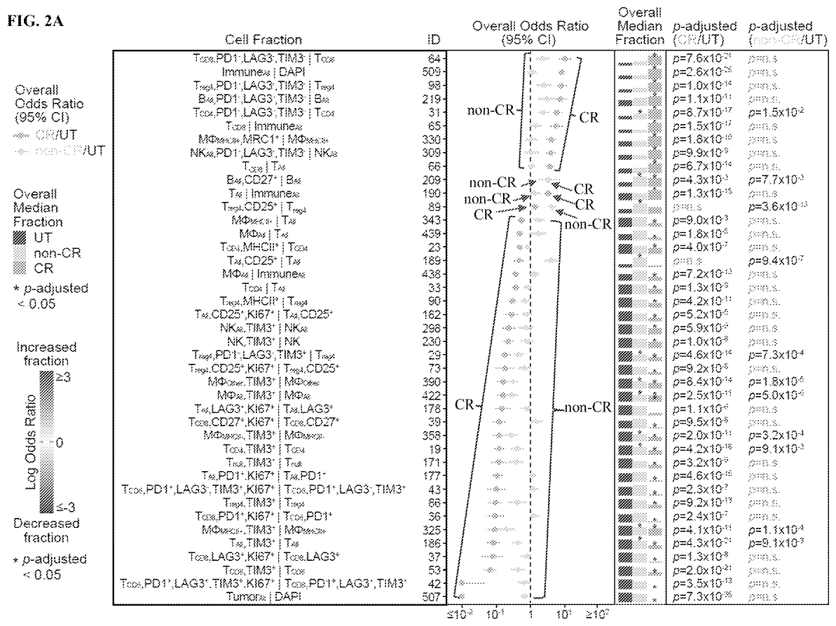

We identified seven patients with multiple contemporaneous in-transit melanoma metastases, of which at least one metastasis was surgically removed without prior IL-2 injection (termed untreated) and at least one metastasis had received IL-2 injections prior to resection (FIG. 1A). This cohort of matched untreated and IL2-injected lesions provided an opportunity to investigate pretreatment molecular and cellular makeup as well as IL2-associated changes within the tumor that are associated with tumor response to therapy. For each lesion, we assessed the co-expression of 28 proteins at single-cell resolution (using multiplexed immunofluorescence, multiplexed IF (9, 10), the abundance of 770 immune response-related transcripts (bulk RNA), and the presence or absence of genetic alterations in 468 cancer genes (bulk DNA) in immediately adjacent FFPE tissue sections (FIG. 1B).

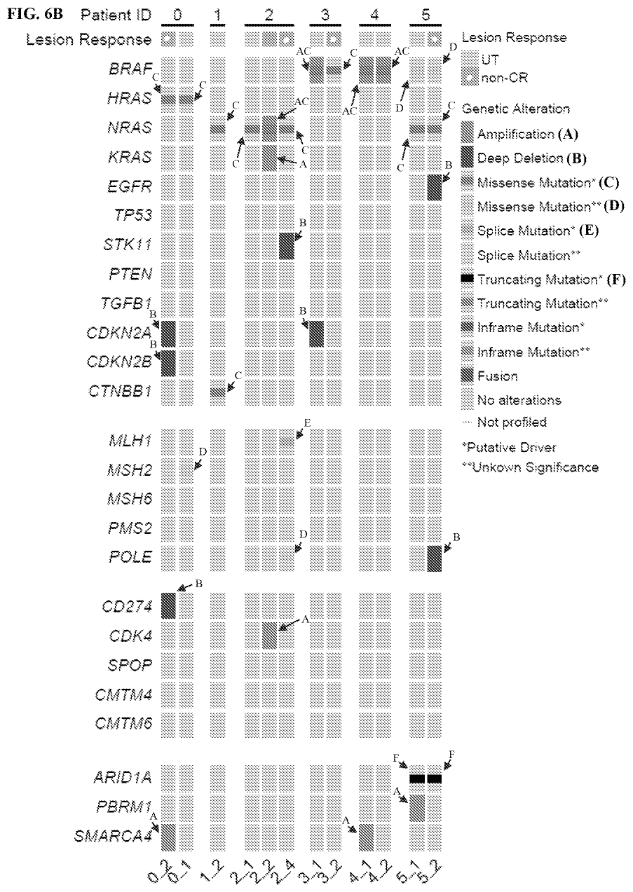

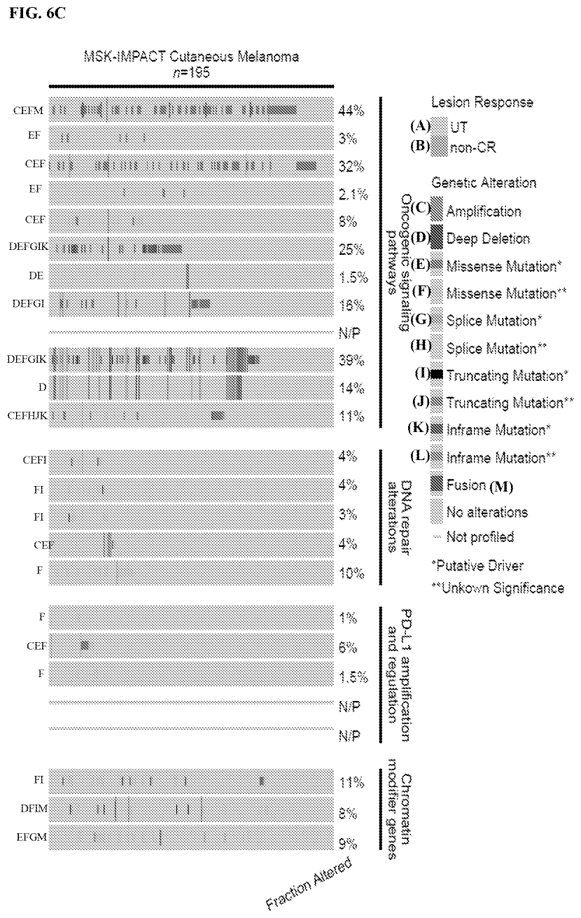

In total, 9/18 (50%) IL2-injected lesions responded completely to intralesional IL-2 (termed CR), defined by the absence of tumor cells on histopathological review. The other 9/18 IL IL2-injected lesions contained abundant residual tumor cells (termed non-CR; FIG. 1C, FIG. 20). Two patients (termed extreme responders) experienced complete regression of all IL2-injected lesions, whereas all other patients (termed non-/mixed responders) had at least one IL2-resistant lesion. Metastases from a given patient harbored similar mutations and copy number profiles (FIGS. 6A-6B), supporting their origin from the same primary tumor clone. When compared to a larger cohort of cutaneous melanomas that were sequenced using MSK-IMPACT (8), our cohort showed a similar distribution of genetic alterations (FIGS. 6B-6C).

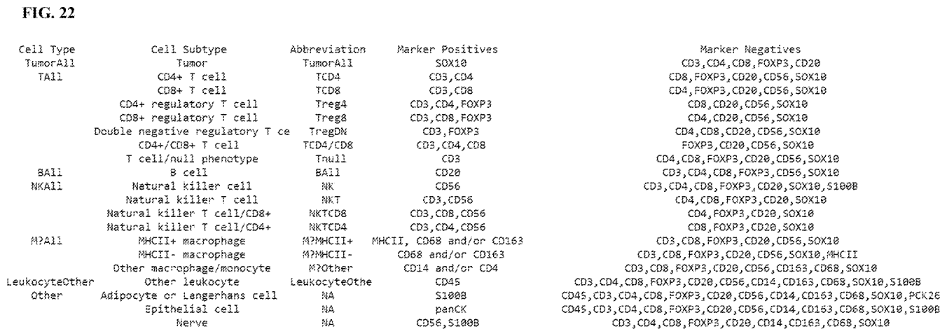

Our selection of markers for single-cell proteomic analysis included markers for both cell identity (e.g., tumor cell) and cell function (e.g., antigen presentation) (FIG. 5A, FIG. 21). For each marker, we evaluated specificity and sensitivity of the fluorescent dye-conjugated antibody through staining of normal human tissues (FIGS. 5B-5C), epitope stability to H₂O₂-based signal inactivation through repeated dye inactivation cycles (FIG. 5D), and multiplexed IF staining specificity through staining of adjacent tumor sections with standard IHC (FIG. 5E). Using combinations of positivity and negativity for cell identity markers, we defined 16 immune cell types, tumor cells, adipocyte/Langerhans cells, epithelial cells, and nerve cells (FIG. 22).

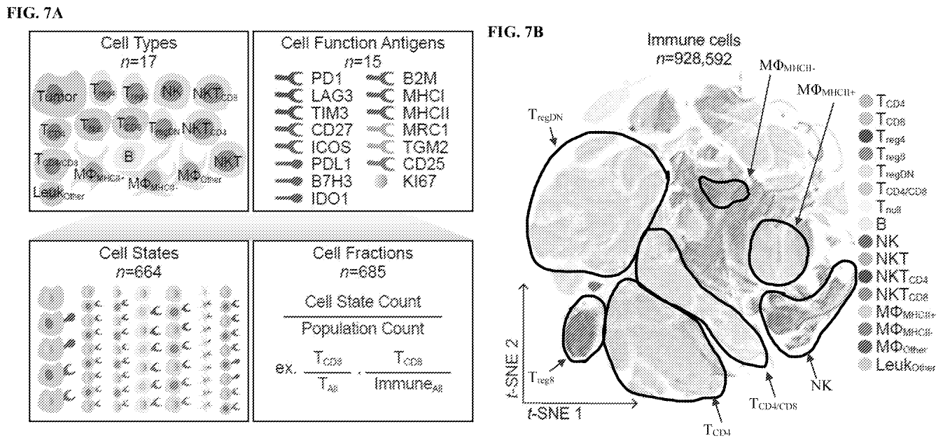

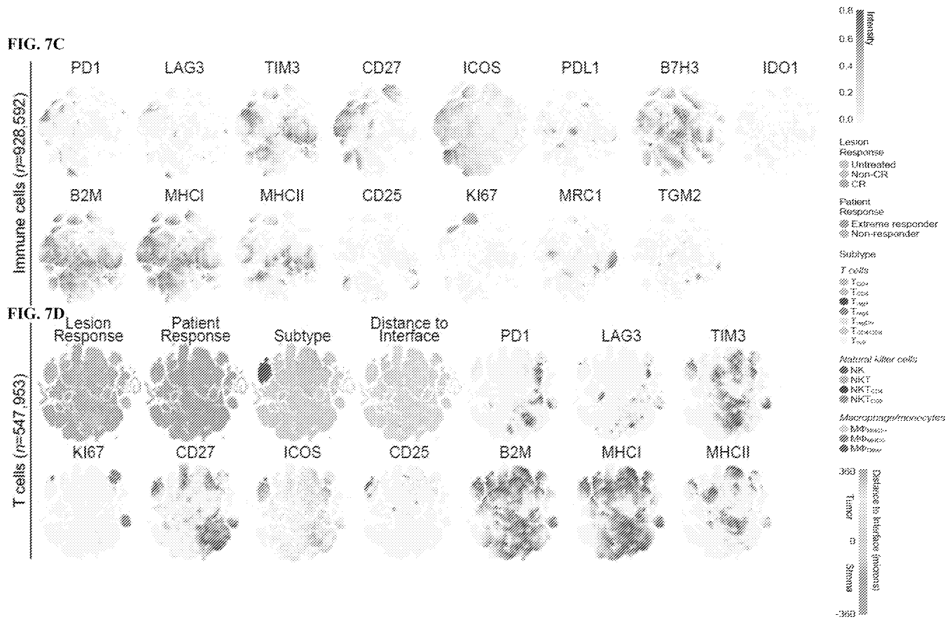

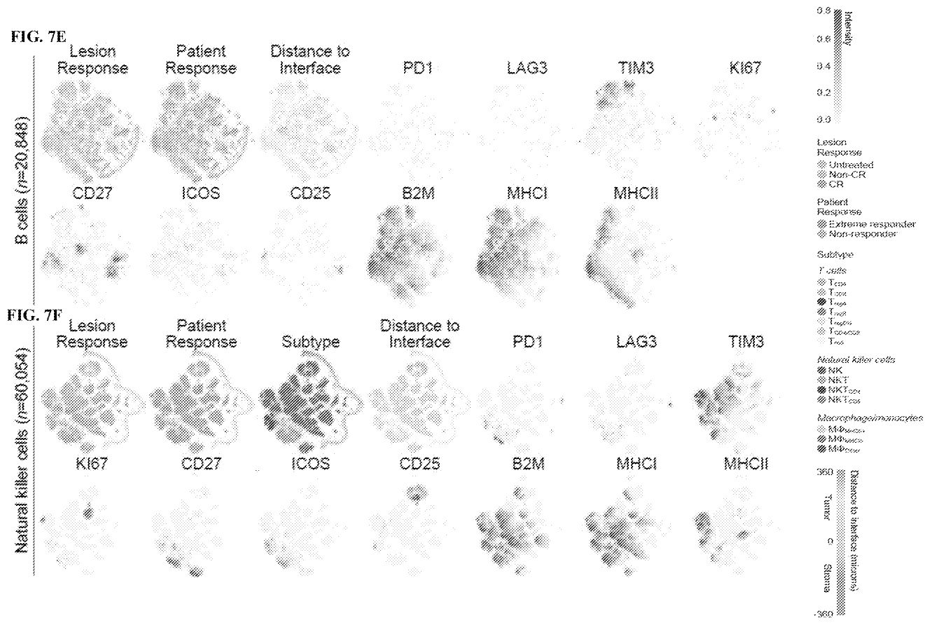

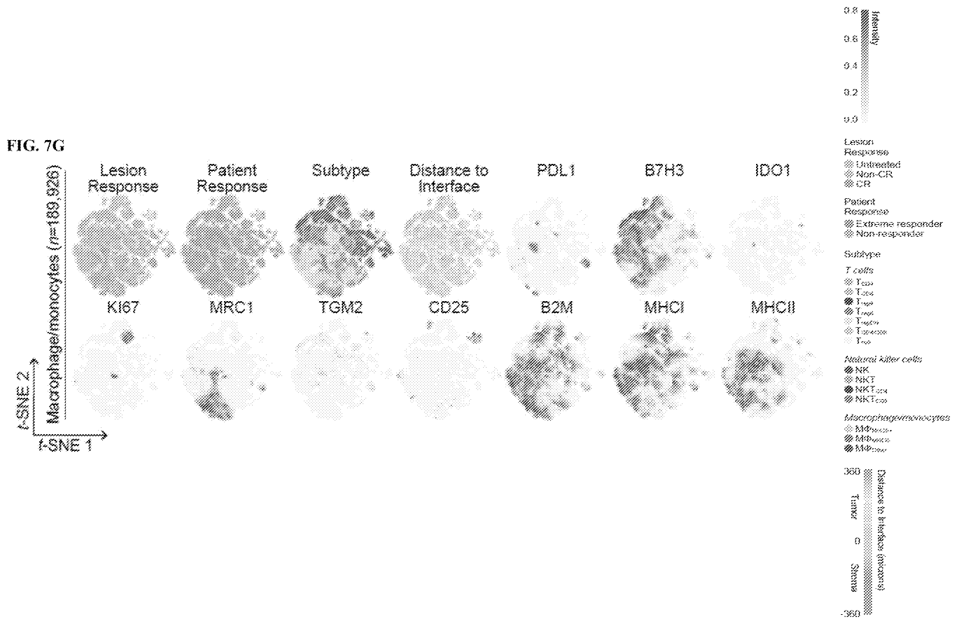

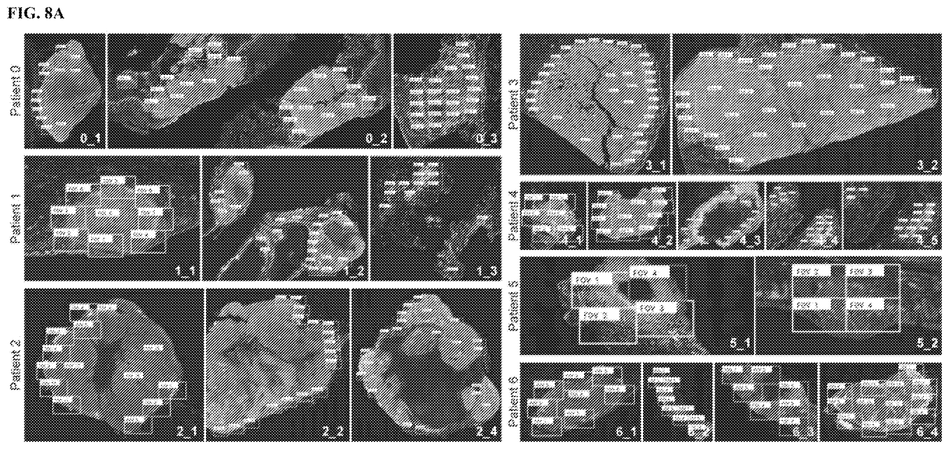

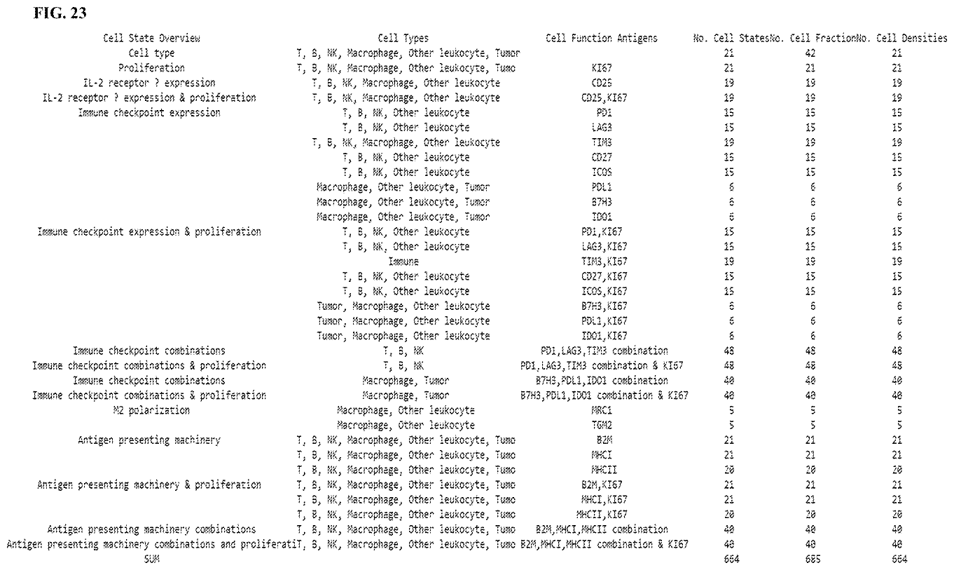

To identify functionally distinct subpopulations of tumor and immune cells, we annotated each cell type with combinations of 15 cell-function markers, ultimately resulting in 664 distinct “cell states”. For comparisons between samples, the relative abundance of these subpopulations was expressed as cell fractions (e.g., fraction of Ki67⁺ CD8⁺ T cells over all CD8⁺ T cells) and cell densities (e.g., Ki67⁺ CD8⁺ T cells per millimeter squared), ultimately amounting to 685 distinct cell fractions and 664 distinct cell densities (FIG. 7, FIG. 23).

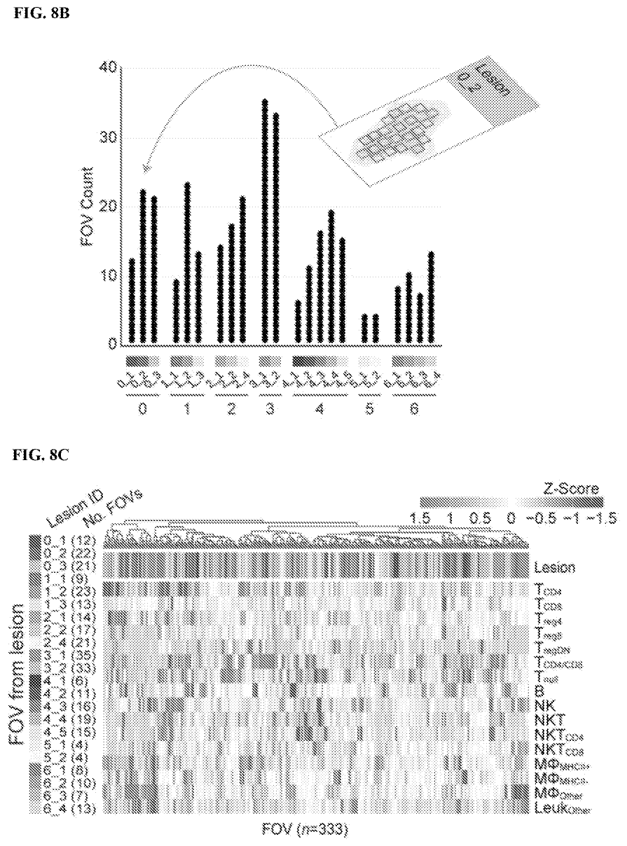

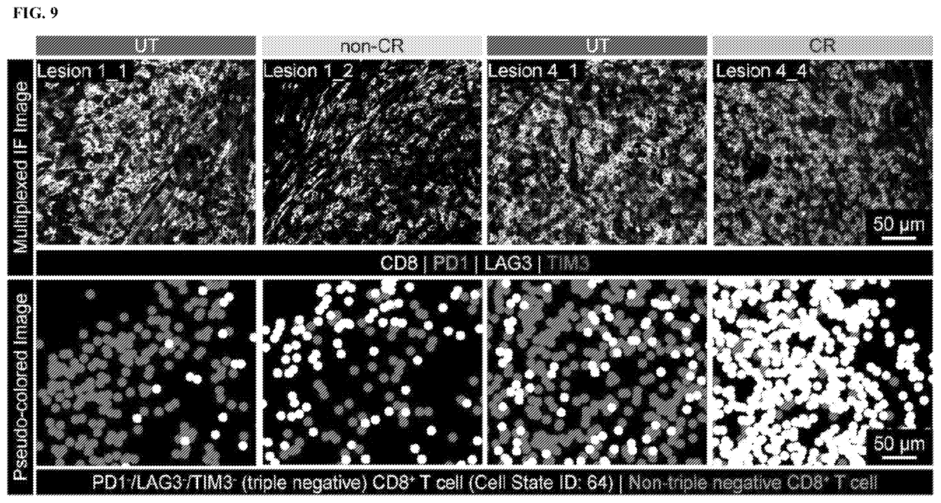

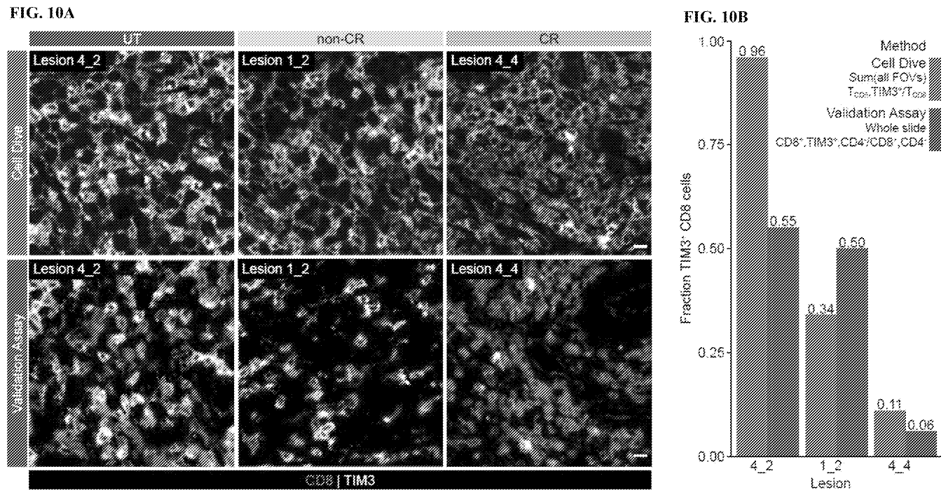

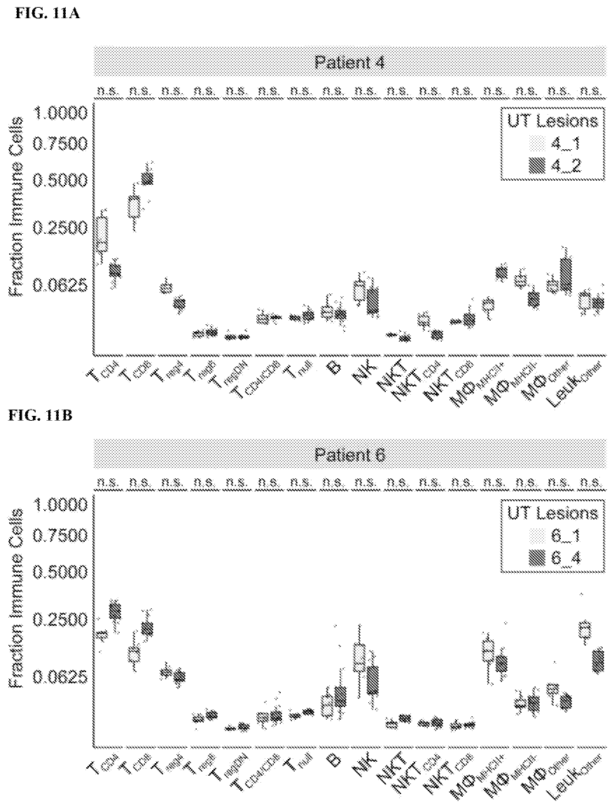

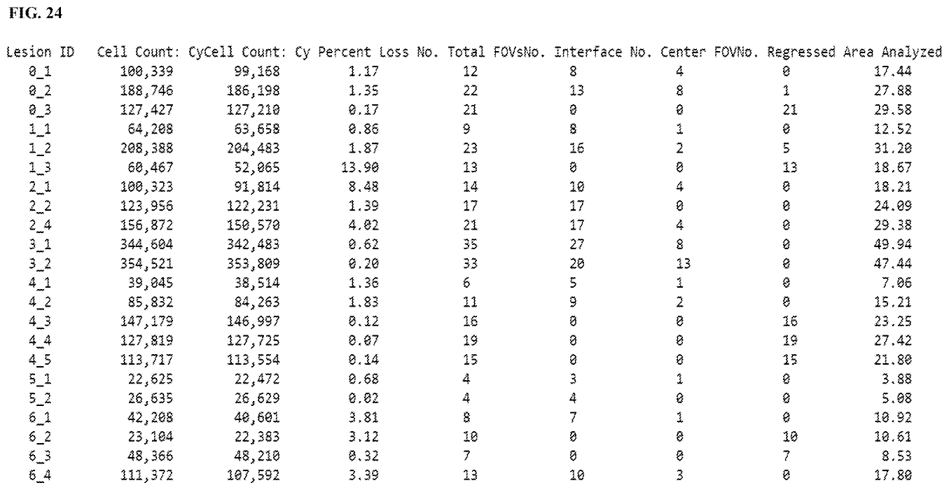

Following cell segmentation, marker thresholding, and removal of cells displaced during any staining cycle, we identified a total of 2,572,629 cells within 333 high-dimensional FOVs from 22 excised lesions in total (untreated, CR, and non-CR) of which 928,592 were immune cells (FIG. 7B, FIG. 24). Examination of multiple FOVs from each lesion allowed us to survey a much broader tumor area than typically examined using tissue microarrays (FIGS. 8A-8B, FIG. 24). We observed considerable heterogeneity in the composition of immune-cell infiltrates within FOVs from the same lesion (FIG. 8C). At the single-cell level, the expression of some markers appeared mostly restricted to specific immune-cell types (e.g., expression of the IL-2 receptor alpha chain CD25 in CD4 regulatory T cells), whereas other markers (e.g., Ki67) were expressed in all immune-cell types (FIG. 7C). Within cell types, certain cell-function markers were more broadly expressed than others (e.g., expression of TIM-3 and CD27 in T cells and B cells) (FIGS. 7D-7G).

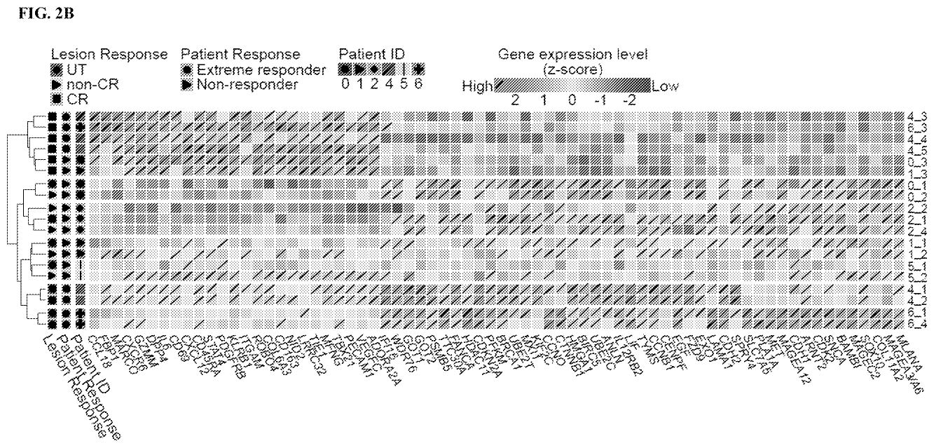

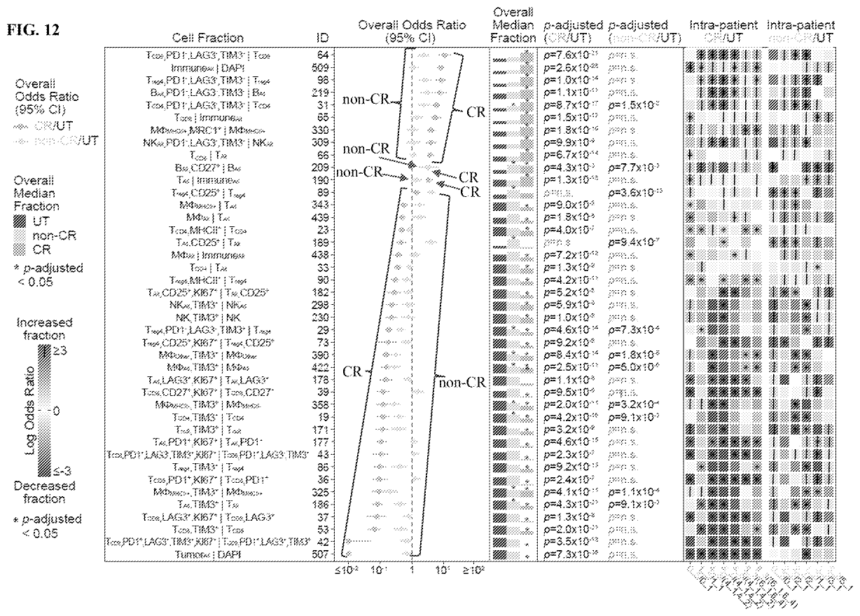

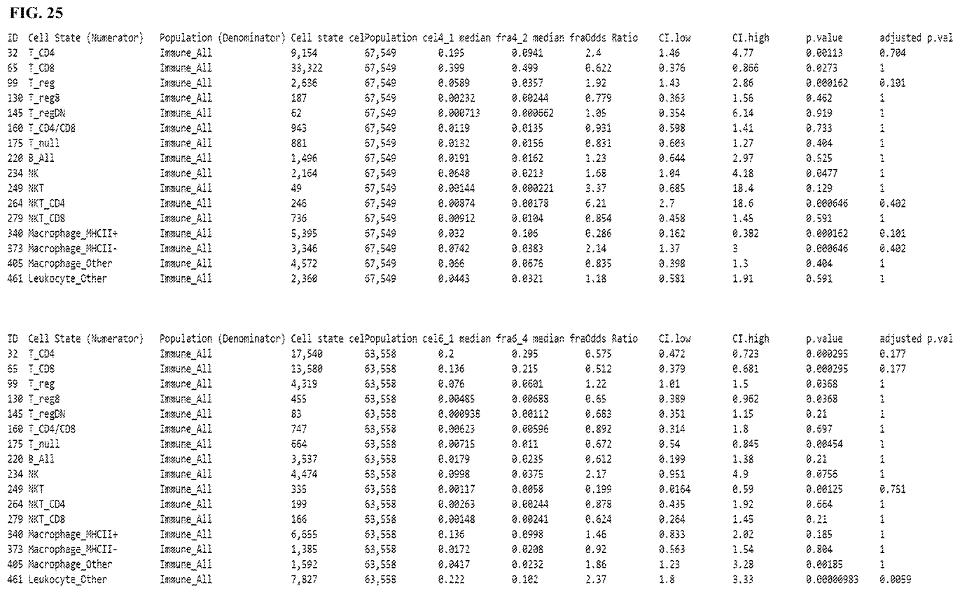

To gain insight into the molecular processes that are associated with a complete lesion response to intralesional IL-2, we determined the frequency of each cell state in untreated and IL2-injected lesions (FIG. 2A). Compared to untreated lesions, CR lesions showed increased fractions of CD8⁺ T cells over all T cell subtypes and all immune cells, a well-documented effect of IL-2 (6, 7). CR lesions also showed increased fractions of PD-1⁻ LAG-3⁻TIM-3⁻CD8⁺ T cells, CD4⁺ (helper) T cells, CD4⁺ regulatory T cells, B cells, and natural killer (NK) cells (FIG. 2A, FIG. 9). CR lesions also showed the lowest fractions of TIM-3⁺ cells (across most immune-cell types), a finding that we validated using an independent multiplexed IF platform (FIG. 2A, FIGS. 10A-10B). This is reminiscent of the reported association between TIM-3 and treatment resistance in other immuno-oncology contexts (11, 12). Non-CR lesions, on the other hand, showed an admixture of immune and tumor cells and no statistically significant change in CD8⁺ T-cell infiltration compared to untreated lesions (FIG. 2A). Interestingly, non-CR lesions did harbor increased numbers of CD25⁺ CD4⁺ regulatory T cells, suggesting that failure to upregulate the IL-2 receptor alpha (CD25), a well-documented effect of IL-2 (13), is not the cause of IL-2 resistance in these lesions. We observed no differences in immune-cell populations between multiple untreated lesions from the same patient (FIGS. 11A-11B, FIG. 25). Overall, there was considerable heterogeneity in the IL-2 response between patients (FIG. 12). For example, non-CR lesions from two patients showed significantly increased fractions of PD-1⁻LAG-3⁻TIM-3⁻ CD8⁺ T cells. However, at the level of the cohort, this trend did not reach statistical significance.

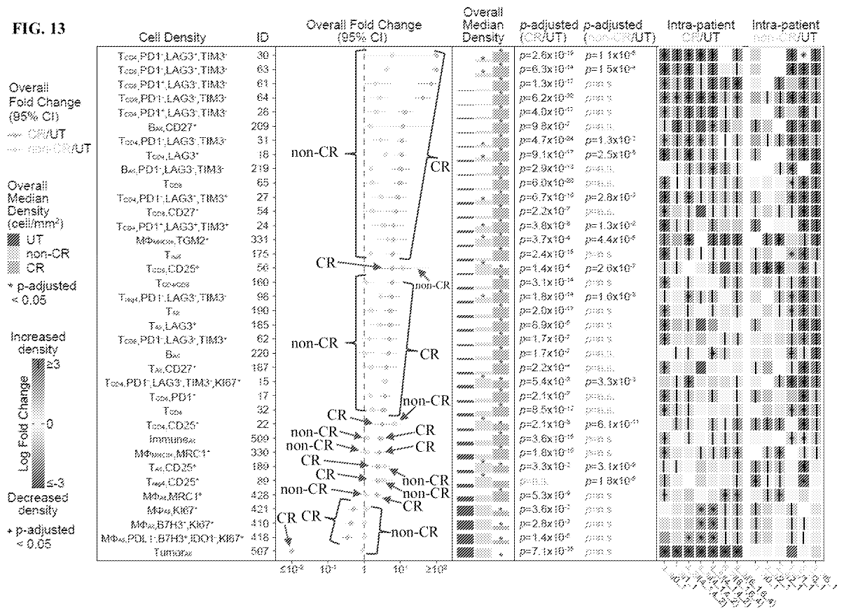

We also analyzed the density of distinct cell states in CR, non-CR, and untreated lesions and again observed increased densities of CD8⁺ T cells and PD-1⁻LAG-3⁻TIM-3⁻ CD8⁺ T cells, CD4⁺ (helper) T cells, CD4⁺ regulatory T cells, and B cells in CR lesions and increased densities of CD25⁺ CD4⁺ regulatory T cells in non-CR lesions, both compared to untreated lesions (FIG. 13). We also observed an increase in the density of B cells and a decrease in the density of proliferating (Ki67⁺) macrophages in CR lesions compared to untreated lesions (FIG. 13).

Our examination of immune-cell states at the single-cell level suggested that effective antitumor immunity consequent to IL-2 injection was characterized by the presence of non-proliferating T cells with a non-exhausted phenotype (PD-1⁻LAG-3⁻TIM-3⁻). At the level of the transcriptome, we identified 70 genes that were differentially expressed in CR lesions compared to untreated lesions (FIG. 2B). This “IL2-response signature” included the upregulation of 25 genes associated with T-cell activation (e.g., ADORA2A, CD69, DPP4, GZMM, STAT4, TBX21) and immune-cell localization to tumors (e.g., CCL18, MARCO, CXCR6, GZMM, DPP4, CD69). Hierarchical clustering of the differentially expressed genes grouped untreated and non-CR lesions from the same patient, suggesting IL-2 injection in the non-CR lesions failed to cause transcriptional reprogramming towards antitumor immunity. Interestingly, the clustering also grouped the 4 untreated lesions from the two patients who experienced complete regression of all injected lesions (extreme responders), indicating the presence of a transcriptional state that favors IL-2 response.

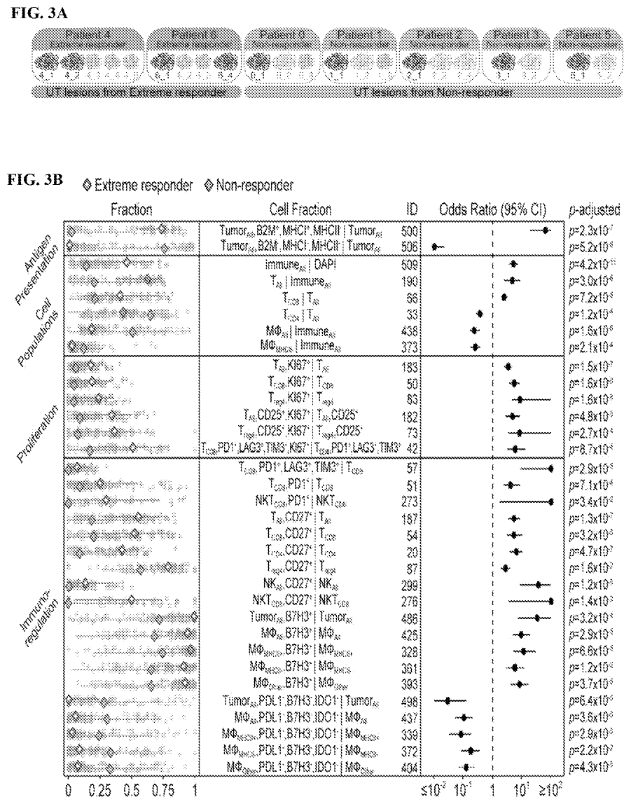

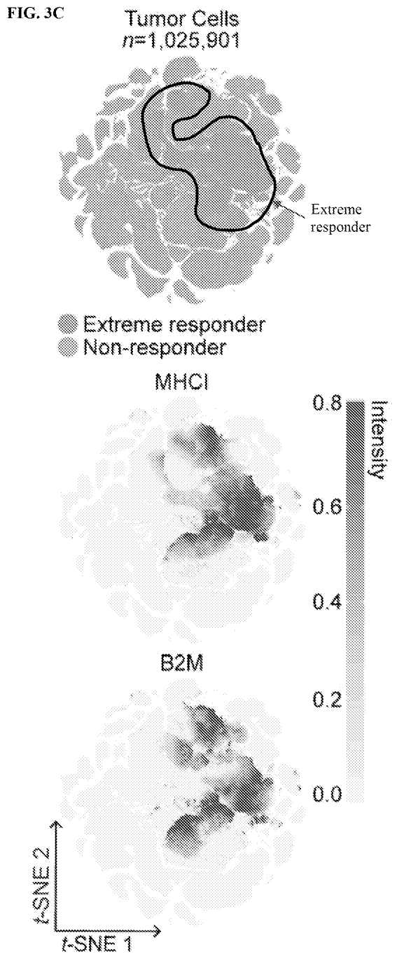

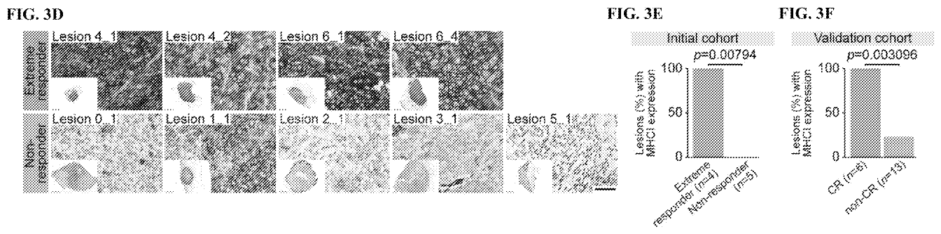

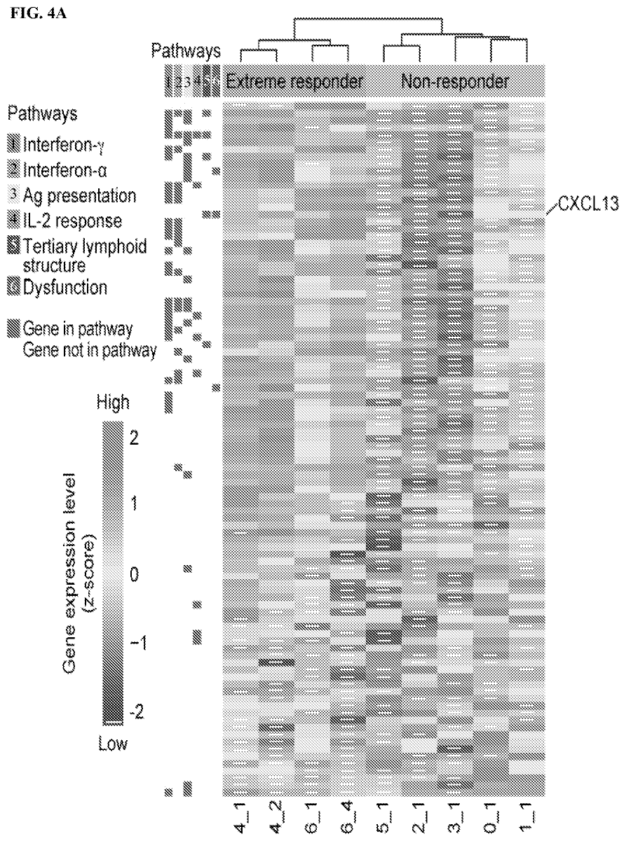

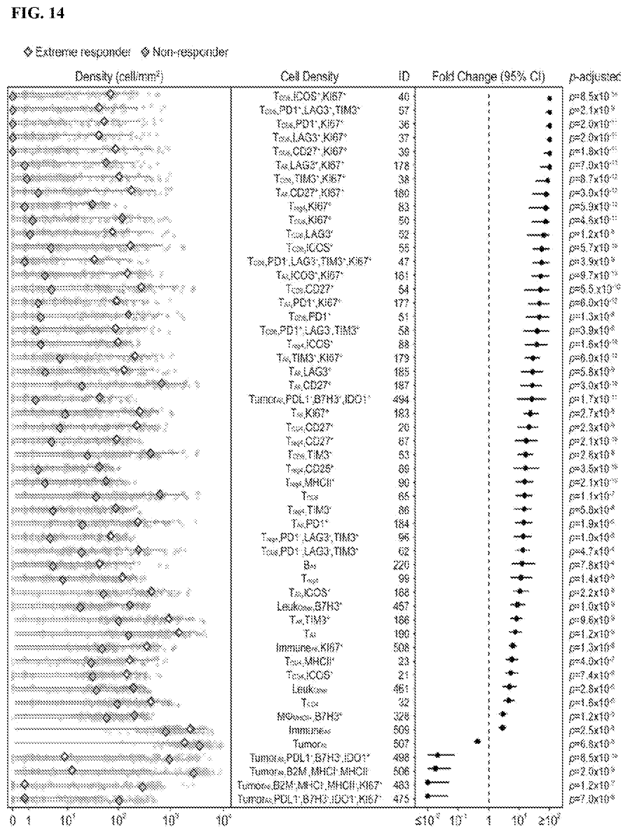

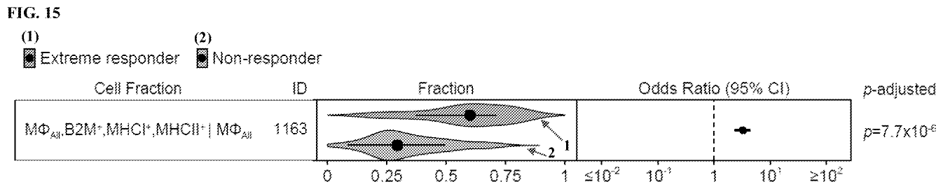

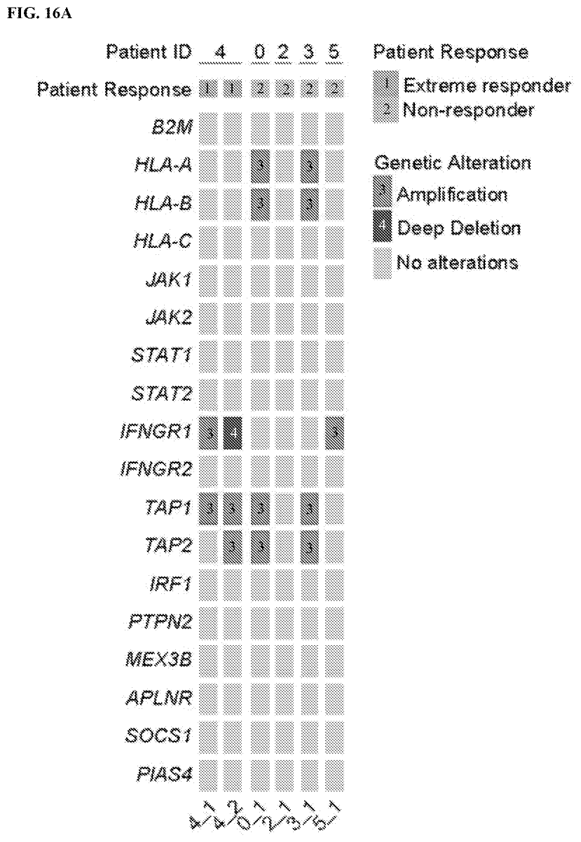

One of the key goals of our analysis was to identify molecular or cellular changes in untreated lesions that might predict a complete lesion response to subsequent IL-2 therapy. We therefore compared untreated lesions from “extreme responders” to the untreated lesions from patients for whom none or only some of the lesions had responded to IL-2 (non-/mixed responders) (FIG. 3A). Untreated lesions from extreme responders harbored a higher fraction of CD8⁺ T cells and a lower fraction of CD4⁺ T cells and MHC-II-macrophages (FIG. 3B). Untreated lesions from extreme responders also had higher fractions of proliferating T cell populations, CD27⁺ “activated” T- and NK-cell populations, B7-H3⁺ macrophage populations and tumor cells, and PD-L1⁻B7-H3⁻IDO-1⁻ macrophage populations and tumor cells. Untreated lesions from extreme responders also harbored a higher fraction of PD-1⁺LAG-3⁺TIM-3⁺ (exhausted) CD8⁺ T cells and both exhausted and proliferating CD8⁺ T cells, suggesting they were tumor reactive. We also observed a higher density of B cells in untreated lesions from extreme responders (FIG. 14).

One of the most consistent differences between untreated lesions from extreme responders compared to non-/mixed responders was the higher fraction of B2M⁺MHC-I⁺MHC-II tumor cells (ID: 500) and lower fraction and lower density of B2M⁻MHC-I⁻ MHC-II tumor cells (ID: 506) (FIG. 3B). In fact, there was near complete overlap between the lack of MHC-I and B2M expression on tumor cells and subsequent IL-2 response failure at the single-cell level (FIG. 3C). We confirmed this finding by IHC staining for MHC-I (FIG. 3D). All untreated lesions from extreme responders exhibited membranous MHC-I positivity in at least 75% of tumor cells, whereas all untreated lesions from non-/mixed responders exhibited membranous MHC-I positivity (if any) in less than 75% of tumor cells (FIG. 3E). We also found higher fractions of B2M⁺MHC-I⁺MHC-II⁺ macrophage/monocytes in untreated lesions from extreme responders, suggesting MHC-I expression in both tumor cells and macrophages is associated with IL-2 response (FIG. 15).

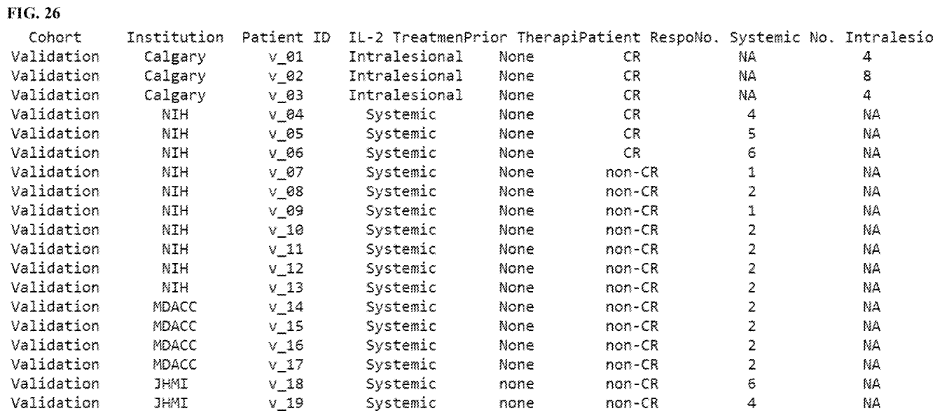

To confirm the association between tumor MHC-I expression and complete response to IL-2, we performed IHC staining for MHC-I in tumor biopsies from 19 previously untreated patients with metastatic melanoma who subsequently received IL-2 therapy (Table 1, FIG. 26).

TABLE 1
Patient characteristics.

| | | | | | | IL-2 | Patient |
|---|---|---|---|---|---|---|---|
| Cohort | Institution | Patient ID | Age | Gender | Lesion Site | Treatment | Response |
| Initial | MSKCC | 4 | 42 | M | Skin | Intralesional | CR |
| Initial | MSKCC | 6 | 74 | F | Skin | Intralesional | CR |
| Initial | MSKCC | 0 | 56 | M | Soft tissue | Intralesional | non-CR |
| Initial | MSKCC | 1 | 81 | F | Skin | Intralesional | non-CR |
| Initial | MSKCC | 2 | 67 | F | Soft tissue | Intralesional | non-CR |
| Initial | MSKCC | 3 | 86 | F | Skin | Intralesional | non-CR |
| Initial | MSKCC | 5 | 69 | M | Skin | Intralesional | non-CR |
| Validation | Calgary | v_01 | 69 | F | Skin | Intralesional | CR |
| Validation | Calgary | v_02 | 86 | F | Skin | Intralesional | CR |
| Validation | Calgary | v_03 | 93 | M | Skin | Intralesional | CR |
| Validation | NIH | v_04 | 52 | M | Soft tissue | Systemic | CR |
| Validation | NIH | v_05 | 43 | M | Skin | Systemic | CR |
| Validation | NIH | v_06 | 45 | F | Lymph node | Systemic | CR |
| Validation | NIH | v_07 | 54 | M | Skin | Systemic | non-CR |
| Validation | NIH | v_08 | 63 | F | Skin | Systemic | non-CR |
| Validation | NIF | v_09 | 52 | M | Soft tissue | Systemic | non-CR |
| Validation | NIH | v_10 | 27 | M | Lymph node | Systemic | non-CR |
| Validation | NIH | v_11 | 48 | M | Soft tissue | Systemic | non-CR |
| Validation | NIH | v_12 | 48 | M | Lung | Systemic | non-CR |
| Validation | NIH | v_13 | 37 | M | Lymph node | Systemic | non-CR |
| Validation | MDACC | v_14 | 62 | M | Lung | Systemic | non-CR |
| Validation | MDACC | v_15 | 76 | F | Skin | Systemic | non-CR |
| Validation | MDACC | v_16 | 65 | M | Lymph node | Systemic | non-CR |
| Validation | MDACC | v_17 | 54 | M | Lymph node | Systemic | non-CR |
| Validation | JHMI | v_18 | 53 | F | Skin | Systemic | non-CR |
| Validation | JHMI | v_19 | 59 | M | Skin | Systemic | non-CR |

Clinical information for the metastatic melanoma patients in the initial cohort (n = 7) and in the validation cohort (n = 19) treated with either intralesional interleukin-2 (IL-2) or high-dose systemic IL-2.
Patient response was classified as complete responder (CR) (termed “extreme responder” in the initial cohort) or non-complete responder (non-CR) (termed “non-/mixed responder” in the initial cohort).
Abbreviations: MSKCC, Memorial Sloan Kettering Cancer Center; Calgary, University of Calgary; NIH, National Institutes of Health; MDACC, MD Anderson Cancer Center; JHMI, Johns Hopkins Medical Institute.

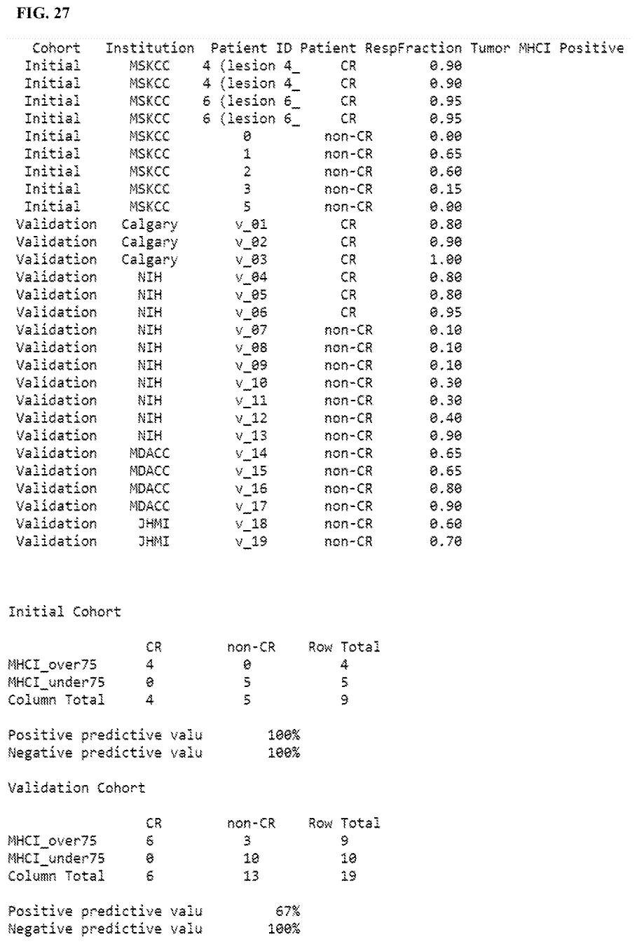

We again found that all patients who had a CR exhibited membranous MHC-I positivity in the vast majority (at least 75%) of tumor cells (FIG. 3F). Conversely, lack of membranous tumor MHC-I expression was strongly associated with no or incomplete tumor response (non-CR) (FIG. 27).

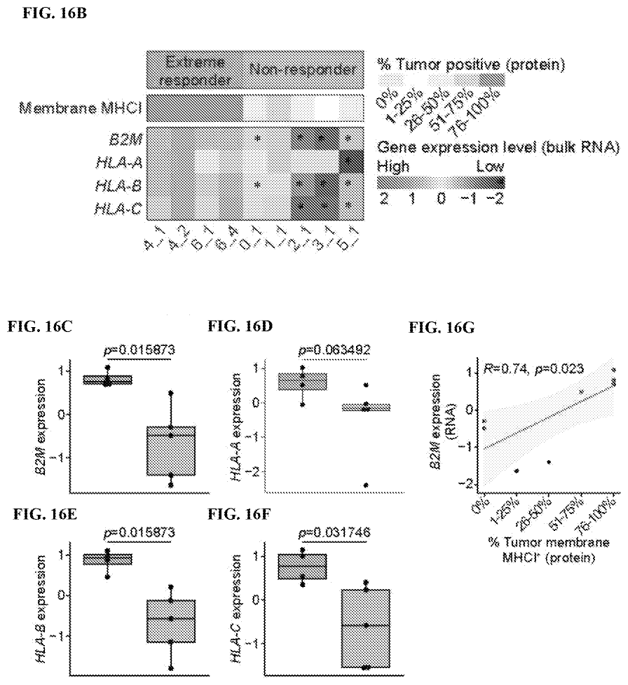

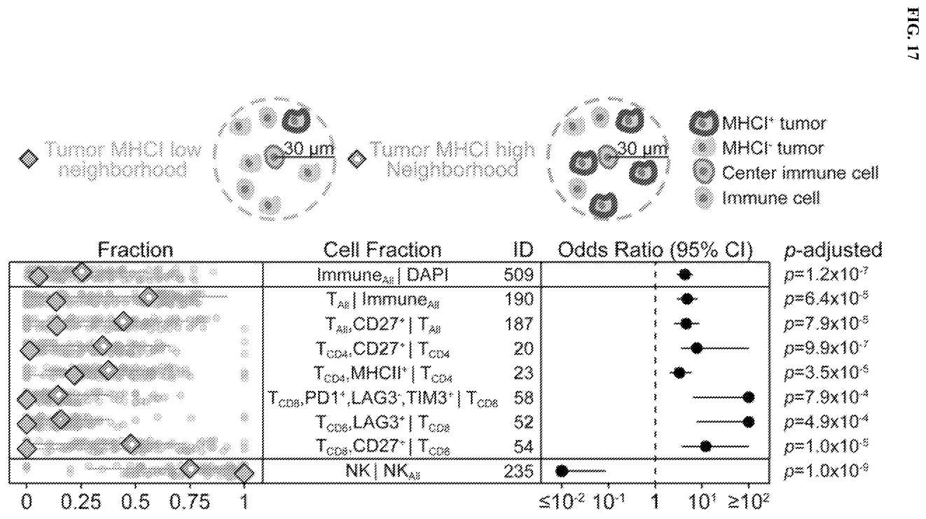

Lack of membranous tumor MHC-I expression was not associated with mutations in B2M or other antigen presentation-pathway related genes (FIG. 16A). Untreated lesions from extreme responders showed significantly increased bulk RNA levels of B2M, HLAB, and HLAC (FIGS. 16B-16F), consistent with our findings at the protein level. Loss of B2M RNA expression in bulk tumor was positively correlated with loss of MHC-I protein expression on the tumor cell membrane (FIG. 16G).

Our data indicated that expression of MHC-I protein on the tumor cell membrane, which is required for antigen presentation, was associated with a higher fraction of exhausted and proliferating CD8⁺ T cells. To explore this relationship in greater detail, we compared the functional state of immune cells in the immediate “neighborhood” (i.e., within 30 μm) of MHC-I-high and MHC-I-low tumor cells (FIG. 17). MHC-I−-high neighborhoods harbored higher fractions of T cells and T-cell populations positive for CD27, PD-1, LAG-3, TIM-3, and MHC-II, while MHC-I-low neighborhoods harbored higher fractions of NK cells, consistent with the known inhibitory effect of MHC-I on NK cells (14) and a dominant innate immune response in MHC-I-low lesions.

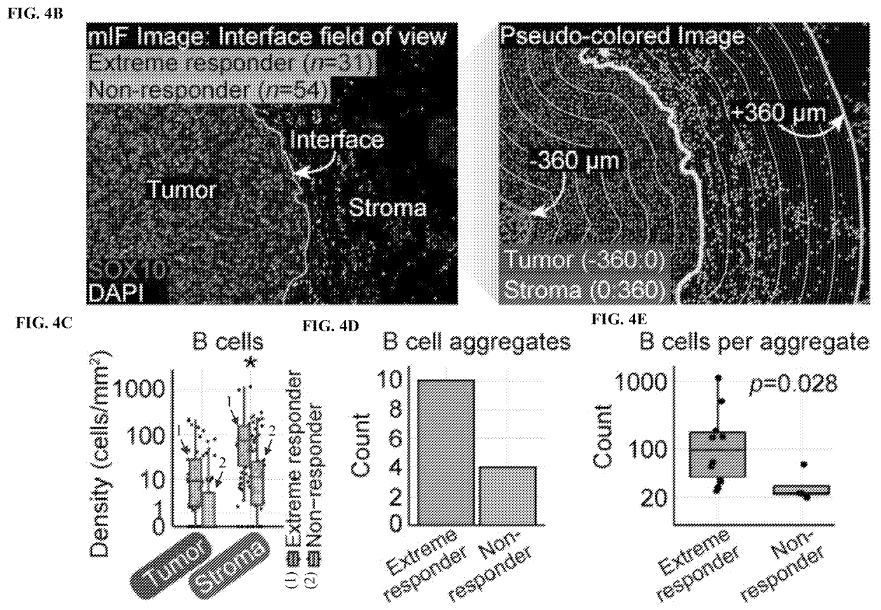

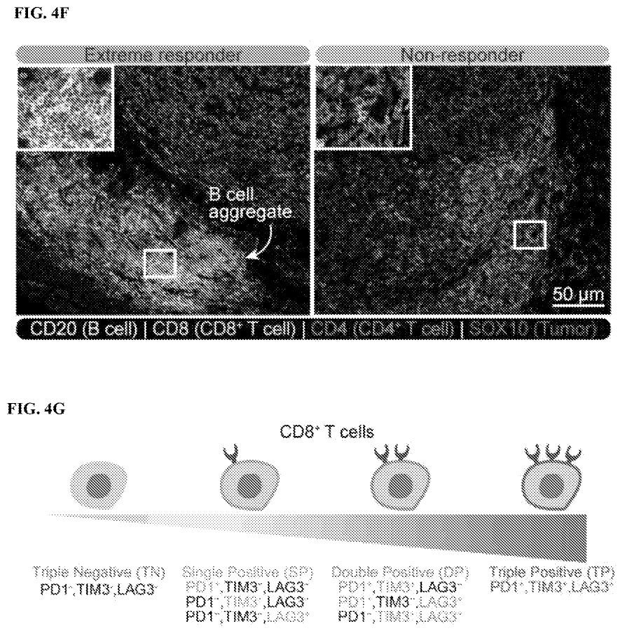

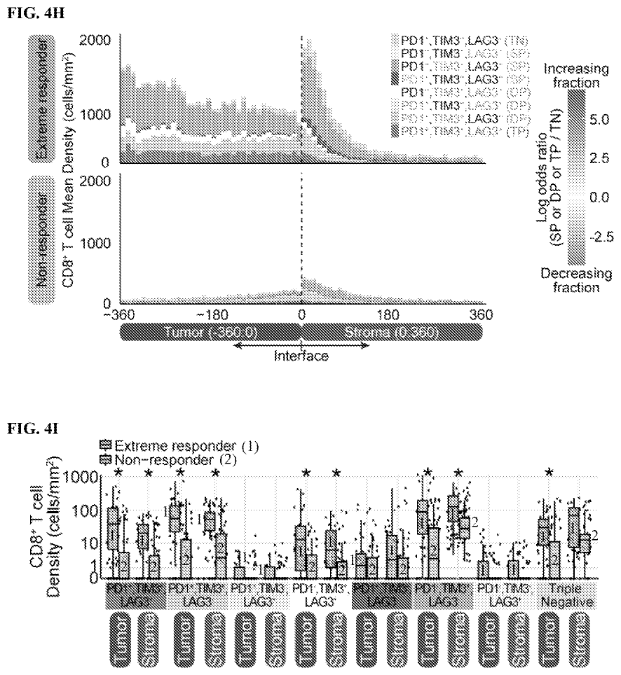

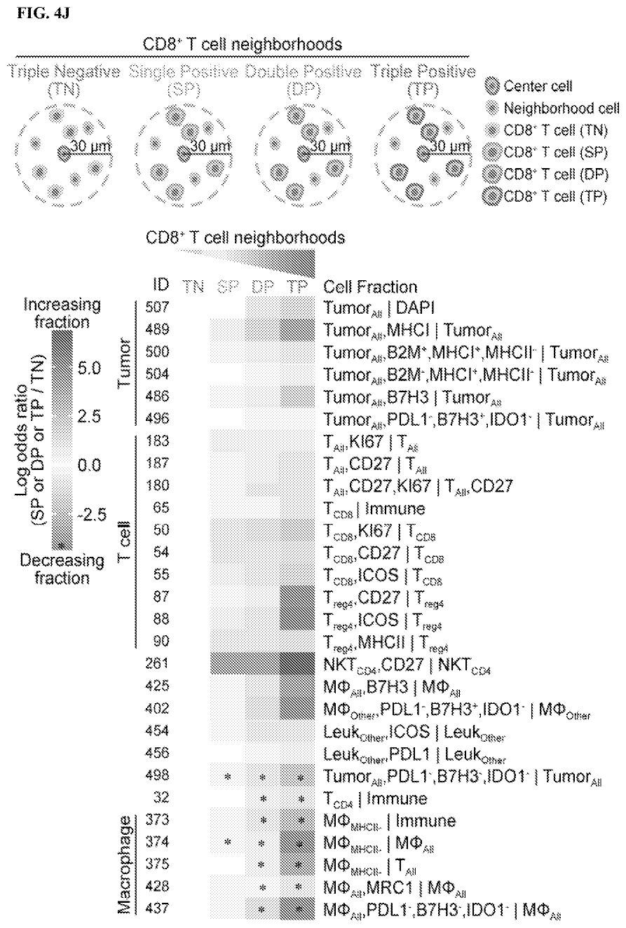

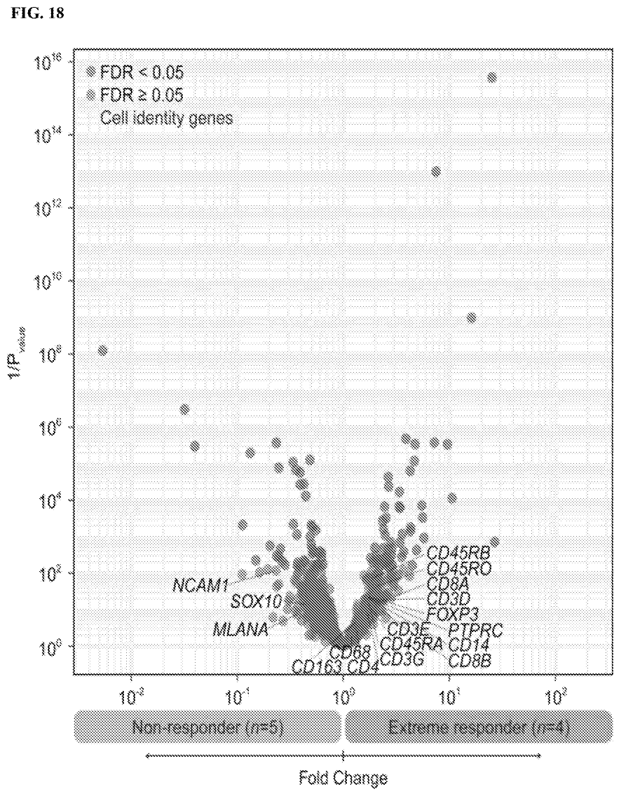

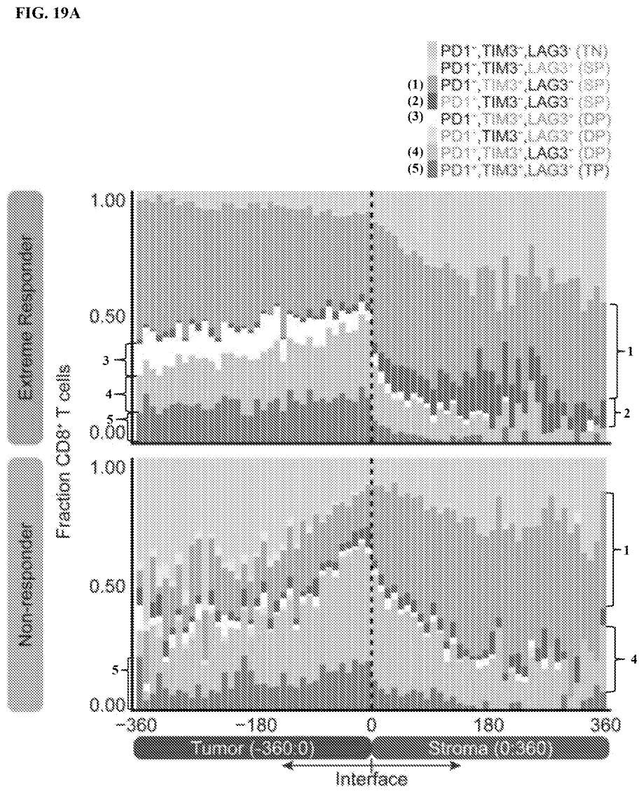

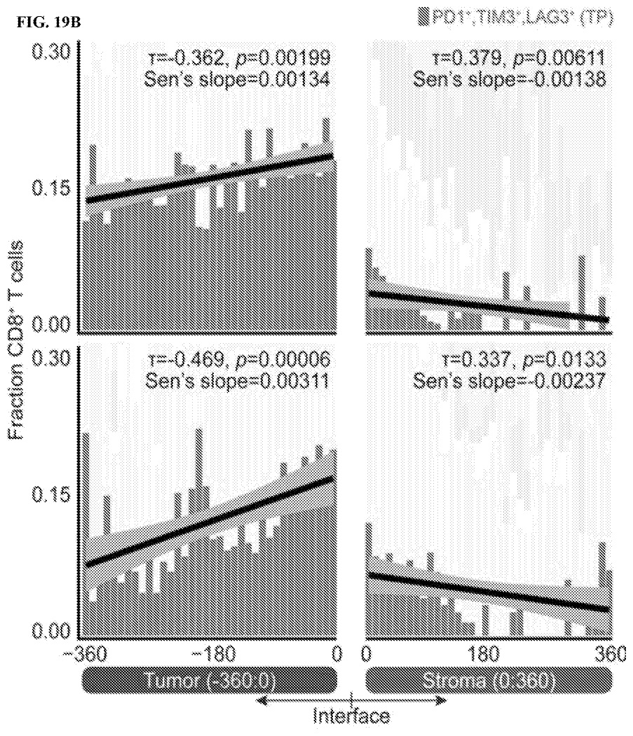

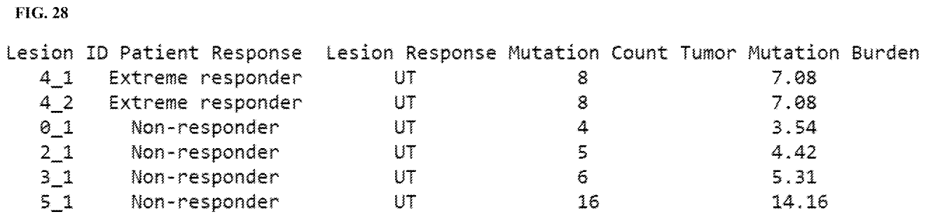

We next examined the expression of immune-related genes in untreated lesions from extreme responders and non-/mixed responders where we found differential expression of 96 genes. Untreated lesions from extreme responders showed uregulation of several genes associated with IFNγ and IFNα signaling, antigen presentation, IL-2 response, tertiary lymphoid structures (TLS), and T-cell dysfunction (FIG. 4A). Some of these gene-expression signatures (e.g., IFNγ signature and TLS) have been associated with clinical response to immune checkpoint blockade (ICB) in melanoma (15, 16). We did not observe significant expression changes in genes that identify immune cell populations, suggesting that upregulation of these gene-expression signatures in extreme responders was not simply a reflection of higher immune cell counts (FIG. 18). Given that tumor mutation burden (TMB) has been predictive of response to ICB (17), we determined TMB in our untreated lesions, but we found no instances of hypermutation (FIG. 28).

One of the TLS-related and upregulated genes in untreated lesions from extreme responders was CXCL13, a B-cell attractant that is secreted by dysfunctional CD8⁺ T cells (18, 19) and has been linked to clinical response to ICB (20). We therefore examined the spatial distribution of B cells and exhausted CD8⁺ T cells relative to the tumor-stroma interface (+/−360 μm) in untreated lesions (FIG. 4B). We had previously observed increased densities of B cells in untreated lesions from extreme responders (FIG. 14). Upon examining the spatial distribution of the B cells, we found that these B cells were predominately in the stroma of extreme responders following intralesional IL-2 (FIG. 4C). Although our review of H&E stains of the untreated lesions did not identify TLS, using multiplexed IF we observed a greater number of CD20⁺ B-cell aggregates, which were surrounded by both CD8⁺ and CD4⁺ T cells, in untreated lesions from extreme responders (FIGS. 4D-4F).

Next we examined the degree of CD8⁺ T-cell exhaustion (FIG. 4G) as a function of their spatial distribution relative to the tumor-stroma interface (FIG. 4H). In untreated lesions, we observed increasing fractions of PD-1⁺LAG-3⁺TIM-3⁺ exhausted CD8⁺ T cells, as CD8⁺ T cells approach the tumor interface from both the stroma (-360 μm) and from within the tumor (+360 μm, FIGS. 19A-19B). We observed increased densities of several CD8⁺ T-cell populations (i.e., expressing different combinations of PD-1, LAG-3, and TIM-3) in both the tumor (−360:0 μm) and stroma regions (0:360 μm) of extreme responders compared to non-/mixed responders (FIG. 4I).

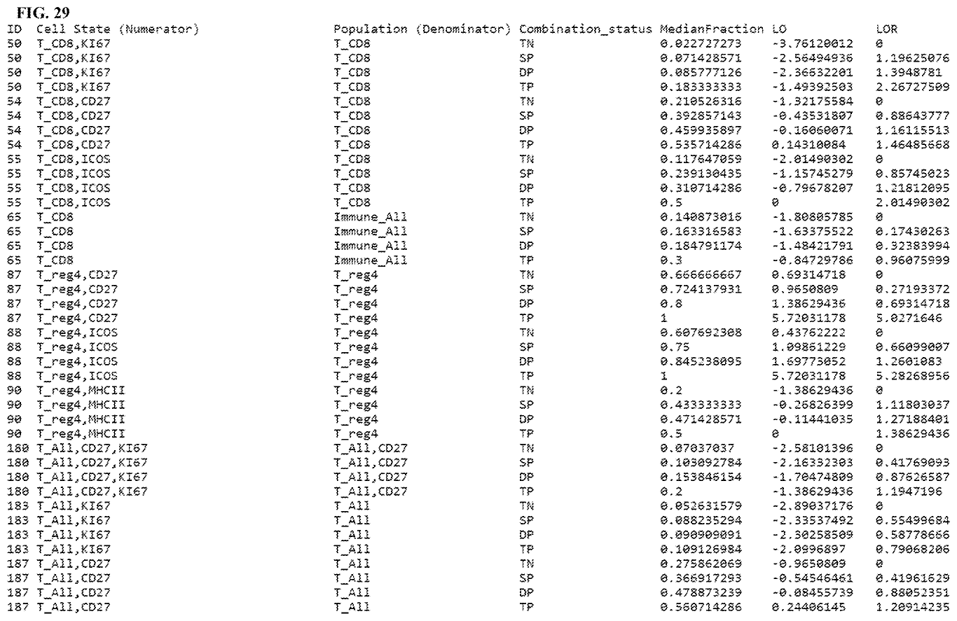

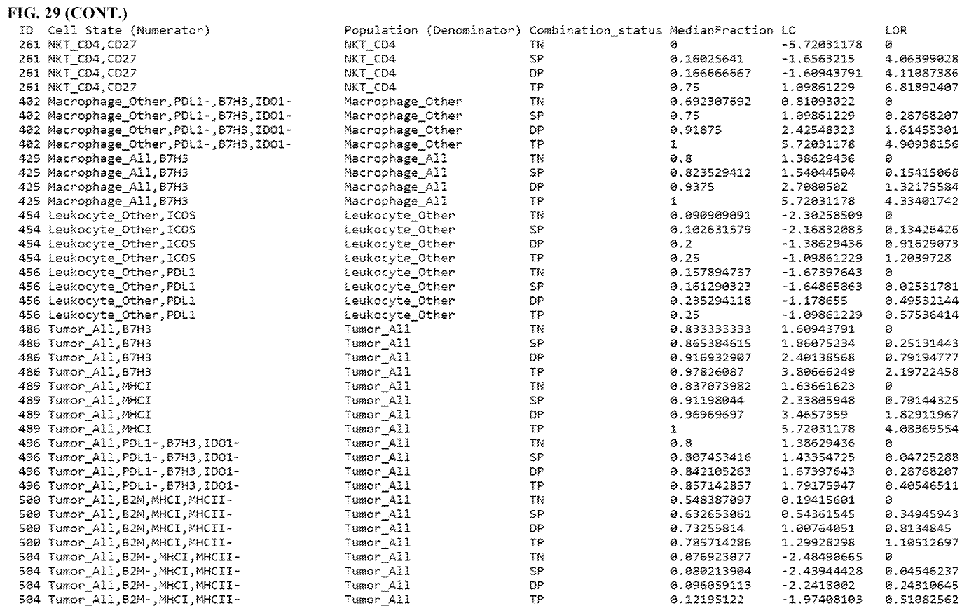

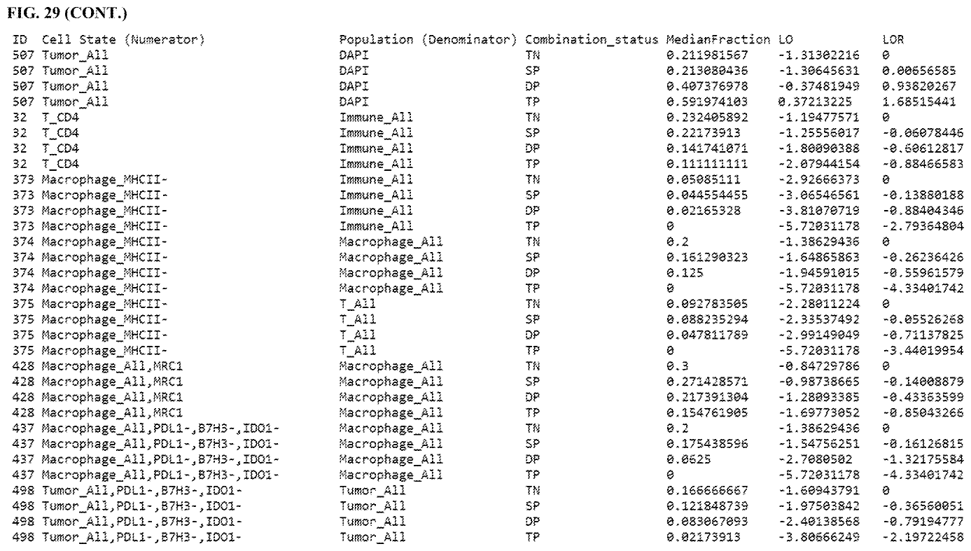

To identify potential contributors of CD8⁺ T-cell exhaustion, we also characterized the cellular neighborhood of CD8⁺ T cells as they progress from a triple-negative (PD-1⁻LAG-3⁻TIM-3⁻) to triple-positive (PD-1⁺LAG-3⁺TIM-3⁺) state (FIG. 4J, FIG. 29). As CD8⁺ T cells become exhausted (PD-1⁺LAG-3⁺TIM-3⁺), their neighborhood is characterized by increasing fractions of B2M⁺MHC-I⁺MHC-II⁻ tumor cells, proliferating/activated (Ki67⁺CD27⁺ICOS⁺) T and NK cells, B7-H3⁺ macrophages and tumor cells, and decreasing fractions of CD4⁺ T cells, MHC-II⁻ macrophages, and PD-L1⁻ B7-H3⁻IDO-1⁻ macrophage and tumor cells in their cellular neighborhoods.

In addition to widespread tumor-cell MHC-I expression, untreated lesions from “extreme responders” showed hallmarks of a tumor-reactive microenvironment, with stromal B-cell aggregates, increased expression of IFNγ, IFNα, and IL-2 response-related genes, and CD8⁺ T cells with an “exhausted” phenotype. The immune-cell infiltrate after complete eradication of all tumor cells, on the other hand, was characterized by CD8⁺ T cells with a “non-exhausted” phenotype. This study combined in situ single-cell profiling with bulk RNA and DNA profiling from adjacent unstained tissue sections of FFPE tumor, a biospecimen source that is widely available for the majority of cancer patients. This approach allowed identification of therapy-associated cell states and gene-expression signatures and generate immunotherapy response biomarker hypotheses.

These results demonstrate that the absence of membranous MHC-I in tumor cells is associated with the failure to respond to IL-2. This finding emerged from multi-dimensional analysis of in-transit metastases from 7 melanoma patients receiving intralesional IL-2 and was subsequently confirmed in an independent multi-institutional validation cohort of 19 patients with metastatic melanoma. Of note, melanoma patients in the validation cohort had received IL-2 therapy in either an intralesional or high-dose systemic formulation, broadening the impact of this biomarker to melanoma patients receiving systemic IL-2.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. F. Meier, S. Will, U. Ellwanger, B. Schlagenhauff, B. Schittek, G. Rassner, C. Garbe, Metastatic pathways and time courses in the orderly progression of cutaneous melanoma. Br. J. Dermatol. 147, 62-70 (2002).
2 D. G. Coit, J. A. Thompson, A. Algazi, R. Andtbacka, C. K. Bichakjian, W. E. Carson, G. A. Daniels, D. DiMaio, M. Ernstoff, R. C. Fields, M. D. Fleming, R. Gonzalez, V. Guild, A. C. Halpern, F. S. Hodi, R. W. Joseph, J. R. Lange, M. C. Martini, M. A. Materin, A. J. Olszanski, M. I. Ross, A. K. Salama, J. Skitzki, J. Sosman, S. M. Swetter, K. K. Tanabe, J. F. Torres-Roca, V. Trisal, M. M. Urist, N. McMillian, A. Engh, Melanoma, Version 2.2016, NCCN Clinical Practice Guidelines in Oncology. J. Natl. Compr. Cancer Netw. JNCCN. 14, 450-473 (2016).
3 B. A. Byers, C. F. Temple-Oberle, V. Hurdle, J. G. Mckinnon, Treatment of in-transit melanoma with intra-lesional interleukin-2: a systematic review. J. Surg. Oncol. 110, 770-775 (2014).
4. A. Mullard, Restoring IL-2 to its cancer immunotherapy glory. Nat. Rev. Drug Discov. 20, 163-165 (2021).
5. T. Read, M. Lonne, D. S. Sparks, M. David, M. Wagels, H. Schaider, H. P. Soyer, B. M. Smithers, A systematic review and meta-analysis of locoregional treatments for in-transit melanoma. J. Surg. Oncol. 119, 887-896 (2019).
6. P. Radny, U. M. Caroli, J. Bauer, T. Paul, C. Schlegel, T. K. Eigentler, B. Weide, M. Schwarz, C. Garbe, Phase II trial of intralesional therapy with interleukin-2 in soft-tissue melanoma metastases. Br. J. Cancer. 89, 1620-1626 (2003).
7. S. Hassan, T. M. Petrella, T. Zhang, S. Kamel-Reid, F. Nordio, A. Baccarelli, S. Sade, K. Naert, A. A. Habeeb, D. Ghazarian, F. C. Wright, Pathologic complete response to intralesional interleukin-2 therapy associated with improved survival in melanoma patients with in-transit disease. Ann. Surg. Oncol. 22, 1950-1958 (2015).
8 A. Zehir, R. Benayed, R. H. Shah, A. Syed, S. Middha, H. R. Kim, P. Srinivasan, J. Gao, D. Chakravarty, S. M. Devlin, M. D. Hellmann, D. A. Barron, A. M. Schram, M. Hameed, S. Dogan, D. S. Ross, J. F. Hechtman, D. F. DeLair, J. Yao, D. L. Mandelker, D. T. Cheng, R. Chandramohan, A. S. Mohanty, R. N. Ptashkin, G. Jayakumaran, M. Prasad, M. H. Syed, A. B. Rema, Z. Y. Liu, K. Nafa, L. Borsu, J. Sadowska, J. Casanova, R. Bacares, I. J. Kiecka, A. Razumova, J. B. Son, L. Stewart, T. Baldi, K. A. Mullaney, H. Al-Ahmadie, E. Vakiani, A. A. Abeshouse, A. V. Penson, P. Jonsson, N. Camacho, M. T. Chang, H. H. Won, B. E. Gross, R. Kundra, Z. J. Heins, H.-W. Chen, S. Phillips, H. Zhang, J. Wang, A. Ochoa, J. Wills, M. Eubank, S. B. Thomas, S. M. Gardos, D. N. Reales, J. Galle, R. Durany, R. Cambria, W. Abida, A. Cercek, D. R. Feldman, M. M. Gounder, A. A. Hakimi, J. J. Harding, G. Iyer, Y. Y. Janjigian, E. J. Jordan, C. M. Kelly, M. A. Lowery, L. G. T. Morris, A. M. Omuro, N. Raj, P. Razavi, A. N. Shoushtari, N. Shukla, T. E. Soumerai, A. M. Varghese, R. Yaeger, J. Coleman, B. Bochner, G. J. Riely, L. B. Saltz, H. I. Scher, P. J. Sabbatini, M. E. Robson, D. S. Klimstra, B. S. Taylor, J. Baselga, N. Schultz, D. M. Hyman, M. E. Arcila, D. B. Solit, M. Ladanyi, M. F. Berger, Mutational landscape of metastatic cancer revealed from prospective clinical sequencing of 10,000 patients. Nat. Med. 23, 703-713 (2017).
9. M. J. Gerdes, C. J. Sevinsky, A. Sood, S. Adak, M. O. Bello, A. Bordwell, A. Can, A. Corwin, S. Dinn, R. J. Filkins, D. Hollman, V. Kamath, S. Kaanumalle, K. Kenny, M. Larsen, M. Lazare, Q. Li, C. Lowes, C. C. McCulloch, E. McDonough, M. C. Montalto, Z. Pang, J. Rittscher, A. Santamaria-Pang, B. D. Sarachan, M. L. Seel, A. Seppo, K. Shaikh, Y. Sui, J. Zhang, F. Ginty, Highly multiplexed single-cell analysis of formalin-fixed, paraffin-embedded cancer tissue. Proc. Natl. Acad. Sci. 110, 11982-11987 (2013).
10. A. Sood, A. M. Miller, E. Brogi, Y. Sui, J. Armenia, E. McDonough, A. Santamaria-Pang, S. Carlin, A. Stamper, C. Campos, Z. Pang, Q. Li, E. Port, T. G. Graeber, N. Schultz, F. Ginty, S. M. Larson, I. K. Mellinghoff, Multiplexed immunofluorescence delineates proteomic cancer cell states associated with metabolism. JCI Insight. 1, 87030 (2016).
11. N. Acharya, C. Sabatos-Peyton, A. C. Anderson, Tim-3 finds its place in the cancer immunotherapy landscape. J. Immunother. Cancer. 8, e000911 (2020).
12. Y. Wolf, A. C. Anderson, V. K. Kuchroo, TIM3 comes of age as an inhibitory receptor. Nat. Rev. Immunol. 20, 173-185 (2020).
13. G. Cesana, G. Iodice, S. Cohen, D. Moroziewicz, J. Mitcham, J. Stoutenburg, K. Cheung, C. Hesdorffer, S. Kim-Schulze, H. Kaufman, Characterization of CD4+CD25+ Regulatory T Cells in Patients Treated With High-Dose Interleukin-2 for Metastatic Melanoma or Renal Cell Carcinoma. J. Clin. Oncol. Off. J. Am. Soc. Clin. Oncol. 24, 1169-77 (2006).
14. D. H. Raulet, S. Gasser, B. G. Gowen, W. Deng, H. Jung, Regulation of Ligands for the NKG2D Activating Receptor. Annu. Rev. Immunol. 31, 413-441 (2013).
15. C. S. Grasso, J. Tsoi, M. Onyshchenko, G. Abril-Rodriguez, P. Ross-Macdonald, M. Wind-Rotolo, A. Champhekar, E. Medina, D. Y. Torrejon, D. S. Shin, P. Tran, Y. J. Kim, C. Puig-Saus, K. Campbell, A. Vega-Crespo, M. Quist, C. Martignier, J. J. Luke, J. D. Wolchok, D. B. Johnson, B. Chmielowski, F. S. Hodi, S. Bhatia, W. Sharfman, W. J. Urba, C. L. Slingluff, A. Diab, J. B. A. G. Haanen, S. M. Algarra, D. M. Pardoll, V. Anagnostou, S. L. Topalian, V. E. Velculescu, D. E. Speiser, A. Kalbasi, A. Ribas, Conserved Interferon-y Signaling Drives Clinical Response to Immune Checkpoint Blockade Therapy in Melanoma. Cancer Cell. 38, 500-515.e3 (2020).
16. R. Cabrita, M. Lauss, A. Sanna, M. Donia, M. Skaarup Larsen, S. Mitra, I. Johansson, B. Phung, K. Harbst, J. Vallon-Christersson, A. van Schoiack, K. Lövgren, S. Warren, K. Jirström, H. Olsson, K. Pietras, C. Ingvar, K. Isaksson, D. Schadendorf, H. Schmidt, L. Bastholt, A. Carneiro, J. A. Wargo, I. M. Svane, G. Jönsson, Tertiary lymphoid structures improve immunotherapy and survival in melanoma. Nature. 577, 561-565 (2020).
17. M. Yarchoan, L. A. Albacker, A. C. Hopkins, M. Montesion, K. Murugesan, T. T. Vithayathil, N. Zaidi, N. S. Azad, D. A. Laheru, G. M. Frampton, E. M. Jaffee, PD-L1 expression and tumor mutational burden are independent biomarkers in most cancers. JCI Insight. 4 (2019), doi: 10.1172/jci.insight. 126908.
18. A. M. van der Leun, D. S. Thommen, T. N. Schumacher, CD8⁺ T cell states in human cancer: insights from single-cell analysis. Nat. Rev. Cancer. 20, 218-232 (2020).
19. H. Li, A. M. van der Leun, I. Yofe, Y. Lubling, D. Gelbard-Solodkin, A. C. J. van Akkooi, M. van den Braber, E. A. Rozeman, J. B. A. G. Haanen, C. U. Blank, H. M. Horlings, E. David, Y. Baran, A. Bercovich, A. Lifshitz, T. N. Schumacher, A. Tanay, I. Amit, Dysfunctional CD8 T Cells Form a Proliferative, Dynamically Regulated Compartment within Human Melanoma. Cell. 176, 775-789.e18 (2019).
20. K. Litchfield, J. L. Reading, C. Puttick, K. Thakkar, C. Abbosh, R. Bentham, T. B. K. Watkins, R. Rosenthal, D. Biswas, A. Rowan, E. Lim, M. A. Bakir, V. Turati, J. A. Guerra-Assunção, L. Conde, A. J. S. Furness, S. K. Saini, S. R. Hadrup, J. Herrero, S.-H. Lee, P. V. Loo, T. Enver, J. Larkin, M. D. Hellmann, S. Turajlic, S. A. Quezada, N. McGranahan, C. Swanton, Meta-analysis of tumor- and T cell-intrinsic mechanisms of sensitization to checkpoint inhibition. Cell. 184, 596-614.e14 (2021).
21. D. Chowell, L. G. T. Morris, C. M. Grigg, J. K. Weber, R. M. Samstein, V. Makarov, F. Kuo, S. M. Kendall, D. Requena, N. Riaz, B. Greenbaum, J. Carroll, E. Garon, D. M. Hyman, A. Zehir, D. Solit, M. Berger, R. Zhou, N. A. Rizvi, T. A. Chan, Patient HLA class I genotype influences cancer response to checkpoint blockade immunotherapy. Science. 359, 582-587 (2018).
22 B. C. Miller, D. R. Sen, R. Al Abosy, K. Bi, Y. V. Virkud, M. W. LaFleur, K. B. Yates, A. Lako, K. Felt, G. S. Naik, M. Manos, E. Gjini, J. R. Kuchroo, J. J. Ishizuka, J. L. Collier, G. K. Griffin, S. Maleri, D. E. Comstock, S. A. Weiss, F. D. Brown, A. Panda, M. D. Zimmer, R. T. Manguso, F. S. Hodi, S. J. Rodig, A. H. Sharpe, W. N. Haining, Subsets of exhausted CD8+ T cells differentially mediate tumor control and respond to checkpoint blockade. Nat. Immunol. 20, 326-336 (2019).
23. S. J. Rodig, D. Gusenleitner, D. G. Jackson, E. Gjini, A. Giobbie-Hurder, C. Jin, H. Chang, S. B. Lovitch, C. Horak, J. S. Weber, J. L. Weirather, J. D. Wolchok, M. A. Postow, A. C. Pavlick, J. Chesney, F. S. Hodi, MHC proteins confer differential sensitivity to CTLA-4 and PD-1 blockade in untreated metastatic melanoma. Sci. Transl. Med. 10 (2018), doi: 10.1126/scitranslmed.aar3342.
24. R. Spolski, P. Li, W. J. Leonard, Biology and regulation of IL-2: from molecular mechanisms to human therapy. Nat. Rev. Immunol. 18, 648-659 (2018).
25. O. Boyman, J. Sprent, The role of interleukin-2 during homeostasis and activation of the immune system. Nat. Rev. Immunol. 12, 180-190 (2012).
26. V. Kalia, S. Sarkar, Regulation of Effector and Memory CD8⁺ T Cell Differentiation by IL-2-A Balancing Act. Front. Immunol. 9, 2987 (2018).

1. A method for selecting a melanoma cancer patient for treatment with IL-2 therapy comprising
a. detecting the presence of wild-type polynucleotides in at least one MHC class I gene in a biological sample obtained from the melanoma cancer patient; and
b. administering to the melanoma cancer patient an effective amount of a therapeutic IL-2 composition.

2. The method of claim 1, wherein the at least one MHC class I gene is selected from the group consisting of HLA-A, HLA-B, HLA-C, and B2M.

3. The method of claim 1, wherein the wild-type polynucleotides are detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH).

4. The method of claim 1, wherein the biological sample comprises genomic DNA, cDNA, RNA, and/or mRNA.

5. A method for selecting a melanoma cancer patient for treatment with IL-2 therapy comprising
a. detecting mRNA and/or polypeptide expression levels of at least one MHC class I gene in a biological sample obtained from the melanoma cancer patient that are comparable to a control sample obtained from a healthy subject or a predetermined threshold; and
b. administering to the melanoma cancer patient an effective amount of a therapeutic IL-2 composition.

6. A method for treating melanoma in a patient in need thereof comprising
administering to the patient an effective amount of a therapeutic IL-2 composition,
wherein mRNA and/or polypeptide expression levels of one or more of HLA-A, HLA-B, HLA-C, and B2M in a biological sample obtained from the patient are comparable to a control sample obtained from a healthy subject or a predetermined threshold.

7. The method of claim 5, wherein mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH).

8. The method of claim 6, wherein polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

9. The method of claim 6, wherein the biological sample comprises skin tissue, soft tissue, lymph nodes, whole blood, plasma, or serum.

10. The method of claim 6, wherein the biological sample comprises tumor cells and/or macrophages.

11. The method of claim 10, wherein the tumor cells exhibit membranous expression of MHC class I genes.

12. The method of claim 6, wherein the patient exhibits stage I, stage II, stage III or stage IV melanoma.

13. The method of claim 13, wherein the melanoma is metastatic or localized.

14. The method of claim 6, wherein the therapeutic IL-2 composition comprises wild-type human IL-2, a variant human IL-2 or a chimeric IL-2 fusion polypeptide.

15. The method of claim 6, wherein the wild-type human IL-2 or the chimeric IL-2 fusion polypeptide comprises the amino acid sequence of SEQ ID NO: 1 or SEQ ID NO: 2.

16. The method of claim 14, wherein the chimeric IL-2 fusion polypeptide comprises a Fc domain, a serum albumin domain, a polyethylene glycol (PEG) domain, transferrin, a serum albumin binding protein, a serum immunoglobulin binding protein and a fibronectin (Fn)-based scaffold domain.

17. The method of claim 6, wherein the therapeutic IL-2 composition is administered systemically, intratumorally or intravenously.

18. The method of claim 6, wherein the patient has a high or low tumor mutation burden (TMB).

19. The method of claim 6, wherein the melanoma is superficial spreading, nodular, lentigo maligna, acral lentiginous, amelanotic, or desmoplastic.

20. The method of claim 6, wherein the effective amount of the therapeutic IL-2 composition is administered as a series of 2-12 injections.
